(19) United States
(12) Patent Application Publication
(10) Pub. No.: US 2026/0097015 A1
(43) Pub. Date: Apr. 9, 2026

(54) EXTENDED RELEASE FORMULATIONS COMPRISING SUBSTITUTED INDAZOLE PROPIONIC ACID DERIVATIVE COMPOUNDS AND USES THEREOF

(71) Applicant: Pfizer Inc., New York, NY (US)

(72) Inventors: Alfred Berchielli, East Lyme, CT (US); Brett John Kopina, Waterford, CT (US)

(21) Appl. No.: 19/348,897

(22) Filed: Oct. 3, 2025

Related U.S. Application Data
(60) Provisional application No. 63/703,265, filed on Oct. 4, 2024.

(51) Int. Cl. A61K 31/416; A61K 9/00; A61K 9/50; A61K 31/427; A61K 31/437; A61K 31/4439; A61K 31/7056

(52) U.S. Cl. CPC A61K 31/416; A61K 9/0053; A61K 9/5015; A61K 9/5026; A61K 31/427; A61K 31/437; A61K 31/4439; A61K 31/7056

(57) ABSTRACT

The invention relates to extended-release formulations comprising substituted indazole propionic acid derivatives, pharmaceutically acceptable salts, tautomers, or pharmaceutically acceptable salts of the tautomers thereof that can activate adenosine 5′-monophosphate-activated protein kinase (AMPK). The invention further relates to methods of treating a condition comprising administering an extended-release formulation comprising AMPK-activating substituted indazole propionic acid derivatives, pharmaceutically acceptable salts, tautomers, or pharmaceutically acceptable salts of the tautomers thereof.

### BACKGROUND OF THE INVENTION

Adenosine 5′-monophosphate-activated protein kinase (AMPK) is a highly conserved serine/threonine kinase that functions as a central regulator of energy homeostasis. AMPK has been demonstrated to mediate multiple pathways within intestinal epithelial cells, including direct modulation of substrates involved in tightjunction stability, polarity, differentiation, nutrient transport, and autophagy. Strengthening the intestinal barrier could have therapeutic potential for metabolic- and inflammatory-related diseases associated with intestinal permeability or a “leaky gut”. Given the functional attributes of AMPK in energy and tissue homeostasis, there is a need for potent and direct, gut-targeted activators of AMPK to treat conditions associated with AMPK activation.

### SUMMARY OF THE INVENTION

Disclosed herein is a composition comprising: a) a compound of Formula (I):

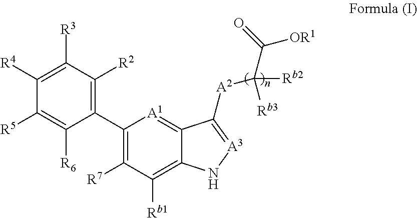

a pharmaceutically acceptable salt, a tautomer, or a pharmaceutically acceptable salt of the tautomer thereof, wherein:
   A¹ is CR⁸, or N;
   A² is CH₂, CHD, CD₂, S, O, or NH;
   A³ is CH, CD, or N;
   R¹ is H, D, C₁₋₈alkyl, C₃₋₆cycloalkyl, or 4-6 membered heterocycloalkyl, each of which is optionally substituted;
   R², R³, R⁵, and R⁶ are each independently H, D, OH, or halogen;
   R⁴ is monocyclic aryl, bicyclic aryl, monocyclic heteroaryl, or bicyclic heteroaryl, each of which is optionally substituted with R⁹, R¹⁰, R¹¹, R¹², or R¹³,
      wherein R⁹, R¹⁰, R¹¹, R¹², and R¹³ are each independently H, D, halogen, CN, oxo, C₁₋₈alkyl, C₃₋₆cycloalkyl, C₀₋₆alkylene-OR^{x}, C₁₋₆haloalkylene-OR^{x}, C₀₋₆alkylene(C₀₋₆haloalkyl)NR^{x}R^{y}, C₁₋₆alkylene(C₁₋₆haloalkyl)NR^{x}R^{y}, 4-6 membered heterocycloalkyl, C(O)OR^{x}, C₀₋₆alkylene-C(O)NRR^{y}, OC₁₋₃alkylene-heterocycloalkyl, OC₁₋₃alkylene-C(O)NR^{x}R^{y}, O(C₁₋₆alkyl)SO₂NR^{x}NR^{y}, NR^{x}R^{y}, NHSO₂R^{x}, SR^{x}, S—C₁₋₆alkylene-C(O)NR^{x}R^{y}, S(O)R^{x}R^{y}, SO₂R^{x}, SO₂NR^{x}R^{y}, S(O)(NR^{x})R^{y}, S(O)(NR^{x})R^{y}, or SO₂R^{x};
   wherein each R^{x} and R^{y} is independently H, D, C₁₋₆alkyl, C₁₋₆haloalkyl, C₁₋₆alkoxy, C₃₋₆cycloalkyl, C₁₋₆alkylene-amide, OC₀₋₂alkylene-heterocycloalkyl, 4-6 membered heterocycloalkyl, C(O)C₁₋₆alkyl, imino, or C₁₋₆alkylsulfonyl; or R^{x} and R^{y} together with the atoms to which R^{x} and R^{y} are bound can form an optionally substituted ring;
   R⁷ is C₁₋₃alkyl, C₃₋₆cycloalkyl, cyano, or halogen;
   R^{b1}, R^{b2}, and R^{b3} are each independently H or D;
   R⁸ is H, D, or halogen; and
   n is 0, 1, or 2;
b) a matrix component; and c) a release modifier, wherein the composition is an extended-release composition.

Also disclosed herein is a method for treating a condition, comprising administering to a subject in need thereof a therapeutically effective amount of the composition of the disclosure, wherein the condition is an inflammatory condition, an autoimmune condition, or a functional gastrointestinal disorder. Further disclosed herein is use of a composition disclosed herein in the manufacture of a medicament for the treatment of an inflammatory condition, an autoimmune condition, or a functional gastrointestinal disorder.

Disclosed herein is a method of making a composition of claim 1, comprising: a) admixing, blending, and milling the compound of Formula (I), a pharmaceutically acceptable salt, a tautomer, or a pharmaceutically acceptable salt of the tautomer thereof; the matrix component; and the release modifier to form a mixture; and b) subjecting the mixture to a melt-spray-congeal process to form microsphere particles.

Further disclosed herein is a compound of Formula (I):

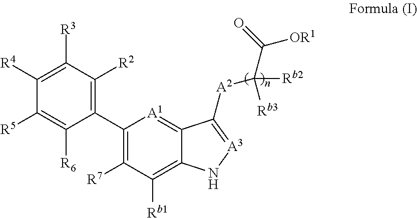

or a pharmaceutically acceptable salt, a tautomer, or a pharmaceutically acceptable salt of the tautomer thereof, wherein:
   A¹ is CR⁸, or N;
   A² is CH₂, CHD, CD₂, S, O, or NH;
   A³ is CH, CD, or N;
   R¹ is H, D, C₁₋₈alkyl, C₃₋₆cycloalkyl, or 4-6 membered heterocycloalkyl, each of which is optionally substituted;
   R², R³, R⁵, and R⁶ are each independently H, D, OH, or halogen;
   R⁴ is monocyclic aryl, bicyclic aryl, monocyclic heteroaryl, or bicyclic heteroaryl, each of which is optionally substituted with R⁹, R¹⁰, R¹¹, R¹², or R¹³,
      wherein R⁹, R¹⁰, R¹¹, R¹², and R¹³ are each independently H, D, halogen, CN, oxo, C₁₋₈alkyl, C₃₋₆cycloalkyl, C₀₋₆alkylene-OR^{x}, C₁₋₆haloalkylene-OR^{x}, C₀₋₆alkylene(C₀₋₆haloalkyl)NR^{x}R^{y}, C₁₋₆alkylene(C₁₋₆haloalkyl)NRR^{y}, 4-6 membered heterocycloalkyl, C(O)OR^{x}, C₀₋₆alkylene-C(O)NRR^{y}, OC₁₋₃alkylene-heterocycloalkyl, OC₁₋₃alkylene-C(O)NR^{x}R^{y}, O(C₁₋₆alkyl)SO₂NR^{x}NR^{y}, NR^{x}R^{y}, NHSO₂R^{x}, SR^{x}, S—C₁₋₆alkylene-C(O)NR^{x}R^{y}, S(O)R^{x}R^{y}, SO₂R^{x}, SO₂NR^{x}R^{y}, S(O)(NR^{x})R^{y}, S(O)(NR^{x})R^{y}, or SO₂R^{x};
   wherein each R^{x} and R^{y} is independently H, D, C₁₋₆alkyl, C₁₋₆haloalkyl, C₁₋₆alkoxy, C₃₋₆cycloalkyl, C₁₋₆alkylene-amide, OC₀₋₂alkylene-heterocycloalkyl, 4-6 membered heterocycloalkyl, C(O)C₁₋₆alkyl, imino, or C₁₋₆alkylsulfonyl; or R^{x} and R^{y} together with the atoms to which R^{x} and R^{y} are bound can form an optionally substituted ring;
   R⁷ is C₁₋₃alkyl, C₃₋₆cycloalkyl, cyano, or halogen;
   R^{b1}, R^{b2}, and R^{b3} are each independently H or D;
   R³ is H, D, or halogen; and
   n is 0, 1, or 2,
      wherein the compound is amorphous.

It is to be understood that both the foregoing general description and the following detailed description are exemplary and explanatory only and are not restrictive of the invention, as claimed.

### BRIEF DESCRIPTION OF THE DRAWINGS

FIG. 1 illustrates a scheme of a general spray congealing apparatus for the production of microparticles

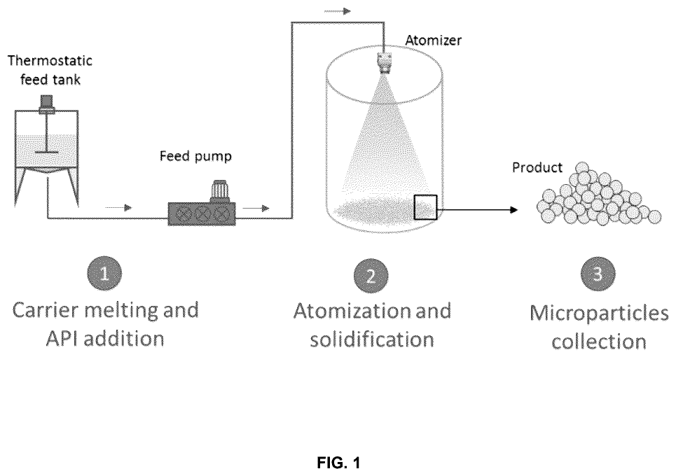

FIG. 2 illustrates differences in the time vs change in dissolution rate of a formulation comprising: 1) 10:9:81 Compound 1/poloxamer 407/stearyl alcohol; 2) 10:9:81 Compound 1/crospovidone/stearyl alcohol; 3) 10:9:81 Compound 1/crospovidone/polyglyceryl-4-stearate; and 4) 10:9:81 Compound 1/sodium starch glycolate/stearyl alcohol.

FIG. 3 shows the crospovidone (% w/w) in inactive ingredients vs time to 80% release in fasted state simulated intestinal fluid for a formulation comprising crospovidone with a surface area of 1.4 m²/g or crospovidone with a surface area of >6 m²/g.

FIG. 4 shows Compound 1 loading in formulation (% w/w) vs in vitro dissolution time to 80% release in fasted state simulated intestinal fluid.

FIG. 5 depicts a PXRD diffractogram of a compound of the disclosure.

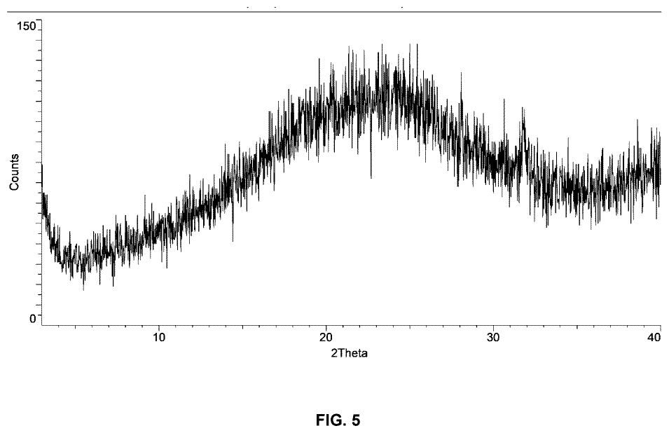

FIG. 6 shows a modulated differential scanning calorimetry measurement of a compound of the disclosure.

### DETAILED DESCRIPTION OF THE INVENTION

The present invention may be understood more readily by reference to the following detailed description of the embodiments of the invention and the Examples included herein. It is to be understood that this invention is not limited to specific synthetic methods of making that may of course vary. It is to be also understood that the terminology used herein is for the purpose of describing specific embodiments only and is not intended to be limiting.

Adenosine 5′-monophosphate-activated protein kinase (AMPK) is a highly conserved serine/threonine kinase that functions as a central regulator of energy homeostasis. AMPK exists as a heterotrimeric protein complex consisting of a catalytic α-subunit, scaffolding β-subunit, and regulatory γ-subunit. Multiple isoforms (e.g., two α, two β, three γ) encoded by different genes enable up to twelve possible AMPK heterotrimeric complexes, with each AMPK heterotrimeric complex having a distinct cellular and tissue expression profile. AMPK is activated by upstream kinases, including liver kinase β1 (LKB1) and calcium/calmodulin-dependent protein kinase β (CamKKp), which phosphorylate the Thr172 active site residue within the α-subunit of AMPK. AMPK is also activated when the ratio of intracellular adenosine monophosphate (AMP):adenosine triphosphate (ATP) or to a lesser extent adenosine diphosphate (ADP):ATP is increased under conditions of energetic stress, such as nutrient starvation, inflammation, and hypoxia. Upon activation, AMPK phosphorylates direct substrates involved in pathways that promote ATP production (e.g., fatty acid oxidation, glycolysis, glucose uptake, autophagy, and mitophagy) and inhibit ATP consumption (e.g., synthesis of glucose, lipids, and proteins; cell growth) to restore energy balance. Moreover, AMPK can regulate and reprogram metabolism through transcriptional changes by phosphorylating factors that induce or repress gene transcription.

Multiple approaches for pharmacologically activating AMPK exist, both direct and indirect. 5-aminoimidazole-4-carboxamide-1-β-D-ribofuranoside (AICAR) and metformin increase cytosolic AMP. AICAR functions as an AMP mimetic, whereas metformin indirectly increases cytosolic AMP by inhibiting mitochondrial respiration and the release of ATP. AMP can bind the γ-subunit of AMPK to allosterically activate AMPK. In contrast, direct AMPK agonists can bind the allosteric drug and metabolism (ADaM) site between the α and β subunits of AMPK to activate and protect AMPK from dephosphorylation. Both pan-β and β1-selective AMPK agonists have been described.

AMPK activity can be altered due to pathological conditions, including metabolic and inflammatory diseases, such as obesity, diabetes, cardiovascular disease, and cancer. Additionally, there is evidence that AMPK can promote and maintain intestinal barrier function. AMPK has been demonstrated to mediate multiple pathways within intestinal epithelial cells, including direct modulation of substrates involved in tight junction stability, polarity, differentiation, nutrient transport, and autophagy. Strengthening the intestinal barrier could have therapeutic potential for metabolic- and inflammatory-related diseases associated with intestinal permeability or a “leaky gut”. AMPK activators have been developed for systemic administration. Given the functional attributes of AMPK in energy and tissue homeostasis, there is a need for potent and direct, gut-targeted activators of AMPK to treat conditions associated with AMPK activation.

Disclosed herein are extended release formulations comprising AMPK-activating compounds, pharmaceutically acceptable salts, tautomers, or pharmaceutically acceptable salts of the tautomers thereof. Further disclosed herein are methods of preparing extended release formulations comprising AMPK-activating compounds or pharmaceutically acceptable salts, tautomers, or pharmaceutically acceptable salts of the tautomers thereof, and methods of administering the AMPK-activating compounds, a pharmaceutically acceptable salt thereof, a tautomer thereof, or a pharmaceutically acceptable salt of the tautomer thereof, in a subject in need thereof to treat a condition. In some embodiments, the extended release formulations disclosed herein may be used to treat a metabolic disorder, an inflammatory disorder, an autoimmune disorder, a disorder of gastrointestinal barrier dysfunction, a functional gastrointestinal disorder, a central nervous system disorder, an eating disorder, a nutritional disorder, or an allergy. In a preferred embodiment, the extended release formulations disclosed herein may be used to treat a metabolic disorder, an inflammatory disorder, an autoimmune disorder, a disorder of gastrointestinal barrier dysfunction, or a functional gastrointestinal disorder.

### Definitions

Unless otherwise defined herein, scientific and technical terms used in connection with the present invention have the meanings that are commonly understood by those of ordinary skill in the art. The invention described herein suitably may be practiced in the absence of any element(s) not specifically disclosed herein.

“Compounds of the invention” or “compounds of the disclosure” include compounds of Formula I, Ia, II, III, IVa-c, and V, and the novel intermediates used in the preparation thereof. One of ordinary skill in the art will appreciate that compounds of the invention include conformational isomers (e.g., cis and trans isomers) and all optical isomers (e.g., enantiomers and diastereomers), racemic, diastereomeric and other mixtures of such isomers, tautomers thereof, where they may exist. One of ordinary skill in the art will also appreciate that compounds of the invention include solvates, hydrates, isomorphs, polymorphs, esters, salt forms, prodrugs, and isotopically labelled versions thereof, where they may be formed.

As used herein, the singular form “a”, “an”, and “the” include plural references unless indicated otherwise. For example, “a” substituent includes one or more substituents.

As used herein, the term “about” when used to modify a numerically defined parameter (e.g., the dose of an AMPK-activating compound, a pharmaceutically acceptable salt, a tautomer, or a pharmaceutically acceptable salt of the tautomer thereof) means that the parameter may vary by as much as 10% below or above the stated numerical value for that parameter. For example, a dose of about 5 mg means 5 mg ±10%, i.e., it may vary between 4.5 mg and 5.5 mg.

The term “and/or” means one or more. For example, “X and/or Y” shall be understood to mean either “X and Y” or “X or Y” and shall be taken to provide explicit support for both meanings or for either meaning. Similarly, when more than 2 expressions are listed, such as in “X, Y and/or Z”, it shall be understood to mean either i) “X and Y”, “X, Y and Z”, “X and Z”, or “Y and Z”, or ii) “X or Y or Z” and shall be taken to provide explicit support for all meanings.

Any open valency appearing on a carbon, oxygen, sulfur, or nitrogen atom in the structures disclosed herein indicates the presence of a hydrogen, unless indicated otherwise.

If substituents are described as being “independently selected” from a group, each substituent is selected independent of the other. Each substituent therefore may be identical to or different from the other substituent(s).

“Optional” or “optionally” means that the subsequently described event or circumstance may, but need not occur, and the description includes instances where the event or circumstance occurs and instances in which it does not.

The terms “optionally substituted” and “substituted or unsubstituted” are used interchangeably to indicate that the particular group being described may have no non-hydrogen substituents (i.e., unsubstituted), or the group may have one or more non-hydrogen substituents (i.e., substituted). If not otherwise specified, the total number of substituents that may be present is equal to the number of H atoms present on the unsubstituted form of the group being described. Where an optional substituent is attached via a double bond, such as an oxo (=O) substituent, the group occupies two available valences, so the total number of other substituents that are included is reduced by two. In the case where optional substituents are selected independently from a list of alternatives, the selected groups may be the same or different. Throughout the disclosure, it will be understood that the number and nature of optional substituent groups will be limited to the extent that such substitutions make chemical sense to one of ordinary skill in the art.

“Halogen” or “halo” refers to fluoro, chloro, bromo and iodo (F, Cl, Br, I). In a preferred embodiment, “halo” refers to fluoro. In a preferred embodiment, “halo” refers to chloro.

“Cyano” refers to a substituent having a carbon atom joined to a nitrogen atom by a triple bond, i.e., —C≡N.

“Hydroxy” refers to an —OH group.

“Oxo” refers to a double bonded oxygen (═O).

The term C₁-Cₓ includes C₁-C₂, C₁-C₃ . . . C₁-Cₓ. By way of example only, a group designated as “C₁-C₄” indicates that there are one to four carbon atoms in the moiety, i.e., groups containing 1 carbon atom, 2 carbon atoms, 3 carbon atoms, or 4 carbon atoms. For example, “C₁-C₄ alkyl” indicates that there are one to four atom carbons in the alkyl group, i.e., the alkyl group is selected from methyl, ethyl, propyl, isopropyl, n-butyl, isobutyl, sec-butyl, and t-butyl.

The terms “carbocyclic” or “carbocycle” refer to a ring or ring system where the atoms forming the backbone of the ring are all carbon atoms. The term is distinguished from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. For example. Carbocycle includes cycloalkyl and aryl.

“Alkyl” refers to a saturated, monovalent aliphatic hydrocarbon radical that has a specified number of carbon atoms, including straight chain or branched chain groups. Alkyl groups may contain, but are not limited to, 1 to 12 carbon atoms (“C₁-C₁₂ alkyl”), 1 to 8 carbon atoms (“C₁-C₅ alkyl”), 1 to 6 carbon atoms (“C₁-C₆ alkyl”), 1 to 5 carbon atoms (“C₁-C₈alkyl”), 1 to 4 carbon atoms (“C₁-C₄ alkyl”), 1 to 3 carbon atoms (“C₁-C₃ alkyl”), or 1 to 2 carbon atoms (“C₁-C₂ alkyl”). Examples include, but are not limited to, methyl, ethyl, n-propyl, isopropyl, n-butyl, sec-butyl, isobutyl, tert-butyl, n-pentyl, isopentyl, neopentyl, n-hexyl, n-heptyl, n-octyl, and the like. Alkyl groups may be optionally substituted, unsubstituted, or substituted, as further defined herein.

The term “haloalkyl” refers to an alkyl group wherein at least one of the hydrogen atoms of the alkyl group has been replaced by at least one of the same or different halogen atoms. For example, “fluoroalkyl” means an alkyl as defined herein substituted with one, two or three fluoro atoms. Exemplary (C₁)fluoroalkyl compounds include fluoromethyl, difluoromethyl and trifluoromethyl; exemplary (C₂)fluoroalkyl compounds include 1-fluoroethyl, 2-fluoroethyl, 1,1-difluoroethyl, 1,2-difluoroethyl, 1,1,1-trifluoroethyl, 1,1,2-trifluoroethyl, and the like. Examples of fully substituted fluoroalkyl groups (also referred to as perfluoroalkyl groups) include trifluoromethyl (—CF₃) and pentafluoroethyl (—C₂F5).

“Alkoxy” refers to an alkyl group, as defined herein, that is single bonded to an oxygen atom. The attachment point of an alkoxy radical to a molecule is through the oxygen atom. An alkoxy radical may be depicted as alkyl-O— or O(C₁₋ₓalkyl). Alkoxy groups may contain, but are not limited to, 1 to 8 carbon atoms (“C₁-C₅ alkoxy”), 1 to 6 carbon atoms (“C₁-C₆ alkoxy”), 1 to 4 carbon atoms (“C₁-C₄ alkoxy”), or 1 to 3 carbon atoms (“C₁-C₃ alkoxy”). Alkoxy groups include, but are not limited to, methoxy, ethoxy, n-propoxy, isobutoxy, and the like.

“Alkoxyalkyl” refers to an alkyl group, as defined herein, that is substituted by an alkoxy group, as defined herein. “Alkoxyalkyl” may be depicted as C₁₋ₓalkylene-O—C₁₋ₓalkyl. Examples include, but are not limited to, CH₃OCH₂— and CH₃CH₂OCH₂—.

“Cycloalkyl” refers to a fully saturated hydrocarbon ring system that has the specified number of carbon atoms, which may be a monocyclic, bridged or fused bicyclic or polycyclic ring system that is connected to the base molecule through a carbon atom of the cycloalkyl ring. Cycloalkyl groups may contain, but are not limited to, 3 to 12 carbon atoms (“C₃-C₁₂ cycloalkyl”), 3 to 8 carbon atoms (“C₃-C₈ cycloalkyl”), 3 to 6 carbon atoms (“C₃-C₆ cycloalkyl”), 3 to 5 carbon atoms (“C₃-C₅ cycloalkyl”) or 3 to 4 carbon atoms (“C₃-C₄ cycloalkyl”). Representative cycloalkyl rings include cyclopropyl, cyclobutyl, cyclopentyl, cyclohexyl, cycloheptyl, and cyclooctyl. Polycyclic cycloalkyl groups include, for example, adamantanyl, 1,2-dihydronaphthalenyl, 1,4-dihydronaphthalenyl, tetraenyl, decalinyl, 3,4-dihydronaphthalenyl-1(2H)-one, spiro[2.2]pentyl, norbornyl, and bicyclo[1.1.1]pentyl. Cycloalkyl groups may be optionally substituted, unsubstituted or substituted, as further defined herein.

“Cycloalkoxy” refers to a cycloalkyl group, as defined herein, that is single bonded to an oxygen atom. The attachment point of a cycloalkoxy radical to a molecule is through the oxygen atom. A cycloalkoxy radical may be depicted as cycloalkyl-O— or OC₁₋ₓcycloalkyl. Cycloalkoxy groups may contain, but are not limited to, 3 to 8 carbon atoms (“C₃-C₈ cycloalkoxy”), 3 to 6 carbon atoms (“C₃-C₆ cycloalkoxy”), and 3 to 4 carbon atoms (“C₃-C₄ cycloalkoxy”).

Representative cycloalkyl rings include cyclopropyl, cyclobutyl, cyclopentyl, cyclohexyl, cycloheptyl, and cyclooctyl. Polycyclic cycloalkyl groups include, for example, adamantanyl, 1,2-dihydronaphthalenyl, 1,4-dihydronaphthalenyl, tetraenyl, decalinyl, 3,4-dihydronaphthalenyl-1(2H)-one, spiro[2.2]pentyl, norbornyl, and bicyclo[1.1.1]pentyl.

“Heterocycloalkyl” refers to a fully saturated ring system containing the specified number of ring atoms and containing at least one heteroatom selected from N, O and S as a ring member, where ring S atoms are optionally substituted by one or two oxo groups (i.e., S(O)_{q}, where q is 0, 1 or 2) and where the heterocycloalkyl ring is connected to the base molecule via a ring atom, which may be C or N. Heterocycloalkyl rings include rings which are spirocyclic, bridged, or fused to one or more other heterocycloalkyl or carbocyclic rings, where such spirocyclic, bridged, or fused rings may themselves be saturated, partially unsaturated or aromatic to the extent unsaturation or aromaticity makes chemical sense, provided the point of attachment to the base molecule is an atom of the heterocycloalkyl portion of the ring system. Heterocycloalkyl rings may contain 1 to 4 heteroatoms selected from N, O, and S(O)_{q} as ring members, or 1 to 2 ring heteroatoms, provided that such heterocycloalkyl rings do not contain two contiguous oxygen or sulfur atoms. Heterocycloalkyl rings may be optionally substituted, unsubstituted or substituted, as further defined herein. Such substituents may be present on the heterocyclic ring attached to the base molecule, or on a spirocyclic, bridged or fused ring attached thereto. Heterocycloalkyl rings may include, but are not limited to, 3-8 membered heterocycloalkyl groups, for example 4-7 or 4-6 membered heterocycloalkyl groups, in accordance with the definition herein. Illustrative examples of heterocycloalkyl rings include, but are not limited to a monovalent radical of oxirane (oxiranyl), thiirane (thiiranyl), aziridine (aziridinyl), oxetane (oxetanyl), thietane (thietanyl), azetidine (azetidinyl), tetrahydrofuran (tetrahydrofuranyl), tetrahydrothiophene (tetrahydrothiophenyl), pyrrolidine (pyrrolidinyl), tetrahydropyran (tetrahydropyranyl), tetrahydrothiopyran (tetrahydrothiopyranyl), piperidine (piperidinyl), 1,4-dioxane (1,4-dioxanyl), 1,4-oxathiarane (1,4-oxathiaranyl), morpholine (morpholinyl), 1,4-dithiane (1,4-dithianyl), piperazine (piperazinyl), thiomorpholine (thiomorpholinyl), oxepane (oxepanyl), thiepane (thiepanyl), azepane (azepanyl), 1,4-dioxepane (1,4-dioxepanyl), 1,4-oxathiepane (1,4-oxathiepanyl), 1,4-oxaazepane (1,4-oxaazepanyl), 1,4-thiazepane (1,4-thiazapanyl), 1,4-diazepane (1,4-diazepanyl), or 1,4-dithepane (1,4-dithiepanyl). Illustrative examples of bridged and fused heterocycloalkyl groups include, but are not limited to a monovalent radical of 1-oxa-5-azabicyclo-[2.2.1]heptane, 3-oxa-8-azabicyclo-[3.2.1]octane, 3-azabicyclo-[3.1.0]hexane, or 2-azabicyclo-[3.1.0]hexane.

“Aryl” refers to monocyclic, bicyclic (e.g., biaryl, fused) or polycyclic ring systems that contain the specified number of ring atoms, in which all carbon atoms in the ring are of sp² hybridization and in which the pi electrons are in conjugation. Aryl groups may contain, but are not limited to, 6 to 20 carbon atoms (“C₆-C₂₀ aryl”), 6 to 14 carbon atoms (“C₆-C₁₄aryl”), 6 to 12 carbon atoms (“C₆-C₁₂ aryl”), or 6 to 10 carbon atoms (“C₆-C₁₀ aryl”). Fused aryl groups may include an aryl ring (e.g., a phenyl ring) fused to another aryl ring. Examples include, but are not limited to, phenyl, biphenyl, naphthyl, anthracenyl, phenanthrenyl, indanyl, and indenyl. Aryl groups may be optionally substituted, unsubstituted or substituted, as further defined herein.

Similarly, “heteroaryl” or “heteroaromatic” refer to monocyclic, bicyclic (e.g., heterobiaryl, fused) or polycyclic ring systems that contain the specified number of ring atoms and include at least one heteroatom selected from N, O and S as a ring member in a ring in which all carbon atoms in the ring are of sp² hybridization and in which the pi electrons are in conjugation. Heteroaryl groups may contain, but are not limited to, 5 to 20 ring atoms (“5-20 membered heteroaryl”), 5 to 14 ring atoms (“5-14 membered heteroaryl”), 5 to 12 ring atoms (“5-12 membered heteroaryl”), 5 to 10 ring atoms (“5-10 membered heteroaryl”), 5 to 9 ring atoms (“5-9 membered heteroaryl”), or 5 to 6 ring atoms (“5-6 membered heteroaryl”). Heteroaryl rings are attached to the base molecule via a ring atom of the heteroaromatic ring. Thus, either 5- or 6-membered heteroaryl rings, alone or in a fused structure, may be attached to the base molecule via a ring C or N atom. Examples of heteroaryl groups include, but are not limited to, pyrrolyl, furanyl, thiophenyl, pyrazolyl, imidazolyl, isoxazolyl, oxazolyl, isothiazolyl, thiazolyl, triazolyl, oxadiazolyl, thiadiazolyl, tetrazolyl, pyridinyl, pyridizinyl, pyrimidinyl, pyrazinyl, benzofuranyl, benzothiophenyl, indolyl, benzimidazolyl, indazolyl, quinolinyl, isoquinolinyl, purinyl, triazinyl, naphthyridinyl, cinnolinyl, quinazolinyl, quinoxalinyl and carbazolyl. Examples of 5- or 6-membered heteroaryl groups include, but are not limited to, pyrrolyl, furanyl, thiophenyl, pyrazolyl, imidazolyl, isoxazolyl, oxazolyl, isothiazolyl, thiazolyl, triazolyl, pyridinyl, pyrimidinyl, pyrazinyl and pyridazinyl rings. Heteroaryl groups may be optionally substituted, unsubstituted or substituted, as further defined herein. Illustrative examples of monocyclic heteroaryl groups include, but are not limited to a monovalent radical of pyrrole (pyrrolyl), furan (furanyl), thiophene (thiophenyl), pyrazole (pyrazolyl), imidazole (imidazolyl), isoxazole (isoxazolyl), oxazole (oxazolyl), isothiazole (isothiazolyl), thiazolyl (thiazolyl), 1,2,3-triazole (1,2,3-triazolyl), 1,3,4-triazole (1,3,4-triazolyl), 1-oxa-2,3-diazole (1-oxa-2,3-diazolyl), 1-oxa-2,4-diazole (1-oxa-2,4-diazolyl), 1-oxa-2,5-diazole (1-oxa-2,5-diazolyl), 1-oxa-3,4-diazole (1-oxa-3,4-diazolyl), 1-thia-2,3-diazole (1-thia-2,3-diazolyl), 1-thia-2,4-diazole (1-thia-2,4-diazolyl), 1-thia-2,5-diazole (1-thia-2,5-diazolyl), 1-thia-3,4-diazole (1-thia-3,4-diazolyl), tetrazole (tetrazolyl), pyridine (pyridinyl), pyridazine (pyridazinyl), pyrimidine (pyrimidinyl), or pyrazine (pyrazinyl).

Illustrative examples of fused ring heteroaryl groups include, but are not limited to benzofuran (benzofuranyl), benzothiophene (benzothiophenyl), indole (indolyl), benzimidazole (benzimidazolyl), indazole (indazolyl), benzotriazole (benzotriazolyl), pyrrolo[2,3-b]pyridine (pyrrolo[2,3-b]pyridinyl), pyrrolo[2,3-c]pyridine (pyrrolo[2,3-c]pyridinyl), pyrrolo[3,2-c]pyridine (pyrrolo[3,2-c]pyridinyl), pyrrolo[3,2-b]pyridine (pyrrolo[3,2-b]pyridinyl), imidazo[4,5-b]pyridine (imidazo[4,5-b]pyridinyl), imidazo[4,5-c]pyridine (imidazo[4,5-c]pyridinyl), pyrazolo[4,3-d]pyridine (pyrazolo[4,3-d]pyridinyl), pyrazolo[4,3-c]pyridine (pyrazolo[4,3-c]pyridinyl), pyrazolo[3,4-c]pyridine (pyrazolo[3,4-c]pyridinyl), pyrazolo[3,4-b]pyridine (pyrazolo[3,4-b]pyridinyl), isoindole (isoindolyl), indazole (indazolyl), purine (purinyl), indolizine (indolizinyl), imidazo[1,2-a]pyridine (imidazo[1,2-a]pyridinyl), imidazo[1,5-a]pyridine (imidazo[1,5-a]pyridinyl), pyrazolo[1,5-a]pyridine (pyrazolo[1,5-a]pyridinyl), pyrrolo[1,2-b]pyridazine (pyrrolo[1,2-b]pyridazinyl), imidazo[1,2-c]pyrimidine (imidazo[1,2-c]pyrimidinyl), quinoline (quinolinyl), isoquinoline (isoquinolinyl), cinnoline (cinnolinyl), quinazoline (azaquinazoline), quinoxaline (quinoxalinyl), phthalazine (phthalazinyl), 1,6-naphthyridine (1,6-naphthyridinyl), 1,7-naphthyridine (1,7-naphthyridinyl), 1,8-naphthyridine (1,8-naphthyridinyl), 1,5-naphthyridine (1,5-naphthyridinyl), 2,6-naphthyridine (2,6-naphthyridinyl), 2,7-naphthyridine (2,7-naphthyridinyl), pyrido[3,2-d]pyrimidine (pyrido[3,2-d]pyrimidinyl), pyrido[4,3-d]pyrimidine (pyrido[4,3-d]pyrimidinyl), pyrido[3,4-d]pyrimidine (pyrido[3,4-d]pyrimidinyl), pyrido[2,3-d]pyrimidine (pyrido[2,3-d]pyrimidinyl), pyrido[2,3-b]pyrazine (pyrido[2,3-b]pyrazinyl), pyrido[3,4-b]pyrazine (pyrido[3,4-b]pyrazinyl), pyrimido[5,4-d]pyrimidine (pyrimido[5,4-d]pyrimidinyl), pyrazino[2,3-b]pyrazine (pyrazino[2,3-b]pyrazinyl), or pyrimido[4,5-d]pyrimidine (pyrimido[4,5-d]pyrimidinyl).

“Amino” refers to a group —NH₂, which is unsubstituted. Where the amino is described as substituted or optionally substituted, the term includes groups of the form —NR^{x}R^{y}, where each of R^{x} and R^{y} are independently defined as further described herein. For example, “alkylamino” refers to a group —NR^{x}R^{y}, wherein one of R^{x} and R^{y} is an alkyl moiety and the other is H, and “dialkylamino” refers to —NR^{x}R^{y} wherein both of R^{x} and R^{y} are alkyl moieties, where the alkyl moieties have the specified number of carbon atoms (e.g., —NH(C₁-C₄ alkyl) or —N(C₁-C₄ alkyl)₂).

The term “alkylamino” or “aminoalkyl” refer to a radical of the formula —NHR^{x} or —NR^{x}R^{y}, wherein each R^{x} and R^{y} are independently H, an alkyl group, or an alkylene group. For example, “alkylamino” can refer to a group —NR^{x}R^{y}, wherein one of R^{x} and R^{y} is an alkyl moiety and the other is H; and “dialkylamino” can refer to —NR^{x}R^{y}, wherein both of R^{x} and R^{y} are alkyl moieties, where the alkyl moieties have the specified number of carbon atoms (e.g., —NH(C₁-C₄ alkyl) or —N(C₁-C₄ alkyl)₂). In some embodiments, aminoalkyl refers to —NH-alkylene or alkylene-NH-alkylene, wherein each alkyklene is independent substituted or unsubstituted.

The term “pharmaceutically acceptable” means the substance (e.g., the compounds described herein) and any salt thereof, or composition containing the substance or salt of the invention is suitable for administration to a subject or patient.

“Deuterium enrichment factor” as used herein means the ratio between the deuterium abundance and the natural abundance of deuterium, each relative to hydrogen abundance. An atomic position designated as having deuterium typically has a deuterium enrichment factor of, in particular embodiments, at least 1000 (15% deuterium incorporation), at least 2000 (30% deuterium incorporation), at least 3000 (45% deuterium incorporation), at least 3500 (52.5% deuterium incorporation), at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

The term “treating”, “treat” or “treatment” as used herein embraces both preventative, i.e., prophylactic, and palliative treatment, i.e., relieve, alleviate, or slow the progression of the patient's disease (or condition) or any tissue damage associated with the disease.

As used herein, the terms, “subject, “individual” or “patient,” used interchangeably, refer to any animal, including mammals. Mammals according to the invention include canine, feline, bovine, caprine, equine, ovine, porcine, rodents, lagomorphs, primates, humans and the like, and encompass mammals in utero. In an embodiment, humans are suitable subjects. Human subjects may be of any gender and at any stage of development.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which may include one or more of the following:
(1) preventing the disease; for example, preventing a disease, condition or disorder in an individual that may be predisposed to the disease, condition or disorder but does not yet experience or display the pathology or symptomatology of the disease;
(2) inhibiting the disease; for example, inhibiting a disease, condition or disorder in an individual that is experiencing or displaying the pathology or symptomatology of the disease, condition or disorder (i.e., arresting (or slowing) further development of the pathology or symptomatology or both); and
(3) ameliorating the disease; for example, ameliorating a disease, condition or disorder in an individual that is experiencing or displaying the pathology or symptomatology of the disease, condition or disorder (i.e., reversing the pathology or symptomatology or both).

As used herein, the term D10 means that about 10% of particles in a composition are smaller than a specified size. D10 can also be represented using statistical symbols, for example, DV(0.1) or DV0.1, wherein V means total volume. D10 is represents a small particle size.

As used herein, the term D50 means that about 50% of particles in a composition are smaller than a specified size. D50 can also be represented using statistical symbols, for example, DV(0.5) or DV0.5, wherein V means total volume. D50 is represents a medium particle size. D50 is the median particle distribution.

As used herein, the term D90 means that about 90% of particles in a composition are smaller than a specified size. D90 can also be represented using statistical symbols, for example, DV(0.9) or DV0.9, wherein V means total volume. D10 is represents a large particle size. Particle sizes that exceed a range of D10 to D90 can be ignored because of the small amount of particles.

As used herein, the term T80 refers to the time for 80% of an active pharmaceutical ingredient (API) to be released from an extended release formulation in Fasted State Simulated Intestinal Fluid (FaSSIF), which is a descriptor of in vitro release performance. Tests performed in FaSSIF dissolution medium can reveal how an oral drug is likely to dissolve and potentially be absorbed in fluid from the upper intestine after drinking a glass of water. FaSSIF contains the same type and level of surfactants (bile salt and phospholipid) present in gastrointestinal fluid.

### Compounds of the Invention

Disclosed herein are AMPK-activating compounds. In some embodiments, the compounds disclosed herein are pan-AMPK-activating compounds.

The present disclosure provides a compound of Formula (I):

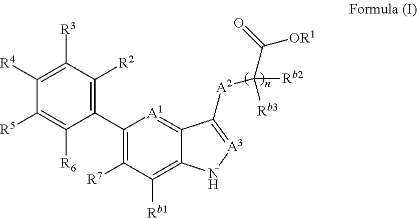

a pharmaceutically acceptable salt, a tautomer, or a pharmaceutically acceptable salt of the tautomer thereof, wherein:
   A¹ is CR⁸, or N;
   A² is CH₂, CHD, CD₂, S, O, or NH;
   A³ is CH, CD, or N;
   R¹ is H, D, C₁₋₈alkyl, C₃₋₆cycloalkyl, or 4-6 membered heterocycloalkyl, each of which is optionally substituted;
   R², R³, R⁵, and R⁶ are each independently H, D, OH, or halogen;
   R⁴ is monocyclic aryl, bicyclic aryl, monocyclic heteroaryl, or bicyclic heteroaryl, each of which is optionally substituted with R⁹, R¹⁰, R¹¹, R¹², or R¹³,
      wherein R⁹, R¹⁰, R¹¹, R¹², and R¹³ are each independently H, D, halogen, CN, oxo, C₁₋₈alkyl, C₃₋₆cycloalkyl, C₀₋₆alkylene-OR^{x}, C₁₋₆haloalkylene-OR^{x}, C₀₋₆alkylene(C₀₋₆haloalkyl)NR^{x}R^{y}, C₁₋₆alkylene(C₁₋₆haloalkyl)NRR^{y}, 4-6 membered heterocycloalkyl, C(O)OR^{x}, C₀₋₆alkylene-C(O)NRR^{y}, OC₁₋₃alkylene-heterocycloalkyl, OC₁₋₃alkylene-C(O)NR^{x}R^{y}, O(C₁₋₆alkyl)SO₂NR^{x}NR^{y}, NR^{x}R^{y}, NHSO₂R^{x}, SR^{x}, S—C₁₋₆alkylene-C(O)NR^{x}R^{y}, S(O)R^{x}R^{y}, SO₂R^{x}, SO₂NR^{x}R^{y}, S(O)(NR^{x})R^{y}, S(O)(NR^{x})R^{y}, or SO₂R^{x};
   wherein each R^{x} and R^{y} is independently H, D, C₁₋₆alkyl, C₁₋₆haloalkyl, C₁₋₆alkoxy, C₃₋₆cycloalkyl, C₁₋₆alkylene-amide, OC₀₋₂alkylene-heterocycloalkyl, 4-6 membered heterocycloalkyl, C(O)C₁₋₆alkyl, imino, or C₁₋₆alkylsulfonyl; or R^{x} and R^{y} together with the atoms to which R^{x} and R^{y} are bound can form an optionally substituted ring;
   R⁷ is C₁₋₃alkyl, C₃₋₆cycloalkyl, cyano, or halogen;
   R^{b1}, R^{b2}, and R^{b3} are each independently H or D;
   R³ is H, D, or halogen; and
   n is 0, 1, or 2.

In some embodiments, the compound is of Formula (Ia):

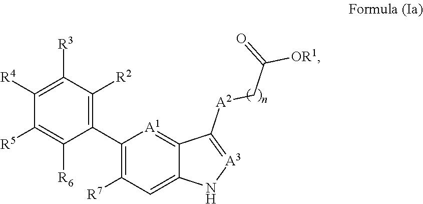

or a pharmaceutically acceptable salt, tautomer, or pharmaceutically acceptable salt of the tautomer thereof.

In some embodiments, A¹ is CR⁸. In some embodiments, A¹ is N. In some embodiments, R³ is H. In some embodiments, R³ is halogen. In a preferred embodiment, A¹ is CH or CF.

In some embodiments, A² is O or NH. In a preferred embodiment, A² is CH₂ or S. In a preferred embodiment, A² is CH₂. In a preferred embodiment, A² is S.

In some embodiments, A³ is CH. In a preferred embodiment, A³ is N. In a preferred embodiment, A¹ is N and A³ is N.

In some embodiments, R¹ is H, D, or C₁₋₈alkyl. In some embodiments, R¹ is C₃₋₆cycloalkyl or 4-6 membered heterocycloalkyl. In some embodiments, R¹ is -6-O-3,4,5-trihydroxy-tetrahydro-2H-pyran-2-carboxylic acid. In a preferred embodiment, R¹ is H.

In some embodiments, R² is H. In some embodiments, R² is halogen. In some embodiments, R² is F. In some embodiments, R² is C. In some embodiments, R³ is H. In some embodiments, R³ is halogen. In some embodiments, R³ is F. In some embodiments, R³ is C. In some embodiments, R⁵ is H. In some embodiments, R⁵ is halogen. In some embodiments, R⁵ is F. In some embodiments, R⁵ is Cl. In some embodiments, R⁶ is H. In some embodiments, R⁶ is halogen. In some embodiments, R⁶ is OH. In some embodiments, R⁶ is F. In some embodiments, R⁶ is Cl. In a preferred embodiment, R² is halogen; and R³, R⁵, and R⁶ are each independently H. In a preferred embodiment, R², R³, R⁵, and R⁶ are each independently H.

In some embodiments, R⁴ is 6-membered monocyclic aryl. In some embodiments, R⁴ is 6-membered monocyclic aryl substituted with 1, 2, or 3 Rₐ. In some embodiments, R⁴ is 5 or 6-membered monocyclic heteroaryl substituted with 1, 2, or 3 Rₐ. In a preferred embodiment, R⁴ is phenyl, wherein R⁹, R¹⁰, R¹¹, R¹², and R¹³ are each independently H, D, Cl, F, CN, C₁₋₃alkyl, C₁₋₆alkylene-OH, C₁₋₆alkylene-OC₁₋₆alkyl, OH, OC₁₋₆alkyl, OC₁₋₆haloalkyl, O(C₁₋₃alkylene)heterocycloalkyl, O(C₁₋₃alkylene)-C(O)NR^{x}R^{y}, C₁₋₃alkylene-NR^{x}R^{y}, C(O)OH, C(O)OC₁₋₃alkyl, C₀₋₂alkylene-C(O)NR^{x}R^{y}, SO₂NR^{x}R^{y}, S(O)(NR^{x})R^{y}, NR^{x}R^{y}, SR^{x}, or SO₂R^{x}. In a preferred embodiment, R⁴ is 6-membered monocyclic heteroaryl, wherein at least one of R⁹, R¹⁰, R¹¹, R¹² and R¹³ is C₁₋₃alkoxy, halogen, hydroxy, or C(O)NH₂.

In some embodiments, R⁷ is C₁₋₃alkyl. In some embodiments, R⁷ is C₃₋₆cycloalkyl. In some embodiments, R⁷ is cyclopropyl. In some embodiments, R⁷ is cyano. In some embodiments, R⁷ is halogen. In a preferred embodiment, R⁷ is Cl. In a preferred embodiment, R⁷ is F.

The present disclosure also provides a compound of Formula (II):

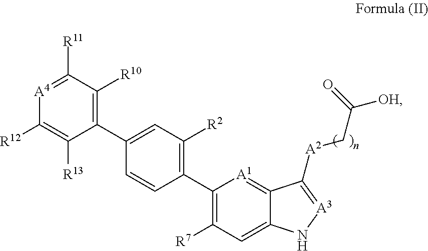

a pharmaceutically acceptable salt, a tautomer, or a pharmaceutically acceptable salt of the tautomer thereof, wherein:
   R² is H, D or halogen;
   R⁷ is Cl or CN;
   A² is CH₂, CHD, CD₂, S, or NH;
   A⁴ is CR⁹ or N;
   R⁹ is H, F, Cl, C₁₋₃alkyl, C₁₋₃alkylene-O—C₁₋₃alkyl, C₁₋₃alkylene-NH₂, COOH, C(O)OC₁₋₃alkyl, C₁₋₃alkylene-C(O)NH₂, C(O)NHC₁₋₃alkyl, C(O)N(C₁₋₃alkyl)₂, C₁₋₆alkylene(C₁₋₆ haloalkyl)NH₂, C₀₋₂alkylene-NH(C(O)C₁₋₃alkyl), OC₁₋₃alkyl, OC₁₋₃haloalkyl, OC₁₋₃alkylene-heterocycloalkyl, OC₁₋₃alkylene-C(O)NH₂, NHSO₂C₁₋₃alkyl, N(C₁₋₃ alkyl)(C(O)C₁₋₃alkyl), SC₁₋₃alkyl, S—C₁₋₃alkylene-C(O)NH₂, SO(NH)C₁₋₃alkyl, SO₂NH₂, or SO₂C₁₋₃alkyl;
   R¹⁰ is H, D, or OH;
   R¹¹ is H, D, halogen, CN, O(C₁₋₃alkyl), or O(C₁₋₃haloalkyl); or R⁹ and R¹¹ together with the carbon atom to which R⁹ and R¹¹ are bound form an optionally substituted ring;
   R¹² is H, OC₁₋₃alkyl, or C₁₋₃alkylene-OH;
   R¹³ is H, F, Cl, or C₁₋₃alkyl; and
   n is 1 or 2.

In some embodiments, R⁷ is CN. In a preferred embodiment, R⁷ is Cl.

In some embodiments, R⁹ is H, F, Cl, C₁₋₃alkyl, C₁₋₃alkylene-O—C₁₋₃alkyl, C₁₋₃alkylene-NH₂, COOH, C(O)OC₁₋₃alkyl, C₁₋₃alkylene-C(O)NH₂, C(O)NHC₁₋₃alkyl, C(O)N(C₁₋₃alkyl)₂, C₁₋₃haloalkylene-NH₂, C₀₋₂alkylene-NH(C(O)C₁₋₃alkyl), OC₁₋₃alkyl, OC₁₋₃haloalkyl, OC₁₋₃alkylene-heterocycloalkyl, OC₁₋₃alkylene-C(O)NH₂, NHSO₂C₁₋₃alkyl, N(C₁₋₃alkyl)(C(O)C₁₋₃alkyl), SC₁₋₃alkyl, S—C₁₋₃alkylene-C(O)NH₂, SO(NH)C₁₋₃alkyl, SO₂NH₂, or SO₂C₁₋₃alkyl. In some embodiments, R⁹ is H, C₁₋₃alkylene-O—C₁₋₃alkyl, C₁₋₃alkylene-NH₂, C₁₋₃alkylene-C(O)NH₂, C₁₋₃alkylene(C₁₋₃haloalkyl)NH₂, or C₀₋₂alkylene-NH(C(O)C₁₋₃alkyl). In some embodiments, R⁹ is NHSO₂C₁₋₃alkyl or N(C₁₋₃alkyl)(C(O)C₁₋₃alkyl). In some embodiments, R⁹ is SC₁₋₃alkyl, S—C₁₋₃alkylene-C(O)NH₂, SO(NH)C₁₋₃alkyl, SO₂NH₂, or SO₂C₁₋₃alkyl. In a preferred embodiment, R⁹ is H. In a preferred embodiment, R⁹ is C(O)NH₂. In a preferred embodiment, R⁹ is C₁₋₃alkylene-NH₂.

In some embodiments, R¹⁰ is H or D. In a preferred embodiment, R¹⁰ is OH.

In some embodiments, R¹¹ is H, F, Cl, or CN. In some embodiments, R¹¹ is O(C₁₋₃alkyl) or O(C₁₋₃haloalkyl). In a preferred embodiment, R¹¹ is H.

The present disclosure further provides a compound of Formula (III):

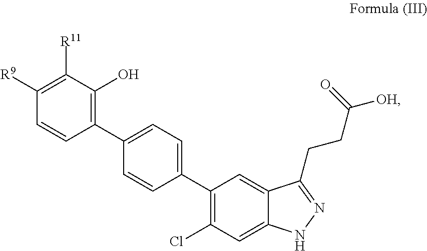

a pharmaceutically acceptable salt, a tautomer, or a pharmaceutically acceptable salt of the tautomer thereof, wherein:
   R⁹ is H, D, halogen, C₁₋₃alkyl, C(O)NH₂, C₁₋₃alkylene-NH₂, C₁₋₃alkylene-O—C₁₋₃alkyl, C(O)OC₁₋₃alkyl, C(O)OH, OC₁₋₃alkyl, OC₁₋₃haloalkyl, —O(C₁₋₃alkyl)SO₂NH₂, C₁₋₆alkylene(C₁₋₆haloalkyl)NH₂, SC₁₋₃alkyl, or —C(O)NR^{x}R^{y}, wherein each R^{x} and R^{y} are independently H or C₁₋₆alkyl; and
   R¹¹ is H, D, halogen, CN, or —O(C₁₋₃alkyl); or R⁹ and R¹¹ together with the carbon atom to which R⁹ and R¹¹ are bound form an optionally substituted ring.

In a preferred embodiment, R⁹ is H or —C(O)NR^{x}R^{y}, wherein each R^{x} and R^{y} are independently H or C₁₋₆alkyl; and R¹¹ is H or —O(C₁₋₃alkyl); or R⁹ and R¹¹ together with the carbon atom to which R⁹ and R¹¹ are bound form an optionally substituted ring.

The present disclosure also provides compounds of Formula (IVa), Formula (IVb), or Formula (IVc):

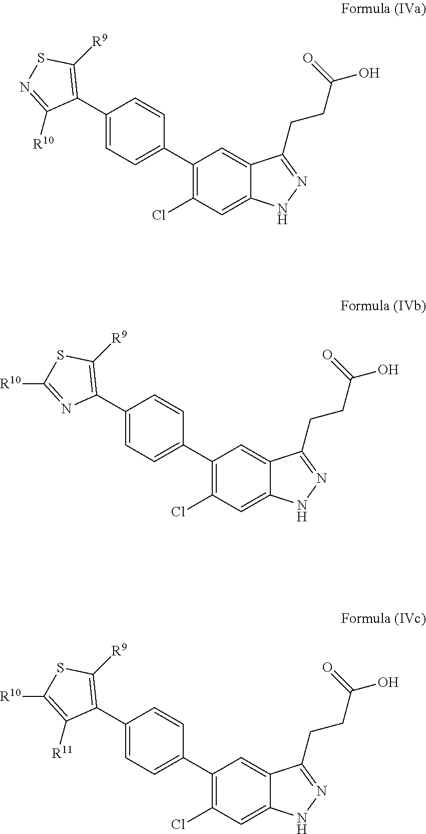

a pharmaceutically acceptable salt, a tautomer, or a pharmaceutically acceptable salt of the tautomer thereof, wherein each R⁹, R¹⁰, and R¹¹ are independently H, C₁₋₃alkyl, C₁₋₃alkylene-OH, or OC₁₋₃alkyl.

In a preferred embodiment, R⁹, R¹⁰, and R¹¹ are each independently H. In a preferred embodiment, R¹⁰ is C₁₋₃alkyl, C₁₋₃alkylene-OH or OC₁₋₃alkyl.

The present disclosure also provides a compound of Formula (V):

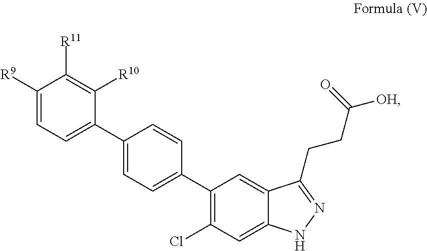

a pharmaceutically acceptable salt, a tautomer, or a pharmaceutically acceptable salt of the tautomer thereof, wherein:
   R¹⁰ is H, D, or OH;
   R⁹ and R¹¹ together with the carbon atom to which R⁹ and R¹¹ are bound form an optionally substituted ring, wherein the ring is 5- or 6-membered heterocycloalkyl or 5- or 6-membered heteroaryl, wherein the ring is optionally substituted with C₁₋₃alkyl, OH, or oxo.

In some embodiments, the ring is 6-membered heterocycloalkyl. In a preferred embodiment, the ring is 5-membered heterocycloalkyl. In some embodiments, the ring is 6-membered heteroaryl. In a preferred embodiment, the ring is 5-membered heteroaryl.

In a preferred embodiment, the ring is substituted with C₁₋₃alkyl. In a preferred embodiment, the ring is substituted with OH. In a preferred embodiment, the ring is substituted with oxo.

In some embodiments, the compound of the disclosure, a pharmaceutically acceptable salt, a tautomer, or a pharmaceutically acceptable salt of the tautomer thereof, is selected from the group consisting of:
3-(6-Chloro-5-(2′-hydroxy-[1,1′-biphenyl]-4-yl)-1H-indazol-3-yl)propanoic acid;
3-(6-chloro-5-(2′-hydroxy-6′-methyl-[1,1′-biphenyl]-4-yl)-1H-indazol-3-yl)propanoic acid;
3-(6-chloro-5-(2′-hydroxy-3′-methoxy-[1,1′-biphenyl]-4-yl)-1H-indazol-3-yl)propanoic acid;
3-(6-chloro-5-(2′-hydroxy-3′-methoxy-6′-methyl-[1,1′-biphenyl]-4-yl)-1H-indazol-3-yl)propanoic acid;
3-(6-chloro-5-(4-(7-hydroxy-2,3-dihydrobenzofuran-6-yl)phenyl)-1H-indazol-3-yl)propanoic acid;
3-(6-chloro-5-(2′-hydroxy-4′-(methoxymethyl)-[1,1′-biphenyl]-4-yl)-1H-indazol-3-yl)propanoic acid;
3-(6-chloro-5-(2′-hydroxy-4′,6′-dimethyl-[1,1′-biphenyl]-4-yl)-1H-indazol-3-yl)propanoic acid;
3-(6-chloro-5-(3′-fluoro-2′-hydroxy-6′-methyl-[1,1′-biphenyl]-4-yl)-1H-indazol-3-yl)propanoic acid;
3-(6-chloro-5-(4′-fluoro-2′-hydroxy-3′-methoxy-[1,1′-biphenyl]-4-yl)-1H-indazol-3-yl)propanoic acid;
3-(6-chloro-5-(4′-(dimethylcarbamoyl)-[1,1′-biphenyl]-4-yl)-1H-indazol-3-yl)propanoic acid;
4-(6-chloro-5-(2′-hydroxy-3′-methoxy-[1,1′-biphenyl]-4-yl)-1H-indazol-3-yl)butanoic acid;
3-(6-chloro-5-(4′-(methylcarbamoyl)-[1,1′-biphenyl]-4-yl)-1H-indazol-3-yl)propanoic acid;
6-((3-(6-chloro-5-(2′-hydroxy-[1,1′-biphenyl]-4-yl)-1H-indazol-3-yl)propanoyl)oxy)-3,4,5-trihydroxytetrahydro-2H-pyran-2-carboxylic acid; and
6-((4′-(3-(2-carboxyethyl)-6-chloro-1H-indazol-5-yl)-[1,1′-biphenyl]-2-yl)oxy)-3,4,5-trihydroxytetrahydro-2H-pyran-2-carboxylic acid.

In a preferred embodiment, the compound is 3-[6-chloro-5-(2′-hydroxy[1,1′-biphenyl]-4-yl)-1H-indazol-3-yl]propanoic acid, a pharmaceutically acceptable salt, a tautomer, or a pharmaceutically acceptable salt of the tautomer thereof. In a preferred embodiment, the compound is 3-[6-chloro-5-(2′-hydroxy[1,1′-biphenyl]-4-yl)-1H-indazol-3-yl]propanoic acid. In a preferred embodiment, the compound has the structure:

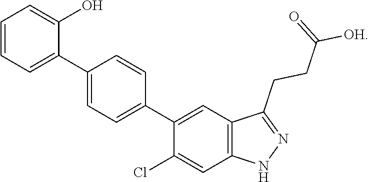 [SMILES: O=C(O)CCc1n[nH]c2cc(Cl)c(-c3ccc(-c4ccccc4O)cc3)cc12]

Any compound herein can be purified. A compound herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

### Pharmaceutically Acceptable Salts

Salts encompassed within the term “pharmaceutically acceptable salts” refer to the compounds of this disclosure which are generally prepared by reacting the free base or free acid with a suitable organic or inorganic acid, or a suitable organic or inorganic base, respectively, to provide a salt of the compound of the disclosure that is suitable for administration to a subject or patient.

In addition, the compounds of Formula I may also include other salts of such compounds which are not necessarily pharmaceutically acceptable salts, which may be useful as intermediates for one or more of the following: 1) preparing compounds of Formula I, Ia, II, III, IVa-c; 2) purifying compounds of Formula I, Ia, II, III, IVa-c; 3) separating enantiomers of compounds of Formula I, Ia, II, III, IVa-c; or 4) separating diastereomers of compounds of Formula I, Ia, II, III, IVa-c.

Suitable acid addition salts are formed from acids which form non-toxic salts. Examples include, but are not limited to, acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate, 1,5-naphathalenedisulfonic acid and xinofoate salts.

Suitable base salts are formed from bases which form non-toxic salts. Examples include, but are not limited to aluminum, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulfate and hemicalcium salts.

For a review on suitable salts, see PAULEKUHN, G. S., et al., “Trends in Active Pharmaceutical Ingredient Salt Selection Based on Analysis of the Orange Book Database,” Journal of Medicinal Chemistry, 2007, 50(26):6665-6672.

Pharmaceutically acceptable salts of compounds of the disclosure may be prepared by methods well known to one skilled in the art, including but not limited to the following procedures
(i) by reacting a compound of the disclosure with the desired acid or base;
(ii) by removing an acid- or base-labile protecting group from a suitable precursor of a compound of the disclosure or by ring-opening a suitable cyclic precursor, for example, a lactone or lactam, using the desired acid or base; or
(iii) by converting one salt of a compound of the disclosure to another. This may be accomplished by reaction with an appropriate acid or base or by means of a suitable ion exchange procedure.

These procedures are typically carried out in solution. The resulting salt may precipitate out and be collected by filtration or may be recovered by evaporation of the solvent.

### Solvates

The compounds of the disclosure, and pharmaceutically acceptable salts thereof, may exist in unsolvated and solvated forms. The term ‘solvate’ is used herein to describe a molecular complex comprising the compound of the disclosure, or a pharmaceutically acceptable salt thereof, and one or more pharmaceutically acceptable solvent molecules, for example, ethanol. The term ‘hydrate’ is employed when said solvent is water.

In addition, the compounds of Formula I, Ia, II, III, IVa-c may also include other solvates of such compounds which are not necessarily pharmaceutically acceptable solvates, which may be useful as intermediates for one or more of the following: 1) preparing compounds of Formula I, Ia, II, III, IVa-c; 2) purifying compounds of Formula I, Ia, II, III, IVa-c; 3) separating enantiomers of compounds of Formula I, Ia, II, III, IVa-c; or 4) separating diastereomers of compounds of Formula I, Ia, II, III, IVa-c.

A currently accepted classification system for organic hydrates is one that defines isolated site, channel, or metal-ion coordinated hydrates—see BRITTAIN, H. G. Polymorphism in Pharmaceutical Solids. 2^{nd} Ed. CRC Press, 2009. Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex may have a well-defined stoichiometry independent of humidity. When, however, the solvent or water is weakly bound, as in channel solvates and hygroscopic compounds, the water/solvent content may be dependent on humidity and drying conditions. In such cases, non-stoichiometry will be the norm.

### Complexes

Also included within the scope of the disclosure are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, for example, hydrogen bonded complex (cocrystal) may be formed with either a neutral molecule or with a salt. Co-crystals may be prepared by melt crystallization, by recrystallization from solvents, or by physically grinding the components together—see ALMARSSON, O. and ZAWOROTKO, M. J., “Crystal engineering of the composition of pharmaceutical phases. Do pharmaceutical co-crystals represent a new path to improved medicines?,” Chemical Communications, 2004, 17:1889-1896. For a general review of multi-component complexes, see HALEBLIAN, J. K., “Characterization of habits and crystalline modification of solids and their pharmaceutical applications,” Journal of Pharmaceutical Sciences, 1975, 64(8):1269-1288.

### Solid Form

The compounds of the disclosure may exist in a continuum of solid states ranging from amorphous to crystalline. The term ‘amorphous’ refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

The compounds of the disclosure may also exist in a mesomorphic state (mesophase or liquid crystal) when subjected to suitable conditions. The mesomorphic state is intermediate between the true crystalline state and the true liquid state (either melt or solution) and consists of two dimensional order on the molecular level. Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see HARTSHORNE, N. H. and STUART, A., Crystals and the Polarizing Microscope. 4^{th} Ed. London, Edward Arnold, 1970.

In a preferred embodiment, a compound of the disclosure is a crystalline of the compound. In a preferred embodiment, the compound is crystalline 3-[6-Chloro-5-(2′-hydroxy[1,1-biphenyl]-4-yl)-1H-indazol-3-yl]propanoic acid, a pharmaceutically acceptable salt, a tautomer, or a pharmaceutically acceptable salt of the tautomer thereof. In a preferred embodiment, the compound is crystalline 3-[6-Chloro-5-(2′-hydroxy[1,1′-biphenyl]-4-yl)-1H-indazol-3-yl]propanoic acid, a pharmaceutically acceptable salt, a tautomer, or a pharmaceutically acceptable salt of the tautomer thereof, having an X-ray powder diffraction pattern comprising diffraction peaks of about 12.6±0.2, about 18.8±0.2, about 19.7±0.2, and about 24.4±0.2 degrees two theta.

### Stereoisomers

Compounds of the disclosure may exist as two or more stereoisomers. Stereoisomers of the compounds may include cis and trans isomers (geometric isomers), optical isomers such as R and S enantiomers, diastereomers, rotational isomers, atropisomers, and conformational isomers. For example, compounds of the disclosure containing one or more asymmetric carbon atoms may exist as two or more stereoisomers. Cis/trans isomers may also exist for saturated rings.

The pharmaceutically acceptable salts of compounds of the disclosure may also contain a counterion which is optically active (e.g., d-lactate or I-lysine) or racemic (e.g., dl-tartrate or dl-arginine).

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Alternatively, the racemate (or a racemic precursor) may be reacted with a suitable optically active compound, for example, an alcohol, or, in the case where a compound of the disclosure contains an acidic or basic moiety, a base or acid such as 1-phenylethylamine or tartaric acid. The resulting diastereomeric mixture may be separated by chromatography, fractional crystallization, or by using both of said techniques, and one or both of the diastereoisomers converted to the corresponding pure enantiomer(s) by means well known to a skilled person. Chiral compounds of the disclosure (and chiral precursors thereof) may be obtained in enantiomerically-enriched form using chromatography, typically HPLC Concentration of the eluate affords the enriched mixture. Chiral chromatography using sub- and supercritical fluids may be employed. Methods for chiral chromatography useful in some embodiments of the present disclosure are known in the art (see, for example, SMITH, R. M., Supercritical Fluid Chromatography with Packed Columns. 1st Ed. RSC Chromatography Monographs, 1988.

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two crystal forms are produced in equimolar amounts each comprising a single enantiomer. While both of the crystal forms present in a racemic mixture have identical physical properties, they may have different physical properties compared to the true racemate. Racemic mixtures may be separated by conventional techniques known to those skilled in the art—see, for example, ELIEL, E. L. and WILEN, S. H., Stereochemistry of Organic Compounds. 1^{st} Ed. New York, Wiley, 1994.

### Tautomerism

Where structural isomers are interconvertible via a low energy barrier, tautomeric isomerism (‘tautomerism’) may occur. This may take the form of proton tautomerism in compounds of the invention containing, for example, an imino/amino, keto/enol, or oxime/nitroso group, lactam/lactim or so-called valence tautomerism in compounds which contain an aromatic moiety. It follows that a single compound may exhibit more than one type of isomerism.

It must be emphasized that while, for conciseness, the compounds of the invention have been drawn herein in a single tautomeric form, all possible tautomeric forms are included within the scope of the invention.

### Isotopes

The present disclosure includes all pharmaceutically acceptable isotopically-labeled compounds of the disclosure wherein one or more atoms are replaced by atoms having the same atomic number, but an atomic mass or mass number different from the atomic mass or mass number which predominates in nature.

Examples of isotopes suitable for inclusion in the compounds of the disclosure may include isotopes of hydrogen, such as ²H (D, deuterium) and ³H (T, tritium), carbon, such as ¹¹C, ¹³C and ¹⁴C, chlorine, such as ³⁶Cl, fluorine, such as ¹⁸F, iodine, such as ¹²³I and ¹²⁵I nitrogen, such as ¹³N and ¹⁵N, oxygen, such as ¹⁵O, ¹⁷O and ¹⁸O, phosphorus, such as ³²P, and sulfur, such as ³⁵S.

Certain isotopically-labelled compounds of the disclosure, for example those incorporating a radioactive isotope, are useful in one or both of drug or substrate tissue distribution studies. The radioactive isotopes, such as, tritium and ¹⁴C are particularly useful for this purpose in view of their ease of incorporation and ready means of detection. Substitution with positron emitting isotopes, such as, ¹¹C ¹⁸F, ¹⁵O and ¹³N, may be useful in Positron Emission Topography (PET) studies for examining substrate receptor occupancy. Substitution with deuterium may afford certain therapeutic advantages resulting from greater metabolic stability, for example increased in vivo half-life, reduced dosage requirements, reduced CYP450 inhibition (competitive or time dependent), or an improvement in therapeutic index or tolerability.

In some embodiments, the disclosure provides deuterium-labeled (or deuterated) compounds and salts, where the formula and variables of such compounds and salts are each and independently as described herein. “Deuterated” means that at least one of the atoms in the compound is deuterium in an abundance that is greater than the natural abundance of deuterium (typically approximately 0.015%). A skilled artisan recognized that in chemical compounds with a hydrogen atom, the hydrogen atom actually represents a mixture of H and D, with about 0.015% being D. The concentration of the deuterium incorporated into the deuterium-labeled compounds and salt of the disclosure may be defined by the deuterium enrichment factor. It is understood that one or more deuterium may exchange with hydrogen under physiological conditions.

In some embodiments, one or more hydrogen atoms on certain metabolic sites on the compounds of the disclosure are deuterated. Isotopically-labeled compounds of the disclosure may generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the disclosure include those wherein the solvent of crystallization may be isotopically substituted, e.g., D₂O, d₆-acetone, d₆-DMSO.

In some embodiments, the deuterium compound is selected from any one of the compounds set forth in Table 5 shown in the Examples section. In some embodiments, one or more hydrogen atoms on certain metabolic sites on the compounds of the invention are deuterated. In some embodiments, the deuterium compound is selected from the group consisting of:

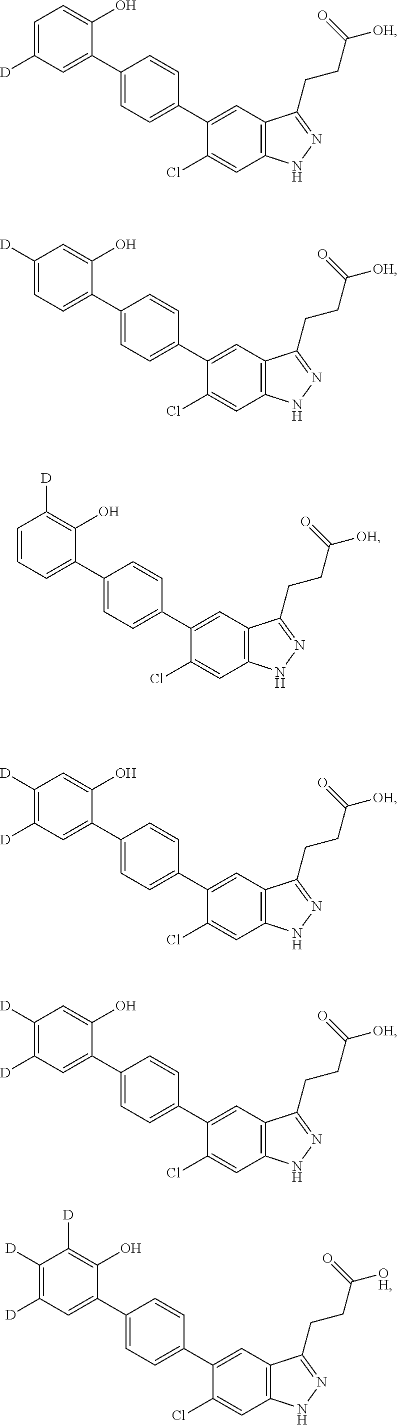 [SMILES: [2H]c1ccc(O)c(-c2ccc(-c3cc4c(CCC(=O)O)n[nH]c4cc3Cl)cc2)c1 ; [2H]c1ccc(-c2ccc(-c3cc4c(CCC(=O)O)n[nH]c4cc3Cl)cc2)c(O)c1 ; [2H]c1cccc(-c2ccc(-c3cc4c(CCC(=O)O)n[nH]c4cc3Cl)cc2)c1O ; [2H]c1cc(O)c(-c2ccc(-c3cc4c(CCC(=O)O)n[nH]c4cc3Cl)cc2)cc1[2H] ; [2H]c1cc(O)c(-c2ccc(-c3cc4c(CCC(=O)O)n[nH]c4cc3Cl)cc2)cc1[2H] ; [2H]c1cc(-c2ccc(-c3cc4c(CCC(=O)O)n[nH]c4cc3Cl)cc2)c(O)c([2H])c1[2H]]

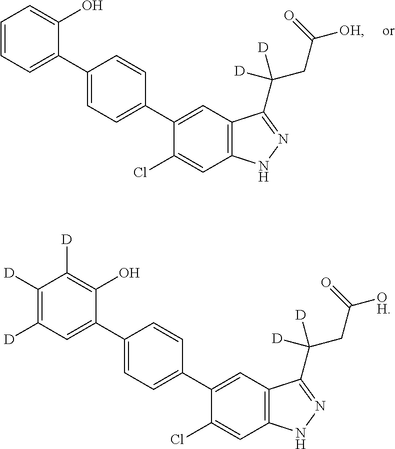 [SMILES: [2H]C([2H])(CC(=O)O)c1n[nH]c2cc(Cl)c(-c3ccc(-c4ccccc4O)cc3)cc12 ; [2H]c1cc(-c2ccc(-c3cc4c(C([2H])([2H])CC(=O)O)n[nH]c4cc3Cl)cc2)c(O)c([2H])c1[2H]]

Isotopically-labeled compounds of the invention may generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the invention include those wherein the solvent of crystallization may be isotopically substituted, e.g., D₂O, d₆-acetone, d₆-DMSO.

### Prodrugs

A compound of the disclosure may be administered in the form of a prodrug. Thus, certain derivatives of a compound of the disclosure which may have little or no pharmacological activity themselves may, when administered into or onto the body, be converted into a compound of the disclosure having the desired activity, for example by hydrolytic cleavage, particularly hydrolytic cleavage promoted by an esterase or peptidase enzyme. Such derivatives are referred to as ‘prodrugs’. Further information on the use of prodrugs may be found in RAUTIO, J., et al., “The expanding role of prodrugs in contemporary drug design and development,” Nature Reviews Drug Discovery, 2018, 17(8):559-587.

Prodrugs in accordance with the disclosure may, for example, be produced by replacing appropriate functionalities present in compounds of the disclosure with certain moieties known to those skilled in the art as ‘pro-moieties’ as described, for example, in BUNDGAARD, H., Design of Prodrugs. New York, Elsevier, 1985.

Thus, a prodrug in accordance with the disclosure may be (a) an ester or amide derivative of a carboxylic acid when present in a compound of the disclosure; (b) an ester, carbonate, carbamate, phosphate or ether derivative of a hydroxyl group when present in a compound of the disclosure; (c) an amide, imine, carbamate or amine derivative of an amino group when present in a compound of the disclosure; (d) a thioester, thiocarbonate, thiocarbamate or sulfide derivatives of a thiol group when present in a compound of the disclosure; or (e) an oxime or imine derivative of a carbonyl group when present in a compound of the disclosure.

Some specific examples of prodrugs in accordance with the disclosure include:
(i) when a compound of the disclosure contains a carboxylic acid functionality (—COOH), an ester thereof, such as a compound wherein the hydrogen of the carboxylic acid functionality of the compound is replaced by C₁-C₈ alkyl (e.g., ethyl) or (C₁-C₅ alkyl)C(═O)OCH₂— (e.g., ^{t}BuC(═O)OCH₂—);
(ii) when a compound of the disclosure contains an alcohol functionality (—OH), an ester thereof, such as a compound wherein the hydrogen of the alcohol functionality of the compound is replaced by —CO(C₁-C₈ alkyl) (e.g., methylcarbonyl) or the alcohol is esterified with an amino acid;
(iii) when a compound of the disclosure contains an alcohol functionality (—OH), an ether thereof, such as a compound wherein the hydrogen of the alcohol functionality of the compound is replaced by (C₁-C₅ alkyl)C(═O)OCH₂— or —CH₂OP(═O)(OH)₂;
(iv) when a compound of the disclosure contains an alcohol functionality (—OH), a phosphate thereof, such as a compound wherein the hydrogen of the alcohol functionality of the compound is replaced by —P(═O)(OH)₂ or —P(═O)(O—Na⁺)₂ or —P(═O)(O-)₂Ca2+
(v) when a compound of the disclosure contains a primary or secondary amino functionality (—NH₂ or —NHR where R #H), an amide thereof, for example, a compound wherein, as the case may be, one or both hydrogens of the amino functionality of the compound is/are replaced by (C₁-C₁₀)alkanoyl, —COCH₂NH₂ or the amino group is derivatized with an amino acid;
(vi) when a compound of the disclosure contains a primary or secondary amino functionality (—NH₂ or —NHR where R #H), an amine thereof, for example, a compound wherein, as the case may be, one or both hydrogens of the amino functionality of the compound is/are replaced by —CH₂OP(═O)(OH)₂.

Certain compounds of the disclosure may themselves act as prodrugs of other compounds the disclosure It is also possible for two compounds of the disclosure to be joined together in the form of a prodrug. In certain circumstances, a prodrug of a compound of the disclosure may be created by internally linking two functional groups in a compound of the disclosure, for instance by forming a lactone.

### Metabolites

Also included within the scope of the disclosure are active metabolites of compounds of the disclosure, that is, compounds formed in vivo upon administration of the drug, often by oxidation or dealkylation. Some examples of metabolites in accordance with the disclosure include, but are not limited to,
(i) where the compound of the disclosure contains an alkyl group, a hydroxyalkyl derivative thereof (—CH→—COH):
(ii) where the compound of the disclosure contains an alkoxy group, a hydroxy derivative thereof (—OR→—OH);
(iii) where the compound of the disclosure contains a tertiary amino group, a secondary amino derivative thereof (—NRR′→—NHR or —NHR′);
(iv) where the compound of the disclosure contains a secondary amino group, a primary derivative thereof (—NHR→—NH₂);
(v) where the compound of the disclosure contains a phenyl moiety, a phenol derivative thereof (—Ph→—PhOH);
(vi) where the compound of the disclosure contains an amide group, a carboxylic acid derivative thereof (—CONH₂∛COOH); and
(vii) where the compound contains a hydroxy or carboxylic acid group, the compound may be metabolized by conjugation, for example with glucuronic acid to form a glucuronide.

Other routes of conjugative metabolism exist. These pathways are frequently known as Phase 2 metabolism and include, for example, sulfation or acetylation. Other functional groups, such as NH groups, may also be subject to conjugation.

In a preferred embodiment, a metabolite of a compound disclosed herein can comprise an O-3,4,5-trihydroxytetrahydro-2H-pyran-2-carboxylic acid moiety. In a preferred embodiment, the metabolite is 6-((3-(6-chloro-5-(2′-hydroxy-[1,1′-biphenyl]-4-yl)-1H-indazol-3-yl)propanoyl)oxy)-3,4,5-trihydroxytetrahydro-2H-pyran-2-carboxylic acid, a pharmaceutically acceptable salt, a tautomer, or a pharmaceutically acceptable salt of the tautomer thereof. In a preferred embodiment, the metabolite has the structure:

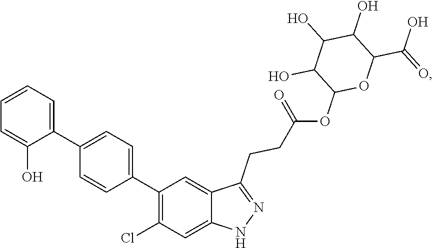 [SMILES: O=C(CCc1n[nH]c2cc(Cl)c(-c3ccc(-c4ccccc4O)cc3)cc12)OC1OC(C(=O)O)C(O)C(O)C1O]

a pharmaceutically acceptable salt, a tautomer, or a pharmaceutically acceptable salt of the tautomer thereof.

In a preferred embodiment, the metabolite is 6-((4′-(3-(2-carboxyethyl)-6-chloro-1H-indazol-5-yl)-[1,1′-biphenyl]-2-yl)oxy)-3,4,5-trihydroxytetrahydro-2H-pyran-2-carboxylic acid, a pharmaceutically acceptable salt, a tautomer, or a pharmaceutically acceptable salt of the tautomer thereof. In a preferred embodiment, the metabolite has the structure:

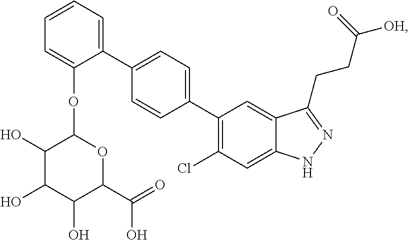 [SMILES: O=C(O)CCc1n[nH]c2cc(Cl)c(-c3ccc(-c4ccccc4OC4OC(C(=O)O)C(O)C(O)C4O)cc3)cc12]

a pharmaceutically acceptable salt, a tautomer, or a pharmaceutically acceptable salt of the tautomer thereof.

In a preferred embodiment, the metabolite is 3-(6-chloro-5-(2′-(sulfooxy)-[1,1′-biphenyl]-4-yl)-1H-indazol-3-yl)propanoic acid, a pharmaceutically acceptable salt, a tautomer, or a pharmaceutically acceptable salt of the tautomer thereof. In some embodiments, the metabolite is 3-(6-chloro-5-(2′-(sulfooxy)-[1,1′-biphenyl]-4-yl)-1H-indazol-3-yl)propanoic acid further comprising a hydroxyl group, or a pharmaceutically acceptable salt, a tautomer, or a pharmaceutically acceptable salt of the tautomer thereof. In some embodiments, the metabolite has the structure:

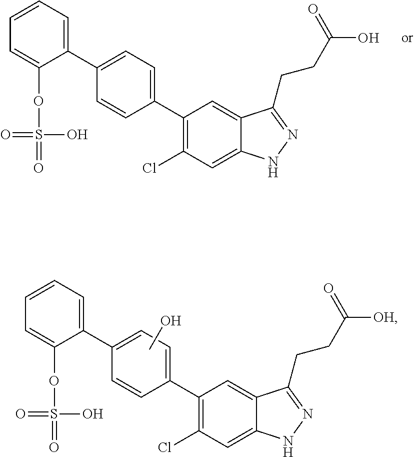

a pharmaceutically acceptable salt, a tautomer, or a pharmaceutically acceptable salt of the tautomer thereof.

### Pharmaceutical Compositions

In another embodiment, the disclosure comprises pharmaceutical compositions. For pharmaceutical composition purposes, the compound per se or pharmaceutically acceptable salt thereof will simply be referred to as the compounds of the disclosure.

The compositions of this disclosure may be in a variety of forms. These include, for example, liquid, semi-solid and solid dosage forms, such as liquid solutions (e.g., injectable and infusible solutions), dispersions or suspensions, tablets, capsules, pills, powders, liposomes and suppositories. The form depends on the intended mode of administration and therapeutic application.

Typical compositions are in the form of injectable or infusible solutions, such as compositions similar to those used for passive immunization of humans with antibodies in general. One mode of administration is parenteral (e.g., intravenous, subcutaneous, intraperitoneal, intramuscular). In another embodiment, the compound is administered by intravenous infusion or injection. In yet another embodiment, the compound is administered by intramuscular or subcutaneous injection.

Oral administration of a solid dosage form may be, for example, presented in discrete units, such as hard or soft capsules, pills, cachets, lozenges, or tablets, each containing a predetermined amount of at least one compound of the disclosure, or a pharmaceutically acceptable salt thereof. In another embodiment, the oral administration may be in a powder or granule form. In another embodiment, the oral dosage form is sub-lingual, such as, for example, a lozenge. In such solid dosage forms, the compounds of the disclosure are ordinarily combined with one or more adjuvants. Such capsules or tablets may comprise a controlled release formulation. In the case of capsules, tablets, and pills, the dosage forms also may comprise buffering agents or may be prepared with enteric coatings.

In another embodiment, oral administration may be in a liquid dosage form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as one or more of wetting, emulsifying, suspending, flavoring (e.g., sweetening), or perfuming agents.

In another embodiment, the disclosure comprises a parenteral dosage form. “Parenteral administration” includes, for example, subcutaneous injections, intravenous injections, intraperitoneally, intramuscular injections, intrasternal injections, and infusion. Injectable preparations (i.e., sterile injectable aqueous or oleaginous suspensions) may be formulated according to the known art using one or more of suitable dispersing, wetting agents, or suspending agents.

In another embodiment, the disclosure comprises a topical dosage form. “Topical administration” includes, for example, dermal and transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of this disclosure are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical excipients include alcohol, water, mineral oil, liquid petrolatum, white petrolatum, glycerin, polyethylene glycol and propylene glycol. Penetration enhancers may be incorporated—see, for example, FINNIN, B. C. and MORGAN, T. M., “Transdermal penetration enhancers: Applications, limitations, and potential,” Journal of Pharmaceutical Sciences, 1999, 88(10):955-958.

Formulations suitable for topical administration to the eye include, for example, eye drops wherein the compound of this disclosure is dissolved or suspended in a suitable excipient. A typical formulation suitable for ocular or aural administration may be in the form of drops of a micronized suspension or solution in isotonic, pH-adjusted, sterile saline. Other formulations suitable for ocular and aural administration include ointments, biodegradable (i.e., absorbable gel sponges, collagen) and non-biodegradable (i.e., silicone) implants, wafers, lenses and particulate or vesicular systems, such as niosomes or liposomes. A polymer such as crossed linked polyacrylic acid, polyvinyl alcohol, hyaluronic acid, a cellulosic polymer, for example, hydroxypropylmethylcellulose, hydroxyethylcellulose, or methylcellulose, or a heteropolysaccharide polymer, for example, gelan gum, may be incorporated together with a preservative, such as benzalkonium chloride. Such formulations may also be delivered by iontophoresis.

For intranasal administration, the compounds of the disclosure are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the disclosure comprises a rectal dosage form. Such rectal dosage form may be in the form of, for example, a suppository. Cocoa butter is a traditional suppository base, but various alternatives may be used as appropriate.

Other excipients and modes of administration known in the pharmaceutical art may also be used. Pharmaceutical compositions of the disclosure may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, ALLEN, L. V. and ANSEL, H. C. Ansel's Pharmaceutical Dosage Forms and Drug Delivery Systems. 10^{th} Ed. Philadelphia, Lippincott Williams & Wilkins, 2014; ADEJARE, A. Remington: The Science and Practice of Pharmacy. 23^{rd} Ed. Philadelphia, Lippincott Williams & Wilkins, 2000; ROWE, R. C., et al., Handbook of Pharmaceutical Excipients. 5^{th} Ed. Chicago, Pharmaceutical Press, 2006; STAHL, P. H. and WERMUTH, C. G., Pharmaceutical Salts: Properties, Selection, and Use. 2^{nd} Revised Ed. New York, Wiley-VCH, 2011; and BRITTAIN, H. G. Polymorphism in Pharmaceutical Solids. 2^{nd} Ed. CRC Press, 2009.

Acceptable excipients are nontoxic to subjects at the dosages and concentrations employed, and may comprise one or more of the following: 1) buffers such as phosphate, citrate, or other organic acids; 2) salts such as sodium chloride; 3) antioxidants such as ascorbic acid or methionine; 4) preservatives such as octadecyldimethylbenzyl ammonium chloride, hexamethonium chloride, benzalkonium chloride, benzethonium chloride, phenol, butyl or benzyl alcohol; 5) alkyl parabens such as methyl or propyl paraben, catechol, resorcinol, cyclohexanol, 3-pentanol, or m-cresol; 6) low molecular weight (less than about 10 residues) polypeptides; 7) proteins such as serum albumin, gelatin, or immunoglobulins; 8) hydrophilic polymers such as polyvinylpyrrolidone; 9) amino acids such as glycine, glutamine, asparagine, histidine, arginine, or lysine; 10) monosaccharides, disaccharides, or other carbohydrates including glucose, mannose, or dextrins; 11) chelating agents such as EDTA; 12) sugars such as sucrose, mannitol, trehalose or sorbitol; 13) salt-forming counter-ions such as sodium, metal complexes (e.g., Zn-protein complexes), or 14) non-ionic surfactants such as polysorbates (e.g., polysorbate 20 or polysorbate 80), poloxamers or polyethylene glycol (PEG).

For oral administration, the compositions may be provided in the form of tablets or capsules containing 0.01, 0.05, 0.1, 0.5, 1.0, 2.5, 5.0, 10.0, 15.0, 25.0, 50.0, 75.0, 100, 125, 150, 175, 200, 250 or 500 milligrams of the active ingredient for the symptomatic adjustment of the dosage to the patient. A medicament typically contains from about 0.01 mg to about 500 mg of the active ingredient, or in another embodiment, from about 1 mg to about 100 mg of active ingredient. Dosing regimens may depend on the route of administration, dose scheduling, and use of flat-dose, body surface area or weight-based dosing. For example, for weight-based dosing, intravenously doses may range from about 0.01 to about 10 mg/kg/minute during a constant rate infusion.

Liposome containing compounds of the disclosure may be prepared by methods known in the art (See, for example, CHANG, H. I. and YEH, M. K., “Clinical development of liposome-based drugs: formulation, characterization, and therapeutic efficacy,” International Journal of Nanomedicine, 2012, 7:49-60). Particularly useful liposomes may be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

Compounds of the disclosure may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in ADEJARE, A. Remington: The Science and Practice of Pharmacy. 23^{rd} Ed. Philadelphia, Lippincott Williams & Wilkins, 2000.

Sustained-release preparations may be used. Suitable examples of sustained-release preparations include semi-permeable matrices of solid hydrophobic polymers containing a compound of the disclosure, or a pharmaceutically acceptable salt thereof, which matrices are in the form of shaped articles, e.g., films, or microcapsules. Examples of sustained-release matrices include polyesters, hydrogels (for example, poly(2-hydroxyethyl-methacrylate), or ‘poly(vinylalcohol)), polylactides, copolymers of L-glutamic acid and 7 ethyl-L-glutamate, non-degradable ethylene-vinyl acetate, degradable lactic acid-glycolic acid copolymers such as those used in leuprolide acetate for depot suspension (injectable microspheres composed of lactic acid-glycolic acid copolymer and leuprolide acetate), sucrose acetate isobutyrate, and poly-D-(−)-3-hydroxybutyric acid.

The formulations to be used for intravenous administration must be sterile. This is readily accomplished by, for example, filtration through sterile filtration membranes. Compounds of the disclosure are generally placed into a container having a sterile access port, for example, an intravenous solution bag or vial having a stopper pierceable by a hypodermic injection needle.

Suitable emulsions may be prepared using commercially available fat emulsions, such as a lipid emulsions comprising soybean oil, a fat emulsion for intravenous administration (e.g., comprising safflower oil, soybean oil, egg phosphatides and glycerin in water), emulsions containing soya bean oil and medium-chain triglycerides, and lipid emulsions of cottonseed oil. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion may comprise fat droplets between 0.1 and 1.0 μm, particularly 0.1 and 0.5 μm, and have a pH in the range of 5.5 to 8.0.

For example, the emulsion compositions may be those prepared by mixing a compound of the disclosure, or a pharmaceutically acceptable salt thereof with a lipid emulsions comprising soybean oil or the components thereof (soybean oil, egg phospholipids, glycerol and water).

Compositions for inhalation or insufflation include solutions and suspensions in pharmaceutically acceptable aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

A drug product intermediate (DPI) is a partly processed material that must undergo further processing steps before it becomes bulk drug product. Compounds of the disclosure may be formulated into drug product intermediate DPI containing the active ingredient in a higher free energy form than the crystalline form. One reason to use a DPI is to improve oral absorption characteristics due to low solubility, slow dissolution, improved mass transport through the mucus layer adjacent to the epithelial cells, and in some cases, limitations due to biological barriers such as metabolism and transporters. Other reasons may include improved solid state stability and downstream manufacturability. In one embodiment, the drug product intermediate contains a compound of the disclosure, or a pharmaceutically acceptable salt thereof isolated and stabilized in the amorphous state (for example, amorphous solid dispersions (ASDs)). There are many techniques known in the art to manufacture ASD's that produce material suitable for integration into a bulk drug product, for example, spray dried dispersions (SDD's), melt extrudates (often referred to as HME's), co-precipitates, amorphous drug nanoparticles, and nano-adsorbates. In one embodiment amorphous solid dispersions comprise a compound of the disclosure, or a pharmaceutically acceptable salt thereof and a polymer excipient. Other excipients as well as concentrations of said excipients and the compound of the disclosure, or a pharmaceutically acceptable salt thereof are well known in the art and are described in standard textbooks. See, for example, SHAH, N., et al., Amorphous Solid Dispersions: Theory and Practice. New York, Springer, 2014.

### Extended Release Formulations

In one embodiment, an extended-release formulation of the disclosure comprises a compound disclosed herein, or a pharmaceutically acceptable salt thereof. The compositions of the disclosure comprise an amount of a compound disclosed herein, or a pharmaceutically acceptable salt thereof, that allows for content uniformity and efficacious dosing for the treatment of a condition disclosed herein. In some embodiments, an extended-release formulation of the disclosure comprises from about 5% to about 50%, about 5% to about 15%, about 15% to about 25%, about 25% to about 35%, about 35% to about 45%, about 25% to about 50%, about 10% to about 30%, or about 30% to about 50% w/w of a compound of the disclosure, or a pharmaceutically acceptable salt thereof. In some embodiments, an extended-release formulation of the disclosure comprises from about 15% to about 25% w/w of a compound of the disclosure, or a pharmaceutically acceptable salt thereof.

In some embodiments, an extended-release formulation of the disclosure comprises about 5%, about 10%, about 15%, about 20%, about 25%, about 30%, about 35%, about 40%, about 45%, or about 50% w/w of a compound of the disclosure, or a pharmaceutically acceptable salt thereof. In some embodiments, an extended-release formulation of the disclosure comprises about 5% w/w of a compound of the disclosure, or a pharmaceutically acceptable salt thereof. In some embodiments, an extended-release formulation of the disclosure comprises about 10% w/w of a compound of the disclosure, or a pharmaceutically acceptable salt thereof. In some embodiments, an extended-release formulation of the disclosure comprises about 15% w/w of a compound of the disclosure, or a pharmaceutically acceptable salt thereof. In some embodiments, an extended-release formulation of the disclosure comprises about 20% w/w of a compound of the disclosure, or a pharmaceutically acceptable salt thereof. In some embodiments, an extended-release formulation of the disclosure comprises about 25% w/w of a compound of the disclosure, or a pharmaceutically acceptable salt thereof. In some embodiments, an extended-release formulation of the disclosure comprises about 30% w/w of a compound of the disclosure, or a pharmaceutically acceptable salt thereof. In some embodiments, an extended-release formulation of the disclosure comprises about 35% w/w of a compound of the disclosure, or a pharmaceutically acceptable salt thereof. In some embodiments, an extended-release formulation of the disclosure comprises about 40% w/w of a compound of the disclosure, or a pharmaceutically acceptable salt thereof.

A composition of the disclosure comprises a compound disclosed herein, or a pharmaceutically acceptable salt thereof, at a particle size. In some embodiments, a compound of the disclosure, or a pharmaceutically acceptable salt thereof, is present in a composition with a D10 value of from about 0.5 μm to about 20 μm, from about 0.5 μm to about 5 μm, from about 5 μm to about 10 μm, from about 10 μm to about 15 μm, or from about 15 μm to about 20 μm. In some embodiments, a compound of the disclosure, or a pharmaceutically acceptable salt thereof, is present in a composition with a D10 value of from about 0.5 μm to about 5 μm. In some embodiments, a compound of the disclosure, or a pharmaceutically acceptable salt thereof, is present in a composition with a D10 value of from about 5 μm to about 10 μm.

In some embodiments, a compound of the disclosure, or a pharmaceutically acceptable salt thereof, is present in a composition with a D50 value of from about 1.5 μm to about 40 μm, from about 1.5 μm to about 10 μm, from about 10 μm to about 20 μm, from about 20 μm to about 30 μm, or from about 30 μm to about 40 μm. In some embodiments, a compound of the disclosure, or a pharmaceutically acceptable salt thereof, is present in a composition with a D50 value of from about 1.5 μm to about 40 μm. In some embodiments, a compound of the disclosure, or a pharmaceutically acceptable salt thereof, is present in a composition with a D50 value of from about 10 μm to about 20 μm. In some embodiments, a compound of the disclosure, or a pharmaceutically acceptable salt thereof, is present in a composition with a D50 value of from about 20 μm to about 30 μm. In some embodiments, a compound of the disclosure, or a pharmaceutically acceptable salt thereof, is present in a composition with a D50 value that is no more than about 10 μm. In some embodiments, a compound of the disclosure, or a pharmaceutically acceptable salt thereof, is present in a composition with a D50 value that is no more than about 20 μm. In some embodiments, a compound of the disclosure, or a pharmaceutically acceptable salt thereof, is present in a composition with a D50 value that is no more than about 25 μm. In some embodiments, a compound of the disclosure, or a pharmaceutically acceptable salt thereof, is present in a composition with a D50 value that is no more than about 30 μm.

In some embodiments, a compound of the disclosure, or a pharmaceutically acceptable salt thereof, is present in a composition with a D90 value of from about 3.5 μm to about 120 μm, from about 3.5 μm to about 20 μm, from about 20 μm to about 40 μm, from about 40 μm to about 60 μm, from about 60 μm to about 80 μm, from about 80 μm to about 100 μm, or from about 100 μm to about 120 μm. In some embodiments, a compound of the disclosure, or a pharmaceutically acceptable salt thereof, is present in a composition with a D90 value of from about 3.5 μm to about 120 μm. In some embodiments, a compound of the disclosure, or a pharmaceutically acceptable salt thereof, is present in a composition with a D90 value of from about 40 μm to about 60 μm. In some embodiments, a compound of the disclosure, or a pharmaceutically acceptable salt thereof, is present in a composition with a D90 value of from about 60 μm to about 80 μm. In some embodiments, a compound of the disclosure, or a pharmaceutically acceptable salt thereof, is present in a composition with a D90 value that is no more than about 60 μm. In some embodiments, a compound of the disclosure, or a pharmaceutically acceptable salt thereof, is present in a composition with a D90 value that is no more than about 75 μm. In some embodiments, a compound of the disclosure, or a pharmaceutically acceptable salt thereof, is present in a composition with a D90 value that is no more than about 80 μm.

In some embodiments, an extended release composition of the disclosure comprises a microsphere matrix component. In some embodiments, a microsphere matrix component can comprise a component selected from the group consisting of: stearyl alcohol, stearic acid, polyglyceryl-4-stearate, polyglyceryl-6-palmate, glyceryl behenate, behenic acid, stearic acid, hydrogenated cottonseed oil, carnauba wax, polyethylene glycol, and polyethylene oxide. In some embodiments, a microsphere matrix component comprises stearyl alcohol. In some embodiments, a microsphere matrix component comprises polyglyceryl-4-stearate. In some embodiments, a microsphere matrix component comprises polyglyceryl-6-palmate.

An extended release composition of the disclosure can comprise a microsphere matrix component in an amount of from about 25% to about 90% w/w, from about 25% to about 30% w/w, from about 30% to about 35% w/w, from about 35% to about 40% w/w, from about 40% to about 45% w/w, from about 45% to about 50% w/w, from about 50% to about 55% w/w, from about 55% to about 60% w/w, from about 60% to about 65% w/w, from about 65% to about 70% w/w, from about 70% to about 75% w/w, from about 75% to about 80% w/w, from about 80% to about 85% w/w, or from about 85% to about 90% w/w. In some embodiments, an extended release composition of the disclosure can comprise a microsphere matrix component in an amount of from about 20% to about 40% w/w. In some embodiments, an extended release composition of the disclosure can comprise a microsphere matrix component in an amount of from about 40% to about 60% w/w. In some embodiments, an extended release composition of the disclosure can comprise a microsphere matrix component in an amount of from about 60% to about 80% w/w. In some embodiments, an extended release composition of the disclosure can comprise a microsphere matrix component in an amount of from about 50% to about 60% w/w. In some embodiments, an extended release composition of the disclosure can comprise a microsphere matrix component in an amount of from about 60% to about 70% w/w. In some embodiments, an extended release composition of the disclosure can comprise a microsphere matrix component in an amount of from about 70% to about 80% w/w.

In some embodiments, an extended release composition of the disclosure can comprise a microsphere matrix component in an amount of about 20%, about 25%, about 30%, about 35%, about 40%, about 45%, about 50%, about 55%, about 60%, about 65%, about 70%, about 75%, about 80%, about 85%, or about 90% w/w. In some embodiments, an extended release composition of the disclosure can comprise a microsphere matrix component in an amount of about 50% w/w. In some embodiments, an extended release composition of the disclosure can comprise a microsphere matrix component in an amount of about 55% w/w. In some embodiments, an extended release composition of the disclosure can comprise a microsphere matrix component in an amount of about 60% w/w. In some embodiments, an extended release composition of the disclosure can comprise a microsphere matrix component in an amount of about 65% w/w. In some embodiments, an extended release composition of the disclosure can comprise a microsphere matrix component in an amount of about 68% w/w. In some embodiments, an extended release composition of the disclosure can comprise a microsphere matrix component in an amount of about 70% w/w.

In one embodiment, an extended release composition of the disclosure can comprise stearyl alcohol as a microsphere matrix component in an amount of about 60% to about 70% w/w. In one embodiment, an extended release composition of the disclosure can comprise stearyl alcohol as a microsphere matrix component in an amount of about 70% to about 80% w/w. In one embodiment, an extended release composition of the disclosure can comprise stearyl alcohol as a microsphere matrix component in an amount of about 65% w/w. In one embodiment, an extended release composition of the disclosure can comprise stearyl alcohol as a microsphere matrix component in an amount of about 68% w/w. In one embodiment, an extended release composition of the disclosure can comprise stearyl alcohol as a microsphere matrix component in an amount of about 70% w/w. In one embodiment, an extended release composition of the disclosure can comprise stearyl alcohol as a microsphere matrix component in an amount of about 75% w/w. In one embodiment, an extended release composition of the disclosure can comprise stearyl alcohol as a microsphere matrix component in an amount of about 76% w/w. In one embodiment, an extended release composition of the disclosure can comprise stearyl alcohol as a microsphere matrix component in an amount of about 80% w/w.

An extended release composition of the disclosure can comprise a microsphere release modifier. In some embodiments, the microsphere release modifier is a pore former, which enhances the porosity of a microsphere. In some embodiments, the microsphere release modifier is a disintegrant, which enhances the erodibility of a microsphere. In some embodiments, the pore former or disintegrant is selected from the group consisting of crospovidone, povidone, poloxamer 407 (β407), polyoxyethylene alkyl ethers, polyoxyethylene castor oil derivatives, polyoxyethylene sorbitan fatty acid esters, polyoxyethylene stearates, polyoxylglycerides, sodium starch glycolate type A (SSG-A), agar, croscarmellose sodium, alginic acid (and salts thereof, e.g. calcium alginate, sodium alginate), carboxymethylcellulose sodium, carboxymethylcellulose calcium, microcrystalline cellulose, chitosan, colloidal silicon dioxide, methylcellulose, polacrilin potassium, sodium chloride, potassium chloride, lactose, sucrose, fructose, dextrose, mannitol, cyclodextrins, citric acid, lactic acid, ammonium chloride, tromethamine (and salts thereof, e.g. tromethamine chloride), sodium citrate, sodium carbonate, sodium bicarbonate, potassium citrate, and potassium bicarbonate. In some embodiments, the pore former or disintegrant is crospovidone.

In some embodiments, the release modifier is insoluble polyvinylpyrrolidone. In some embodiments, the release modifier is crosslinked polyvinylpyrrolidone. In some embodiments, the release modifier is crospovidone. In some embodiments, the release modifier is crospovidone with a surface area of about 1.4 m²/g. In some embodiments, the release modifier is crospovidone with a surface area of about <1 m²/g. In some embodiments, the release modifier is crospovidone with a surface area of about 1.5 m²/g. In some embodiments, the release modifier is crospovidone with a surface area of about 3 m²/g. In some embodiments, the release modifier is crospovidone with a surface area of about >6 m²/g.

In some embodiments, the microsphere release modifier is a matrix excipient, which can modulate the release profile of a microsphere by making the combined microsphere matrix more or less hydrophilic. In some embodiments, the matrix excipient is selected from polyglyceryl-4-stearate, polyglycerol-3-behenate, and polyglyceryl-6-palmate.

An extended release composition of the disclosure can comprise a microsphere release modifier in an amount of from about 3% to about 30% w/w, from about 3% to about 5% w/w, from about 5% to about 10% w/w, from about 10% to about 15% w/w, from about 15% to about 20% w/w, from about 20% to about 25% w/w, or from about 25% to about 30% w/w. In some embodiments, an extended release composition of the disclosure can comprise a microsphere release modifier in an amount of from about 10% to about 15% w/w. In some embodiments, an extended release composition of the disclosure can comprise a microsphere release modifier in an amount of from about 15% to about 20% w/w.

An extended release composition of the disclosure can comprise a microsphere release modifier in an amount of about 3%, about 5%, about 10%, about 15%, about 20%, about 25%, or about 30% w/w. An extended release composition of the disclosure can comprise a microsphere release modifier in an amount of about 10% w/w. An extended release composition of the disclosure can comprise a microsphere release modifier in an amount of about 12% w/w. An extended release composition of the disclosure can comprise a microsphere release modifier in an amount of about 15% w/w. An extended release composition of the disclosure can comprise a microsphere release modifier in an amount of about 20% w/w.

In some embodiments, an extended release composition of the disclosure can comprise crospovidone as a microsphere release modifier in an amount of from about 3% to about 15% w/w. In some embodiments, an extended release composition of the disclosure can comprise crospovidone as a microsphere release modifier in an amount of about 5% w/w. In some embodiments, an extended release composition of the disclosure can comprise crospovidone as a microsphere release modifier in an amount of about 10% w/w. In some embodiments, an extended release composition of the disclosure can comprise crospovidone as a microsphere release modifier in an amount of about 12% w/w. In some embodiments, an extended release composition of the disclosure can comprise crospovidone as a microsphere release modifier in an amount of about 15% w/w.

An extended release composition of the disclosure can be in the form of a microsphere with a particle size that has a D10 of from about 50 μm to about 300 μm, from about 50 μm to about 100 μm, from about 100 μm to about 150 μm, from about 150 μm to about 200 μm, from about 200 μm to about 250 μm, or from about 250 μm to about 300 μm. In some embodiments, an extended release composition of the disclosure can be in the form of a microsphere with a particle size that has a D10 of from about 50 μm to about 300 μm. In some embodiments, an extended release composition of the disclosure can be in the form of a microsphere with a particle size that has a D10 of from about 100 μm to about 300 μm. In some embodiments, a compound of the disclosure, or a pharmaceutically acceptable salt thereof, is present in a composition with a D10 value that is no more than about 200 μm. In some embodiments, a compound of the disclosure, or a pharmaceutically acceptable salt thereof, is present in a composition with a D10 value that is no more than about 100 μm.

An extended release composition of the disclosure can be in the form of a microsphere with a particle size that has a D50 of from about 80 μm to about 400 μm, from about 80 μm to about 100 μm, from about 100 μm to about 150 μm, from about 150 μm to about 200 μm, from about 200 μm to about 250 μm, from about 250 μm to about 300 μm, from about 300 μm to about 350 μm, or from about 350 μm to about 400 μm. In some embodiments, an extended release composition of the disclosure can be in the form of a microsphere with a particle size that has a D50 of from about 100 μm to about 350 μm. In some embodiments, an extended release composition of the disclosure can be in the form of a microsphere with a particle size that has a D50 of from about 175 μm to about 225 μm. In some embodiments, an extended release composition of the disclosure can be in the form of a microsphere with a particle size that has a D50 of from about 200 μm to about 300 μm. In some embodiments, a compound of the disclosure, or a pharmaceutically acceptable salt thereof, is present in a composition with a D50 value that is no more than about 350 μm. In some embodiments, a compound of the disclosure, or a pharmaceutically acceptable salt thereof, is present in a composition with a D50 value that is no more than about 200 μm.

An extended release composition of the disclosure can be in the form of a microsphere with a particle size that has a D90 of from about 120 μm to about 750 μm, from about 120 μm to about 150 μm, from about 150 μm to about 200 μm, from about 200 μm to about 250 μm, from about 250 μm to about 300 μm, from about 300 μm to about 350 μm, from about 350 μm to about 400 μm, from about 400 μm to about 450 μm, from about 450 μm to about 500 μm, from about 500 μm to about 550 μm, from about 550 μm to about 600 μm, from about 600 μm to about 650 μm, from about 650 μm to about 700 μm, or from about 650 μm to about 700 μm. In some embodiments, an extended release composition of the disclosure can be in the form of a microsphere with a particle size that has a D90 of from about 150 μm to about 550 μm. In some embodiments, an extended release composition of the disclosure can be in the form of a microsphere with a particle size that has a D90 of from about 200 μm to about 300 μm. In some embodiments, a compound of the disclosure, or a pharmaceutically acceptable salt thereof, is present in a composition with a D90 value that is no more than about 550 μm. In some embodiments, a compound of the disclosure, or a pharmaceutically acceptable salt thereof, is present in a composition with a D10 value that is no more than about 250 μm.

An extended release composition of the disclosure can have a T80 value of from about 5 hrs to about 50 hrs, from about 5 hrs to about 10 hrs, from about 10 hrs to about 15 hrs, from about 15 hrs to about 20 hrs, from about 20 hrs to about 25 hrs, from about 25 hrs to about 30 hrs, from about 30 hrs to about 35 hrs, from about 35 hrs to about 40 hrs, from about 40 hrs to about 45 hrs, from about 45 hrs to about 50 hrs, from about 10 hrs to about 20 hrs, or from about 10 hrs to about 30 hrs. In some embodiments, an extended release composition of the disclosure can have a T80 value of from about 10 hrs to about 20 hrs. In some embodiments, an extended release composition of the disclosure can have a T80 value of from about 10 hrs to about 15 hrs.

An extended release composition of the disclosure can have a T80 value of at least about 2 hrs, at least about 5 hrs, at least about 10 hrs, at least about 15 hrs, at least about 20 hrs, at least about 25 hrs, at least about 30 hrs, at least about 35 hrs, at least about 40 hrs, at least about 45 hrs, or at least about 50 hrs. In some embodiments, an extended release composition of the disclosure can have a T80 value of at least about 5 hrs. In some embodiments, an extended release composition of the disclosure can have a T80 value of at least about 10 hrs. In some embodiments, an extended release composition of the disclosure can have a T80 value of at least about 15 hrs. In some embodiments, an extended release composition of the disclosure can have a T80 value of at least about 20 hrs.

An extended release composition of the disclosure can be in the form of a microcapsule. In some embodiments, a microcapsule can comprise a coating. In some embodiments, the microcapsule can comprise a pH-functional coating. In some embodiments, the microcapsule can comprise a sustained release coating. In some embodiments, the microcapsule can comprise an osmotic coating. In some embodiments, a microcapsule can comprise a coating in an amount of from about 10% to about 50%, from about 10% to about 15%, from about 15% to about 20%, from about 20% to about 25%, from about 25% to about 30%, from about 30% to about 35%, from about 35% to about 40%, from about 40% to about 45%, or from about 45% to about 50% coat wt % of the overall microcapsule formulation. In some embodiments, a microcapsule can comprise a coating in an amount of from about 25% to about 30% coat wt % of the overall microcapsule formulation. In some embodiments, a microcapsule can comprise a coating in an amount of from about 30% to about 35% coat wt % of the overall microcapsule formulation. In some embodiments, a microcapsule can comprise a coating in an amount of from about 35% to about 40% coat wt % of the overall microcapsule formulation.

In some embodiments, the coating comprises methacrylic acid, ethyl acrylate, methyl methacrylate, ethyl cellulose, or cellulose acetate. In some embodiments, the coating is selected from the group consisting of Eudragit® L 100-55, Eudragit® L 30D-55, Eudragit® FS 30D, PlasACR^{y}L® HTP20, Eudragit® L100, Eudragit® L 12.5, Eudragit® S100, Eudragit® S12.5, Eudragit® FS100, Eudragit® FS 30 D, and PlasACR^{y}L® T20. In some embodiments, the coating comprises a methacrylic acid and ethyl acrylate copolymer dispersion (Eudragit® L 30 D-55). In some embodiments, the coating comprises a 7:3:1 methyl acrylate:methyl methacrylate, and methacrylic acid copolymer dispersion (Eudragit® FS30D). In one embodiment, the coating comprises Eudragit® L 30D-55 and Eudragit® FS 30D.

In some embodiments, a microcapsule can comprise a coating comprising Eudragit® L 30D-55 in an amount of from about 25% to about 30% coat wt % of the overall microcapsule formulation. In some embodiments, a microcapsule can comprise a coating comprising Eudragit® L 30D-55 in an amount of from about 30% to about 35% coat wt % of the overall microcapsule formulation. In some embodiments, a microcapsule can comprise a coating comprising Eudragit® L 30D-55 in an amount of from about 35% to about 35% coat wt % of the overall microcapsule formulation.

In some embodiments, a microcapsule can comprise a coating comprising Eudragit® L 30D-55 and Eudragit® FS 30D in a ratio of about 1:1, about 2:1, about 3:1, about 4:1, about 5:1, about 1:5, about 1:4, about 1:3, or about 1:2. In some embodiments, a microcapsule can comprise a coating comprising Eudragit® L 30D-55 and Eudragit® FS 30D in a ratio of about 2:1. In some embodiments, a microcapsule can comprise a coating comprising Eudragit® L 30D-55 and Eudragit® FS 30D in a ratio of about 3:1. In some embodiments, a microcapsule can comprise a coating comprising Eudragit® L 30D-55 and Eudragit® FS 30D in a ratio of about 4:1. In one embodiment, a microcapsule can comprise a coating comprising Eudragit® L 30D-55 and Eudragit® FS 30D in a ratio of about 3:1, wherein the coating is in an amount of from about 25% to about 30% coat wt % of the overall microcapsule formulation. In one embodiment, a microcapsule can comprise a coating comprising Eudragit® L 30D-55 and Eudragit® FS 30D in a ratio of about 3:1, wherein the coating is in an amount of from about 30% to about 35% coat wt % of the overall microcapsule formulation. In one embodiment, a microcapsule can comprise a coating comprising Eudragit® L 30D-55 and Eudragit® FS 30D in a ratio of about 3:1, wherein the coating is in an amount of from about 35% to about 40% coat wt % of the overall microcapsule formulation. In one embodiment, a microcapsule can comprise a coating comprising Eudragit® L 30D-55 and Eudragit® FS 30D in a ratio of about 3:1, wherein the coating is in an amount of about 30% coat wt % of the overall microcapsule formulation. In one embodiment, a microcapsule can comprise a coating comprising Eudragit® L 30D-55 and Eudragit® FS 30D in a ratio of about 3:1, wherein the coating is in an amount of about 35% coat wt % of the overall microcapsule formulation.

An extended release microcapsule formulation can comprise a coating that initiates release of the active pharmaceutical ingredient at a pH of from about 5 to about 7, from about 5 to about 5.5, from about 5.5 to about 6, from about 6 to about 6.5, or from about 6.5 to about 7. In some embodiments, an extended release microcapsule formulation can comprise a coating that initiates release of the active pharmaceutical ingredient at a pH of from about 5 to about 6. In some embodiments, an extended release microcapsule formulation can comprise a coating that initiates release of the active pharmaceutical ingredient at a pH of from about 6 to about 7.

In some embodiments, an extended release microcapsule formulation can comprise a coating that initiates release of the active pharmaceutical ingredient at about pH≥5.0. In some embodiments, an extended release microcapsule formulation can comprise a coating that initiates release of the active pharmaceutical ingredient at about pH≥5.5. In some embodiments, an extended release microcapsule formulation can comprise a coating that initiates release of the active pharmaceutical ingredient at about pH≥6.0. In some embodiments, an extended release microcapsule formulation can comprise a coating that initiates release of the active pharmaceutical ingredient at about pH≥6.5. In some embodiments, an extended release microcapsule formulation can comprise a coating that initiates release of the active pharmaceutical ingredient at about pH≥7.0.

In some embodiments, an extended release microcapsule formulation can comprise a coating that initiates release of the active pharmaceutical ingredient at a pH of about 5.5. In some embodiments, an extended release microcapsule formulation can comprise a coating that initiates release of the active pharmaceutical ingredient at a pH of about 6. In some embodiments, an extended release microcapsule formulation can comprise a coating that initiates release of the active pharmaceutical ingredient at a pH of about 6.5. In some embodiments, an extended release microcapsule formulation can comprise a coating that initiates release of the active pharmaceutical ingredient at a pH of about 7.

An extended release microcapsule formulation can have a particle size that has a D10 of from about 100 μm to about 200 μm, from about 100 μm to about 120 μm, from about 120 μm to about 140 μm, from about 140 μm to about 160 μm, from about 160 μm to about 180 μm, or from about 180 μm to about 200 μm. In some embodiments, an extended release microcapsule formulation can have a particle size that has a D10 of from about 100 μm to about 200 μm. In some embodiments, an extended release microcapsule formulation can have a particle size that has a D10 of from about 100 μm to about 120 μm. In some embodiments, an extended release microcapsule formulation can have a particle size that has a D10 of from about 120 μm to about 140 μm. In some embodiments, an extended release microcapsule formulation can have a particle size that has a D10 of from about 140 μm to about 160 μm.

An extended release microcapsule formulation can have a particle size that has a D50 of from about 200 μm to about 300 μm, from about 200 μm to about 220 μm, from about 220 μm to about 240 μm, from about 240 μm to about 260 μm, from about 260 μm to about 280 μm, or from about 280 μm to about 300 μm. In some embodiments, an extended release microcapsule formulation can have a particle size that has a D50 of from about 200 μm to about 220 μm. In some embodiments, an extended release microcapsule formulation can have a particle size that has a D50 of from about 220 μm to about 240 μm. In some embodiments, an extended release microcapsule formulation can have a particle size that has a D50 of from about 240 μm to about 260 μm.

An extended release microcapsule formulation can have a particle size that has a D90 of from about 300 μm to about 400 μm, from about 300 μm to about 320 μm, from about 320 μm to about 340 μm, from about 340 μm to about 360 μm, from about 360 μm to about 380 μm, or from about 380 μm to about 400 μm. In some embodiments, an extended release microcapsule formulation can have a particle size that has a D90 of from about 300 μm to about 320 μm. In some embodiments, an extended release microcapsule formulation can have a particle size that has a D90 of from about 320 μm to about 340 μm. In some embodiments, an extended release microcapsule formulation can have a particle size that has a D90 of from about 340 μm to about 360 μm.

In one embodiment, an extended release formulation of the disclosure is prepared as a tablet. In one embodiment, an extended release microcapsule formulation of the disclosure is prepared as a tablet. In one embodiment, an extended release formulation of the disclosure is filled into a capsule. In one embodiment, an extended release microcapsule formulation of the disclosure is filled into a capsule. In one embodiment, an extended release formulation of the disclosure is filled into a sachet or stick pack. In one embodiment, an extended release microcapsule formulation of the disclosure is filled into a sachet or stick pack.

### Method of Making Extended Release Microsphere Formulations

Disclosed herein are methods of making a microsphere formulation comprising a compound of the disclosure, or a pharmaceutically acceptable salt thereof. In one embodiment, a melt-spray-congeal process is used to make a microsphere formulation disclosed herein. In another embodiment, a twin-screw extruder with in-line rotary atomizer is used to make a microsphere formulation disclosed herein. In another embodiment, hand homogenization of a melted blend, followed by rotary atomizationis used to make a microsphere formulation disclosed herein.

In one embodiment, a melt-spray-congeal process is used to make a microsphere formulation disclosed herein. In some embodiments, the melt-spray-congeal process comprises the steps of:
1) blending and/or milling of solid excipients to prepare a milled blend;
2) transferring the milled blend to a gravimetric powder feeder;
3) delivering the milled blend by the gravimetric powder feeder to a twin-screw extruder;
4) heating and mixing the milled blend to prepare a melt suspension;
5) conveying the melt suspension to a spinning disk;
6) atomizing the melt suspension on the spinning disk to generate microsphere particles;
7) collecting the microsphere particles and passing the microsphere particles through a high throughout screener to prepare screened microsphere particles; and
8) annealing the screened microsphere particles.

In one embodiment, hand homogenization of a melted blend, followed by rotary atomization is used to make a microsphere formulation disclosed herein. In some embodiments, the hand homogenization and rotary atomization process comprises heating the solid excipients and compound of the disclosure, or a pharmaceutically acceptable salt thereof, to melt the matrix component; using an overhead paddle mixer or homogenizer to suspend the solids and mix the matrix; and transferring the resulting melt suspension to a spinning disk using a syringe pump.

### Method of Making Extended Release Microcapsules

Disclosed herein are methods of making extended release microcapsules comprising microsphere particles, wherein the microsphere particles comprise a compound of the disclosure, or a pharmaceutically acceptable salt thereof. In some embodiments, a coating suspension is prepared.

### Administration and Dosing

A compound of the invention is administered in an amount effective to treat a condition as described herein. The compounds of the invention may be administered as compound per se, or alternatively, as a pharmaceutically acceptable salt, tautomer, or pharmaceutically acceptable salt of the tautomer. For administration and dosing purposes, the compound per se, pharmaceutically acceptable salt, tautomer, or pharmaceutically acceptable salt of the tautomer thereof will simply be referred to as the compounds of the invention.

The compounds of the invention are administered by any suitable route in the form of a pharmaceutical composition adapted to such a route, and in a dose effective for the treatment intended. The compounds of the invention may be administered orally, rectally, vaginally, parenterally, topically, intranasally, or by inhalation.

In a preferred embodiment, the compounds of the invention may be administered orally. Oral administration may involve swallowing, so that the compound enters the gastrointestinal tract, or buccal or sublingual administration may be employed by which the compound enters the bloodstream directly from the mouth.

In another embodiment, the compounds of the invention may also be administered parenterally, for example directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors, and infusion techniques.

In another embodiment, the compounds of the invention may also be administered topically to the skin or mucosa, that is, dermally or transdermally. In another embodiment, the compounds of the invention may also be administered intranasally or by inhalation. In another embodiment, the compounds of the invention may be administered rectally or vaginally. In another embodiment, the compounds of the invention may also be administered directly to the eye or ear.

The dosage regimen for the compounds of the invention or compositions containing said compounds is based on a variety of factors, including the type, age, weight, sex and medical condition of the patient; the severity of the condition; the route of administration; and the activity of the particular compound employed. Thus, the dosage regimen may vary widely. In one embodiment, the total daily dose of a compound of the invention is typically from about 0.01 to about 100 mg/kg (i.e., mg compound of the invention per kg body weight) for the treatment of the indicated conditions discussed herein. In another embodiment, total daily dose of the compound of the invention is from about 0.1 to about 50 mg/kg, and in another embodiment, from about 0.5 to about 30 mg/kg. It is not uncommon that the administration of the compounds of the invention will be repeated a plurality of times in a day (typically no greater than 4 times). Multiple doses per day typically may be used to increase the total daily dose, if desired. In some embodiments, the compound, pharmaceutically acceptable salt, tautomer, or pharmaceutically acceptable salt of the tautomer thereof is administered once a day, twice a day, or three times a day. In a preferred embodiment, the compound, pharmaceutically acceptable salt, tautomer, or pharmaceutically acceptable salt of the tautomer thereof is administered once a day. In a preferred embodiment, the compound, pharmaceutically acceptable salt, tautomer, or pharmaceutically acceptable salt of the tautomer thereof is administered twice a day. In a preferred embodiment, the compound, pharmaceutically acceptable salt, tautomer, or pharmaceutically acceptable salt of the tautomer thereof is administered three times a day.

In one embodiment, a compound of the disclosure, a pharmaceutically acceptable salt thereof, a tautomer thereof, or a pharmaceutically acceptable salt of the tautomer thereof, or a pharmaceutical composition comprising the compound or a pharmaceutically acceptable salt thereof, a tautomer thereof, or a pharmaceutically acceptable salt of the tautomer thereof, may be administered orally in the form of a tablet or a capsule. The dosage of the compound, pharmaceutically acceptable salt, tautomer, or pharmaceutically acceptable salt of the tautomer thereof may be adjusted based on the patient's response and symptoms. In some embodiments, the compound or pharmaceutical composition may provide the compound in an amount of from about 0.01 mg to about 150 mg, from about 150 mg to about 250 mg, from about 250 mg to about 500 mg, from about 500 mg to about 750 mg, from about 750 mg to about 1000 mg, from about 1250 mg to about 1500 mg, from about 1500 mg to about 1750 mg, from about 1750 mg to about 2000 mg, from about 2000 mg to about 2250 mg, from about 2250 mg to about 2500 mg, from about 2500 mg to about 2750 mg, from about 2750 mg to about 3000 mg, from about 3000 mg to about 3250 mg, from about 3250 mg to about 3500 mg, from about 3500 mg to about 3750 mg, from about 3750 mg to about 4000 mg, from about 4000 mg to about 4250 mg, from about 4250 mg to about 4500 mg, from about 4500 mg to about 4750 mg, or from about 4750 mg to about 5000 mg. In a preferred embodiment, the compound, pharmaceutically acceptable salt, tautomer, or pharmaceutically acceptable salt of the tautomer thereof may be provided in an amount of from about 1 mg to about 2500 mg. In a preferred embodiment, the compound, pharmaceutically acceptable salt, tautomer, or pharmaceutically acceptable salt of the tautomer thereof may be provided in an amount of from about 1 mg to about 100 mg. In a preferred embodiment, the compound, pharmaceutically acceptable salt, tautomer, or pharmaceutically acceptable salt of the tautomer thereof may be provided in an amount of from about 1 mg to about 50 mg. In a preferred embodiment, the compound, pharmaceutically acceptable salt, tautomer, or pharmaceutically acceptable salt of the tautomer thereof may be provided in an amount of from about 1 mg to about 25 mg. In some embodiments, the compound, pharmaceutically acceptable salt, tautomer, or pharmaceutically acceptable salt of the tautomer thereof may be provided in an amount of from about 150 mg to about 2500 mg. In some embodiments, the compound, pharmaceutically acceptable salt, tautomer, or pharmaceutically acceptable salt of the tautomer thereof may be provided in an amount of from about 150 mg to about 500 mg. In a preferred embodiment, the compound, pharmaceutically acceptable salt, tautomer, or pharmaceutically acceptable salt of the tautomer thereof may be provided in an amount of from about 100 mg to about 1000 mg. In some embodiments, the compound, pharmaceutically acceptable salt, tautomer, or pharmaceutically acceptable salt of the tautomer thereof may be provided in an amount of from about 500 mg to about 1500 mg. In some embodiments, the compound, pharmaceutically acceptable salt, tautomer, or pharmaceutically acceptable salt of the tautomer thereof may be provided in an amount of from about 1500 mg to about 2500 mg. In some embodiments, the compound, pharmaceutically acceptable salt, tautomer, or pharmaceutically acceptable salt of the tautomer thereof may be provided in an amount of from about 2500 mg to about 5000 mg.

A compound, pharmaceutically acceptable salt, tautomer, or pharmaceutically acceptable salt of the tautomer thereof disclosed herein may be provided in an amount of about 0.01 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 50 mg, about 100 mg, about 150 mg, about 200 mg, about 250 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, about 3000 mg, about 3200 mg, about 3400 mg, about 3600 mg, about 3800 mg, about 4000 mg, about 4200 mg, about 4400 mg, about 4600 mg, about 4800 mg, or about 5000 mg. In a preferred embodiment, the compound, pharmaceutically acceptable salt, tautomer, or pharmaceutically acceptable salt of the tautomer thereof disclosed herein may be provided in an amount of about 5 mg. In a preferred embodiment, the compound, pharmaceutically acceptable salt, tautomer, or pharmaceutically acceptable salt of the tautomer thereof disclosed herein may be provided in an amount of about 10 mg. In a preferred embodiment, the compound, pharmaceutically acceptable salt, tautomer, or pharmaceutically acceptable salt of the tautomer thereof disclosed herein may be provided in an amount of about 15 mg. In a preferred embodiment, the compound, pharmaceutically acceptable salt, tautomer, or pharmaceutically acceptable salt of the tautomer thereof disclosed herein may be provided in an amount of about 25 mg. In a preferred embodiment, the compound, pharmaceutically acceptable salt, tautomer, or pharmaceutically acceptable salt of the tautomer thereof disclosed herein may be provided in an amount of about 50 mg. In a preferred embodiment, the compound, pharmaceutically acceptable salt, tautomer, or pharmaceutically acceptable salt of the tautomer thereof disclosed herein may be provided in an amount of about 75 mg. In a preferred embodiment, the compound, pharmaceutically acceptable salt, tautomer, or pharmaceutically acceptable salt of the tautomer thereof disclosed herein may be provided in an amount of about 100 mg. In a preferred embodiment, the compound, pharmaceutically acceptable salt, tautomer, or pharmaceutically acceptable salt of the tautomer thereof disclosed herein may be provided in an amount of about 250 mg. In a preferred embodiment, the compound, pharmaceutically acceptable salt, tautomer, or pharmaceutically acceptable salt of the tautomer thereof disclosed herein may be provided in an amount of about 500 mg. In a preferred embodiment, the compound, pharmaceutically acceptable salt, tautomer, or pharmaceutically acceptable salt of the tautomer thereof disclosed herein may be provided in an amount of about 1000 mg.

### Therapeutic Methods and Uses

The compounds of the disclosure may activate AMPK and may be useful in the treatment of a condition associated with AMPK. In a preferred embodiment, the compounds of the disclosure may be a pan-AMPK activator and may be useful in the treatment of a condition associated with AMPK. In some embodiments, the condition or disorder is a metabolic disorder, an inflammatory disorder, an autoimmune disorder, a disorder of gastrointestinal barrier dysfunction, a functional gastrointestinal disorder, an eating disorder, a nutritional disorder, an allergy, or a central nervous system (CNS) disorder. In a preferred embodiment, the AMPK-activating compounds of the disclosure may be administered to a subject in need thereof to treat a metabolic disorder. In a preferred embodiments, the AMPK-activating compounds of the disclosure may be administered to a subject in need thereof to treat an inflammatory or autoimmune disorder. In a preferred embodiments, the AMPK-activating compounds of the disclosure may be administered to a subject in need thereof to treat a disorder of gastrointestinal barrier dysfunction or a functional gastrointestinal disorder.

In some embodiments, the AMPK-activating compounds disclosed herein may treat a metabolic disorder or a complication resulting from a metabolic condition selected from the group consisting of type 2 diabetes, gestational diabetes, insulin resistance, hyperglycemia, hypercholesterolemia, hypertriglyceridemia (elevated levels of triglyceride-rich-lipoproteins), obesity, abdominal obesity, vascular restenosis, hyperinsulinemia, glucose intolerance, atherosclerosis, Metabolic Syndrome, hypertension, high hepatic glucose output, high blood glucose concentrations, nonalcoholic steatohepatitis (NASH), dyslipidemia, mixed dyslipidemia, diabetic dyslipidemia, protection against ischemia and reperfusion damage, a lipid disorder, elevated levels of plasma triglycerides, elevated levels of free fatty acids, elevated levels of cholesterol, high levels of low density lipoprotein (LDL), low levels of high density lipoprotein (HDL), chronic kidney disease, diabetic nephropathy, diabetic retinopathy, diabetic neuropathy, cardiovascular disease, hypoxia, cancer, non-alcoholic fatty liver disease (NAFLD), glucocorticoid-induced apoptosis, loss of skeletal muscle mass, sarcopenia, high circulating free fatty acids (FFAs), heart attack, cardiomyopathy, heart failure, and atherosclerosis. In a preferred embodiment, the AMPK activating compounds of the disclosure may treat a metabolic disorder selected from the group consisting of type 2 diabetes, gestational diabetes, hyperglycemia, metabolic syndrome, obesity, hypercholesterolemia, or hypertension.

In some embodiments, the AMPK activating compounds disclosed herein may treat an inflammatory disorder or an autoimmune disorder selected from the group consisting of inflammatory bowel disease, ulcerative colitis, Crohn's disease, checkpoint inhibitor-induced colitis, psoriasis, celiac disease, graft-versus-host disease (GVHD), radiation-induced enteritis, chemotherapy-induced enteritis, and necrotizing enterocolitis. In some embodiments, the AMPK-activating compounds disclosed herein may treat gastrointestinal injury resulting from toxic insults such as radiation or chemotherapy. In a preferred embodiment, the AMPK-activating compounds of the disclosure may treat an inflammatory disorder or an autoimmune disorder selected from the group consisting of inflammatory bowel disease, colitis, ulcerative colitis, and Crohn's disease. In a preferred embodiment, the AMPK-activating compounds of the disclosure may treat inflammatory bowel disease. In a preferred embodiment, the AMPK-activating compounds of the disclosure may treat colitis. In a preferred embodiment, the AMPK-activating compounds of the disclosure may treat ulcerative colitis. In a preferred embodiment, the AMPK-activating compounds of the disclosure may treat Crohn's disease.

In some embodiments, the AMPK-activating compounds disclosed herein may treat a disorder of gastrointestinal barrier dysfunction, such as environmental enteric dysfunction or spontaneous bacterial peritonitis. In some embodiments, the AMPK-activating compounds disclosed herein may treat ischemic colitis or sclerosing cholangitis.

In some embodiments, the AMPK-activating compounds disclosed herein may treat a functional gastrointestinal disorder selected from the group consisting of irritable bowel syndrome, functional dyspepsia, functional abdominal bloating, functional abdominal distension, functional diarrhea, functional constipation, gastroparesis, disorders related to microbiome dysbiosis, and opioid-induced constipation. In a preferred embodiment, the AMPK-activating compounds of the disclosure may treat a functional gastrointestinal disorder selected from the group consisting of irritable bowel syndrome, functional diarrhea, celiac disease, and functional constipation.

In some embodiments, the AMPK-activating compounds disclosed herein may treat an eating disorder or a nutritional disorder selected from the group consisting of hyperphagia, cachexia, anorexia nervosa, short bowel syndrome, intestinal failure, and intestinal insufficiency.

In some embodiments, the AMPK-activating compounds of the disclosure may treat complications associated with an eating disorder or a nutritional disorder, such as left ventricular hypertrophy.

In some embodiments, the AMPK-activating compounds disclosed herein may treat an allergy, such as food allergies and celiac sprue. In some embodiments, the AMPK-activating compounds disclosed herein may treat nausea and vomiting.

In some embodiments, the AMPK-activating compounds disclosed herein may treat a central nervous system disorder selected from the group consisting of a mood disorder, anxiety, depression, an affective disorder, schizophrenia, malaise, cognition disorder, addiction, autism, epilepsy, a neurodegenerative disorder, Alzheimer's disease, Parkinson's disease, Lewy Body dementia, episodic cluster headaches, migraines, and pain.

In some embodiments, the AMPK-activating compounds of the disclosure can decrease fatty acid synthesis; increase fatty acid oxidation; increase ketogenesis; decrease cholesterol synthesis, lipogenesis, and/or triglyceride synthesis; decrease blood glucose levels and/or concentrations; improve glucose homeostasis; normalize glucose metabolism; decrease blood pressure; increase HDL levels; decrease LDL levels; decrease plasma triglyceride levels; decrease fatty acid levels; decrease hepatic glucose output; improve insulin action; decrease blood pressure; improve insulin sensitivity; suppress hepatic glucose output; inhibit de novo lipogenesis; simulate muscle glucose uptake; modulate insulin secretion by pancreatic p cells; decrease body weight; increase skeletal muscle mass; or prevent the loss of skeletal muscle mass. In a preferred embodiment, the AMPK-activating compounds disclosed herein can treat or reduce systemic infection or systemic inflammation from having a leaky gut barrier. In a preferred embodiment, the AMPK-activating compounds disclosed herein are pan-AMPK activators.

### Co-Administration

The compounds of the invention may be used alone, or in combination with one or more other therapeutic agents. The invention provides any of the uses, methods or compositions as defined herein wherein the compound of the invention, pharmaceutically acceptable salt, tautomer, or pharmaceutically acceptable salt of the tautomer thereof, is used in combination with one or more other therapeutic agent discussed herein.

The administration of two or more compounds “in combination” means that all of the compounds are administered closely enough in time to affect treatment of the subject. The two or more compounds may be administered simultaneously or sequentially, via the same or different routes of administration, on same or different administration schedules and with or without specific time limits depending on the treatment regimen. Additionally, simultaneous administration may be carried out by mixing the compounds prior to administration or by administering the compounds at the same point in time but as separate dosage forms at the same or different site of administration. Examples of “in combination” include, but are not limited to, “concurrent administration,” “co-administration,” “simultaneous administration,” “sequential administration” and “administered simultaneously”.

A compound of the invention and the one or more other therapeutic agents may be administered as a fixed or non-fixed combination of the active ingredients. The term “fixed combination” means a compound of the invention, pharmaceutically acceptable salt, tautomer, or pharmaceutically acceptable salt of the tautomer thereof, and the one or more therapeutic agents, are both administered to a subject simultaneously in a single composition or dosage. The term “non-fixed combination” means that a compound of the invention, pharmaceutically acceptable salt, tautomer, or pharmaceutically acceptable salt of the tautomer thereof, and the one or more therapeutic agents are formulated as separate compositions or dosages such that they may be administered to a subject in need thereof simultaneously or at different times with variable intervening time limits, wherein such administration provides effective levels of the two or more compounds in the body of the subject.

In one embodiment, the compounds of this invention are administered in combination with the specifically named agents including the pharmaceutically acceptable salts of the specifically named agents and the pharmaceutically acceptable solvates of said agents and salts.

The present invention also provides any of the uses, methods or compositions as defined above wherein the compound of Formula I, Ia, II, III, IVa-c, and V, pharmaceutically acceptable salt, tautomer, or pharmaceutically acceptable salt of the tautomer thereof, is used in combination with another pharmacologically active compound, particularly one of the functionally-defined classes or specific compounds listed below. These agents may be administered as part of the same or separate dosage forms, via the same or different routes of administration, and on the same or different administration schedules according to standard pharmaceutical practice known to one skilled in the art.

Suitable agents for use in combination therapy with a compound of Formula I, Ia, II, III, IVa-c, and V, pharmaceutically acceptable salt, tautomer, or pharmaceutically acceptable salt of the tautomer thereof, include: sulfasalazine, mesalazine, prednisone, azathioprine, infliximab, adalimumab, belimumab, becertolizumab, natalizumab, vedolizumab, hydrocortisone, budesonide, cyclosporin, tacrolimus, fexofenadine, 6-mercaptopurine, methotrexate, ursodeoxycholic acid, obeticholic acid, anti-histamines, rifampin, prednisone, methotrexate, azathioprine, cyclophosphamide, hydroxychloroquine, mofetil, sodium mycophenolate, tacrolimus, leflunomide, chloroquine and quinacrine, thalidomide, rituxan, NSAIDs, solumedrol, depomedrol, and dexamethasone.

Other suitable agents for use in combination therapy with a compound of Formula I, Ia, II, III, IVa-c, and V, pharmaceutically acceptable salt, tautomer, or pharmaceutically acceptable salt of the tautomer thereof, include: a 5-lipoxygenase activating protein (FLAP) antagonist; a leukotriene antagonist (LTRA) such as an antagonist of LTB₄, LTC₄, LTD₄, LTE₄, CysLT₁ or CysLT₂, e.g., montelukast or zafirlukast; a histamine receptor antagonist, such as a histamine type 1 receptor antagonist or a histamine type 2 receptor antagonist, e.g., loratidine, fexofenadine, desloratidine, levocetirizine, methapyrilene or cetirizine; an α1-adrenoceptor agonist or an α2-adrenoceptor agonist, e.g., phenylephrine, methoxamine, oxymetazoline or methylnorephrine; a muscarinic M3 receptor antagonist, e.g., tiotropium or ipratropium; a dual muscarinic M3 receptor antagononist/β2 agonist; a PDE inhibitor, such as a PDE3 inhibitor, a PDE4 inhibitor or a PDE5 inhibitor, e.g., theophylline, sildenafil, vardenafil, tadalafil, ibudilast, cilomilast or roflumilast; sodium cromoglycate or sodium nedocromil; a cyclooxygenase (COX) inhibitor, such as a non-selective inhibitor (e.g., aspirin or ibuprofen) or a selective inhibitor (e.g., celecoxib or valdecoxib); a glucocorticosteroid, e.g., fluticasone, mometasone, dexamethasone, prednisolone, budesonide, ciclesonide or beclamethasone; an anti-inflammatory monoclonal antibody, e.g., infliximab, adalimumab, tanezumab, ranibizumab, bevacizumab or mepolizumab; a β2 agonist, e.g., salmeterol, albuterol, salbutamol, fenoterol or formoterol, particularly a long-acting β2 agonist; an integrin antagonist, e.g., natalizumab; an adhesion molecule inhibitor, such as a VLA-4 antagonist; a kinin B₁ or B₂ receptor antagonist; an immunosuppressive agent, such as an inhibitor of the IgE pathway (e.g., omalizumab) or cyclosporine; a matrix metalloprotease (MMP) inhibitor, such as an inhibitor of MMP-9 or MMP-12; a tachykinin NK₁, NK₂ or NK₃ receptor antagonist; a protease inhibitor, such as an inhibitor of elastase, chymase or catheopsin G; an adenosine A₂ₐ receptor agonist; an adenosine A_{2b} receptor antagonist; a urokinase inhibitor; a dopamine receptor agonist (e.g., ropinirole), particularly a dopamine D2 receptor agonist (e.g., bromocriptine); a modulator of the NFκB pathway, such as an IKK inhibitor; a further modulator of a cytokine signaling pathway such as an inhibitor of syk kinase, p38 kinase, SPHK-1 kinase, Rho kinase, EGF-R or MK-2; a mucolytic, mucokinetic or anti-tussive agent; an antibiotic; an antiviral agent; a vaccine; a chemokine; an epithelial sodium channel (ENaC) blocker or Epithelial sodium channel (EnaC) inhibitor; a nucleotide receptor agonist, such as a β2Y2 agonist; a thromboxane inhibitor; niacin; a 5-lipoxygenase (5-LO) inhibitor, e.g., Zileuton; an adhesion factor, such as VLAM, ICAM or ELAM; a CRTH2 receptor (DP₂) antagonist; a prostaglandin D2 receptor (DP₁) antagonist; a hematopoietic prostaglandin D2 synthase (HPGDS) inhibitor; interferon-p; a soluble human TNF receptor, e.g., Etanercept; a HDAC inhibitor; a phosphoinositide 3-kinase gamma (PI3Kγ) inhibitor; a phosphoinositide 3-kinase delta (PI3Kδ) inhibitor; a CXCR-1 or a CXCR-2 receptor antagonist; an IRAK-4 inhibitor; and, a TLR-4 or TLR-9 inhibitor, including the pharmaceutically acceptable salts of the specifically named compounds. The agents may be administered with another active agent, wherein the second active agent may be administered either orally or topically.

These agents and compounds of the invention may be combined with pharmaceutically acceptable vehicles such as saline, Ringer's solution, dextrose solution, and the like. The particular dosage regimen, i.e., dose, timing and repetition, will depend on the particular individual and that individual's medical history.

### Kits

Another aspect of the invention provides kits comprising the compound of the invention or pharmaceutical compositions comprising the compound of the invention. A kit may include, in addition to the compound of the invention or pharmaceutical composition thereof, diagnostic or therapeutic agents. A kit may also include instructions for use in a diagnostic or therapeutic method. In some embodiments, the kit includes the compound or a pharmaceutical composition thereof and a diagnostic agent. In other embodiments, the kit includes the compound or a pharmaceutical composition thereof and one or more therapeutic agents, such as the therapeutic agents for co-administration described herein.

In yet another embodiment, the invention comprises kits that are suitable for use in performing the methods of treatment described herein. In one embodiment, the kit contains a first dosage form comprising one or more of the compounds of the invention in quantities sufficient to carry out the methods of the invention. In another embodiment, the kit comprises one or more compounds of the invention in quantities sufficient to carry out the methods of the invention and a container for the dosage and a container for the dosage.

### EXAMPLES

In order that this invention may be better understood, the following examples are set forth. These examples are for purposes of illustration only and are not to be construed as limiting the scope of the invention in any manner.

### Example 1: AMPK-Activating Compounds

The Examples in Table 1 were prepared using methods described in PCT/IB2024/053239.

TABLE 1
AMPK-activating compounds

| Ex# | Structure | IUPAC Name |
|---|---|---|
| 1 | 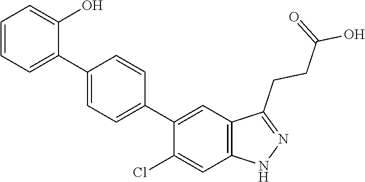 [SMILES: O=C(O)CCc1n[nH]c2cc(Cl)c(-c3ccc(-c4ccccc4O)cc3)cc12] | 3-(6-Chloro-5-(2′-hydroxy-[1,1′- biphenyl]-4-yl)-1H-indazol-3- yl)propanoic acid |
| 2 | 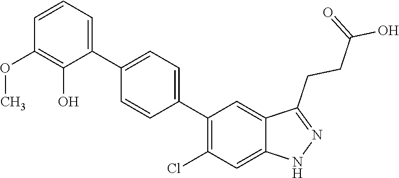 [SMILES: COc1cccc(-c2ccc(-c3cc4c(CCC(=O)O)n[nH]c4cc3Cl)cc2)c1O] | 3-(6-Chloro-5-(2′-hydroxy-3′- methoxy-[1,1′-biphenyl]-4-yl)- 1H-indazol-3-yl)propanoic acid |
| 3 | 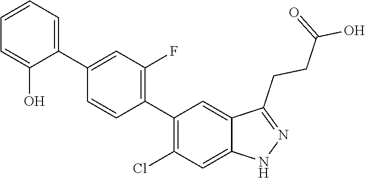 [SMILES: O=C(O)CCc1n[nH]c2cc(Cl)c(-c3ccc(-c4ccccc4O)cc3F)cc12] | 3-(6-Chloro-5-(3-fluoro-2′- hydroxy-[1,1′-biphenyl]-4-yl)- 1H-indazol-3-yl)propanoic acid |
| 4 | 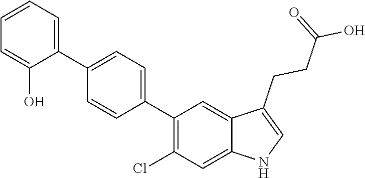 [SMILES: O=C(O)CCc1c[nH]c2cc(Cl)c(-c3ccc(-c4ccccc4O)cc3)cc12] | 3-(6-Chloro-5-(2′-hydroxy-[1,1′- biphenyl]-4-yl)-1H-indol-3- yl)propanoic acid |
| 5 | 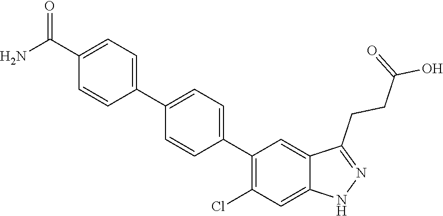 [SMILES: NC(=O)c1ccc(-c2ccc(-c3cc4c(CCC(=O)O)n[nH]c4cc3Cl)cc2)cc1] | 3-(5-(4′-Carbamoyl-[1,1′- biphenyl]-4-yl)-6-chloro-1H- indazol-3-yl)propanoic acid |
| 6 | 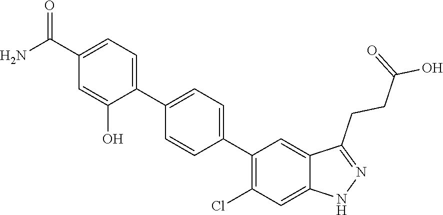 [SMILES: NC(=O)c1ccc(-c2ccc(-c3cc4c(CCC(=O)O)n[nH]c4cc3Cl)cc2)c(O)c1] | 3-(5-(4′-Carbamoyl-2′-hydroxy- [1,1′-biphenyl]-4-yl)-6-chloro- 1H-indazol-3-yl)propanoic acid |
| 7 | 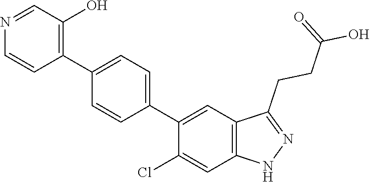 [SMILES: O=C(O)CCc1n[nH]c2cc(Cl)c(-c3ccc(-c4ccncc4O)cc3)cc12] | 3-(6-Chloro-5-(4-(3- hydroxypyridin-4-yl)phenyl)- 1H-indazol-3-yl)propanoic acid |
| 8 | 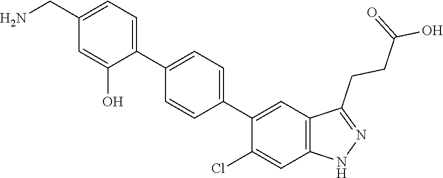 [SMILES: NCc1ccc(-c2ccc(-c3cc4c(CCC(=O)O)n[nH]c4cc3Cl)cc2)c(O)c1] | 3-(5-(4′-(Aminomethyl)-2′- hydroxy-[1,1′-biphenyl]-4-yl)-6- chloro-1H-indazol-3- yl)propanoic acid |
| 9 | 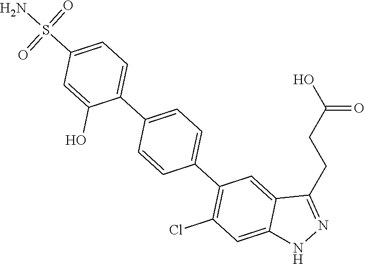 [SMILES: NS(=O)(=O)c1ccc(-c2ccc(-c3cc4c(CCC(=O)O)n[nH]c4cc3Cl)cc2)c(O)c1] | 3-(6-chloro-5-(2′-hydroxy-4′- sulfamoyl-[1,1′-biphenyl]-4-yl)- 1H-indazol-3-yl)propanoic acid |
| 10 | 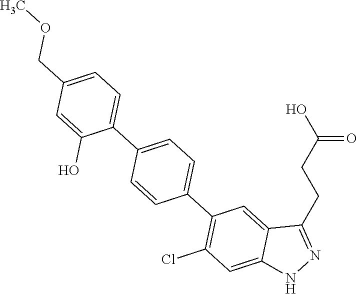 [SMILES: COCc1ccc(-c2ccc(-c3cc4c(CCC(=O)O)n[nH]c4cc3Cl)cc2)c(O)c1] | 3-(6-chloro-5-(2′-hydroxy-4′- (methoxymethyl)-[1,1′- biphenyl]-4-yl)-1H-indazol-3- yl)propanoic acid |
| 11 | 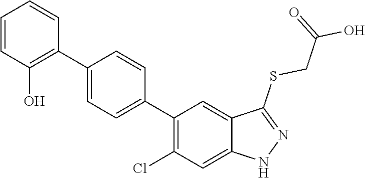 [SMILES: O=C(O)CSc1n[nH]c2cc(Cl)c(-c3ccc(-c4ccccc4O)cc3)cc12] | 2-((6-chloro-5-(2′-hydroxy-[1,1′- biphenyl]-4-yl)-1H-indazol-3- yl)thio)acetic acid |
| 12 | 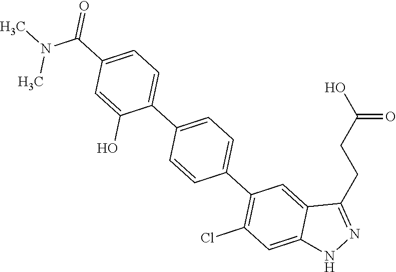 [SMILES: CN(C)C(=O)c1ccc(-c2ccc(-c3cc4c(CCC(=O)O)n[nH]c4cc3Cl)cc2)c(O)c1] | 3-(6-chloro-5-(4′- (dimethylcarbamoyl)-2′- hydroxy-[1,1′-biphenyl]-4-yl)- 1H-indazol-3-yl)propanoic acid |
| 13 | 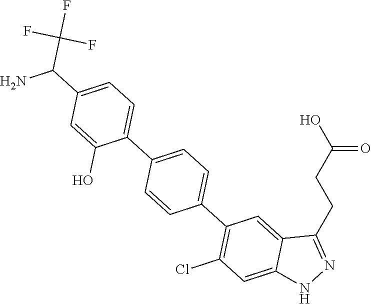 [SMILES: NC(c1ccc(-c2ccc(-c3cc4c(CCC(=O)O)n[nH]c4cc3Cl)cc2)c(O)c1)C(F)(F)F] | 3-(5-(4′-(1-amino-2,2,2- trifluoroethyl)-2′-hydroxy-[1,1′- biphenyl]-4-yl)-6-chloro-1H- indazol-3-yl)propanoic acid |
| 14 | 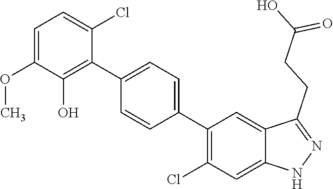 [SMILES: COc1ccc(Cl)c(-c2ccc(-c3cc4c(CCC(=O)O)n[nH]c4cc3Cl)cc2)c1O] | 3-(6-chloro-5-(6′-chloro-2′- hydroxy-3′-methoxy-[1,1′- biphenyl]-4-yl)-1H-indazol-3- yl)propanoic acid |
| 15 | 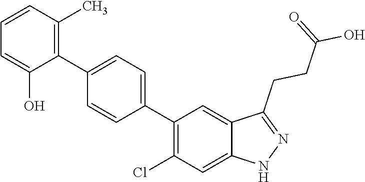 [SMILES: Cc1cccc(O)c1-c1ccc(-c2cc3c(CCC(=O)O)n[nH]c3cc2Cl)cc1] | 3-(6-chloro-5-(2′-hydroxy-6′- methyl-[1,1′-biphenyl]-4-yl)-1H- indazol-3-yl)propanoic acid |
| 16 | 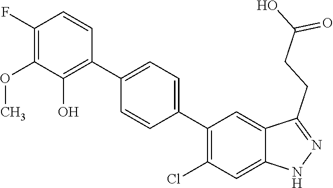 [SMILES: COc1c(F)ccc(-c2ccc(-c3cc4c(CCC(=O)O)n[nH]c4cc3Cl)cc2)c1O] | 3-(6-chloro-5-(4′-fluoro-2′- hydroxy-3′-methoxy-[1,1′- biphenyl]-4-yl)-1H-indazol-3- yl)propanoic acid |
| 17 | 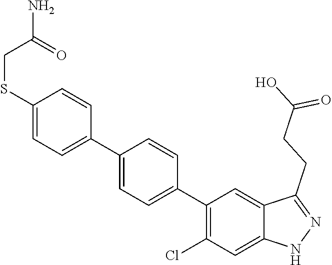 [SMILES: NC(=O)CSc1ccc(-c2ccc(-c3cc4c(CCC(=O)O)n[nH]c4cc3Cl)cc2)cc1] | 3-(5-(4′-((2-amino-2- oxoethyl)thio)-[1,1′-biphenyl]-4- yl)-6-chloro-1H-indazol-3- yl)propanoic acid |
| 18 | 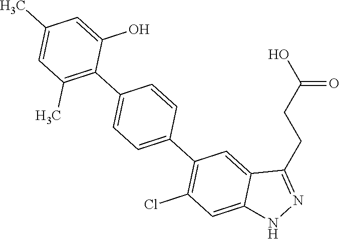 [SMILES: Cc1cc(C)c(-c2ccc(-c3cc4c(CCC(=O)O)n[nH]c4cc3Cl)cc2)c(O)c1] | 3-(6-chloro-5-(2′-hydroxy-4′,6′- dimethyl-[1,1′-biphenyl]-4-yl)- 1H-indazol-3-yl)propanoic acid |
| 19 | 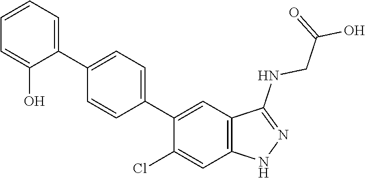 [SMILES: O=C(O)CNc1n[nH]c2cc(Cl)c(-c3ccc(-c4ccccc4O)cc3)cc12] | (6-Chloro-5-(2′-hydroxy-[1,1′- biphenyl]-4-yl)-1H-indazol-3- yl)glycine |
| 20 | 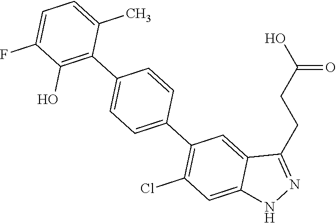 [SMILES: Cc1ccc(F)c(O)c1-c1ccc(-c2cc3c(CCC(=O)O)n[nH]c3cc2Cl)cc1] | 3-(6-chloro-5-(3′-fluoro-2′- hydroxy-6′-methyl-[1,1′- biphenyl]-4-yl)-1H-indazol-3- yl)propanoic acid |
| 21 | 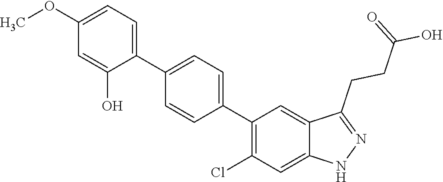 [SMILES: COc1ccc(-c2ccc(-c3cc4c(CCC(=O)O)n[nH]c4cc3Cl)cc2)c(O)c1] | 3-(6-chloro-5-(2′-hydroxy-4′- methoxy-[1,1′-biphenyl]-4-yl)- 1H-indazol-3-yl)propanoic acid |
| 22 | 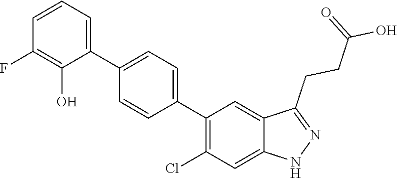 [SMILES: O=C(O)CCc1n[nH]c2cc(Cl)c(-c3ccc(-c4cccc(F)c4O)cc3)cc12] | 3-(6-chloro-5-(3′-fluoro-2′- hydroxy-[1,1′-biphenyl]-4-yl)- 1H-indazol-3-yl)propanoic acid |
| 23 | 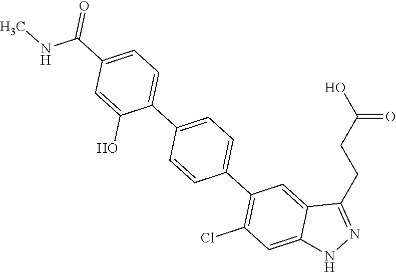 [SMILES: CNC(=O)c1ccc(-c2ccc(-c3cc4c(CCC(=O)O)n[nH]c4cc3Cl)cc2)c(O)c1] | 3-(6-chloro-5-(2′-hydroxy-4′- (methylcarbamoyl)-[1,1′- biphenyl]-4-yl)-1H-indazol-3- yl)propanoic acid |
| 24 | 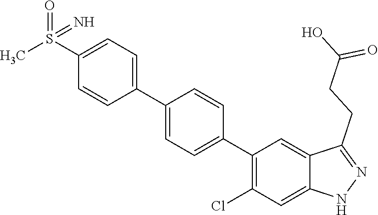 [SMILES: CS(=N)(=O)c1ccc(-c2ccc(-c3cc4c(CCC(=O)O)n[nH]c4cc3Cl)cc2)cc1] | 3-(6-chloro-5-(4′-(S- methylsulfonimidoyl)-[1,1′- biphenyl]-4-yl)-1H-indazol-3- yl)propanoic acid |
| 25 | 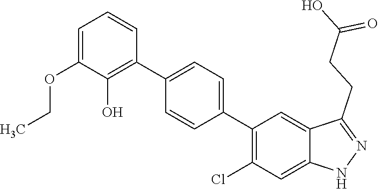 [SMILES: CCOc1cccc(-c2ccc(-c3cc4c(CCC(=O)O)n[nH]c4cc3Cl)cc2)c1O] | 3-(6-chloro-5-(3′-ethoxy-2′- hydroxy-[1,1′-biphenyl]-4-yl)- 1H-indazol-3-yl)propanoic acid |
| 26 | 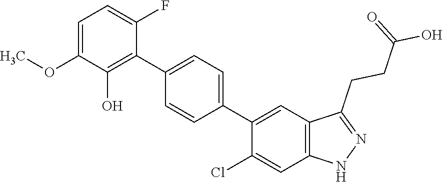 [SMILES: COc1ccc(F)c(-c2ccc(-c3cc4c(CCC(=O)O)n[nH]c4cc3Cl)cc2)c1O] | 3-(6-chloro-5-(6′-fluoro-2′- hydroxy-3′-methoxy-[1,1′- biphenyl]-4-yl)-1H-indazol-3- yl)propanoic acid |
| 27 | 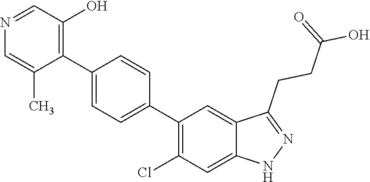 [SMILES: Cc1cncc(O)c1-c1ccc(-c2cc3c(CCC(=O)O)n[nH]c3cc2Cl)cc1] | 3-(6-chloro-5-(4-(3-hydroxy-5- methylpyridin-4-yl)phenyl)-1H- indazol-3-yl)propanoic acid |
| 28 | 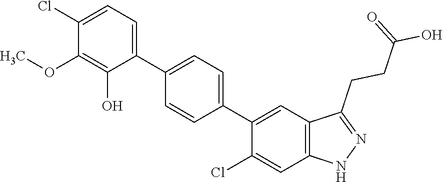 [SMILES: COc1c(Cl)ccc(-c2ccc(-c3cc4c(CCC(=O)O)n[nH]c4cc3Cl)cc2)c1O] | 3-(6-chloro-5-(4′-chloro-2′- hydroxy-3′-methoxy-[1,1′- biphenyl]-4-yl)-1H-indazol-3- yl)propanoic acid |
| 29 | 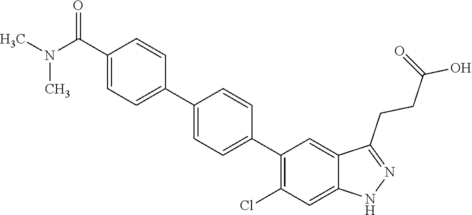 [SMILES: CN(C)C(=O)c1ccc(-c2ccc(-c3cc4c(CCC(=O)O)n[nH]c4cc3Cl)cc2)cc1] | 3-(6-chloro-5-(4′- (dimethylcarbamoyl)-[1,1′- biphenyl]-4-yl)-1H-indazol-3- yl)propanoic acid |
| 30 | 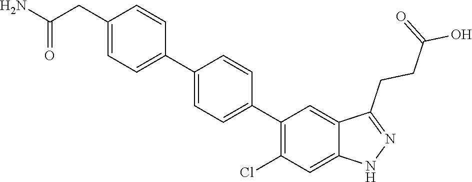 [SMILES: NC(=O)Cc1ccc(-c2ccc(-c3cc4c(CCC(=O)O)n[nH]c4cc3Cl)cc2)cc1] | 3-(5-(4′-(2-amino-2-oxoethyl)- [1,1′-biphenyl]-4-yl)-6-chloro- 1H-indazol-3-yl)propanoic acid |
| 31 | 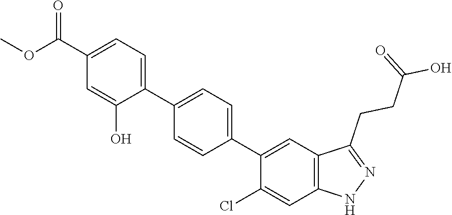 [SMILES: COC(=O)c1ccc(-c2ccc(-c3cc4c(CCC(=O)O)n[nH]c4cc3Cl)cc2)c(O)c1] | 3-(6-Chloro-5-(2′-hydroxy-4′- (methoxycarbonyl)-[1,1′- biphenyl]-4-yl)-1H-indazol-3- yl)propanoic acid |
| 32 | 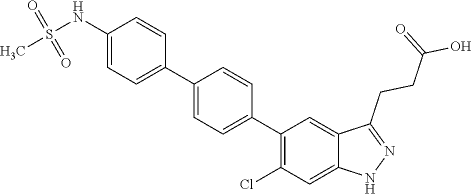 [SMILES: CS(=O)(=O)Nc1ccc(-c2ccc(-c3cc4c(CCC(=O)O)n[nH]c4cc3Cl)cc2)cc1] | 3-(6-chloro-5-(4′- (methylsulfonamido)-[1,1′- biphenyl]-4-yl)-1H-indazol-3- yl)propanoic acid |
| 33 | 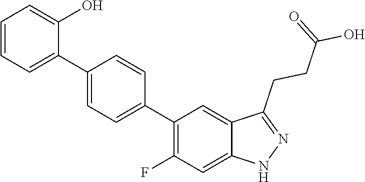 [SMILES: O=C(O)CCc1n[nH]c2cc(F)c(-c3ccc(-c4ccccc4O)cc3)cc12] | 3-(6-fluoro-5-(2′-hydroxy-[1,1′- biphenyl]-4-yl)-1H-indazol-3- yl)propanoic acid |
| 34 | 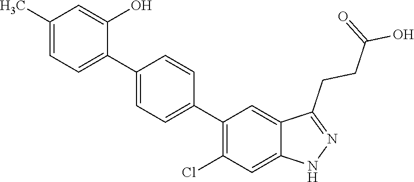 [SMILES: Cc1ccc(-c2ccc(-c3cc4c(CCC(=O)O)n[nH]c4cc3Cl)cc2)c(O)c1] | 3-(6-chloro-5-(2′-hydroxy-4′- methyl-[1,1′-biphenyl]-4-yl)-1H- indazol-3-yl)propanoic acid |
| 35 | 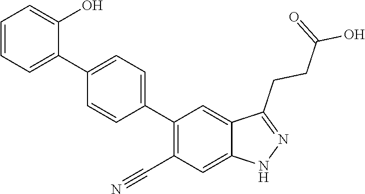 [SMILES: N#Cc1cc2[nH]nc(CCC(=O)O)c2cc1-c1ccc(-c2ccccc2O)cc1] | 3-(6-cyano-5-(2′-hydroxy-[1,1′- biphenyl]-4-yl)-1H-indazol-3- yl)propanoic acid |
| 36 | 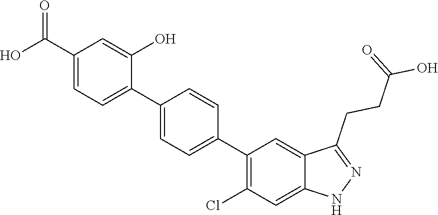 [SMILES: O=C(O)CCc1n[nH]c2cc(Cl)c(-c3ccc(-c4ccc(C(=O)O)cc4O)cc3)cc12] | 4′-(3-(2-carboxyethyl)-6-chloro- 1H-indazol-5-yl)-2-hydroxy- [1,1′-biphenyl]-4-carboxylic acid |
| 37 | 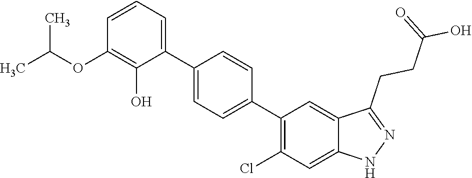 [SMILES: CC(C)Oc1cccc(-c2ccc(-c3cc4c(CCC(=O)O)n[nH]c4cc3Cl)cc2)c1O] | 3-(6-chloro-5-(2′-hydroxy-3′- isopropoxy-[1,1′-biphenyl]-4- yl)-1H-indazol-3-yl)propanoic acid |
| 38 | 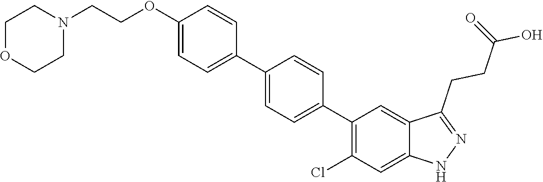 [SMILES: O=C(O)CCc1n[nH]c2cc(Cl)c(-c3ccc(-c4ccc(OCCN5CCOCC5)cc4)cc3)cc12] | 3-(6-chloro-5-(4′-(2- morpholinoethoxy)-[1,1′- biphenyl]-4-yl)-1H-indazol-3- yl)propanoic acid |
| 39 | 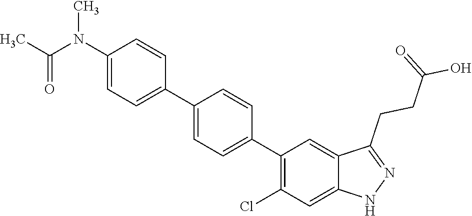 [SMILES: CC(=O)N(C)c1ccc(-c2ccc(-c3cc4c(CCC(=O)O)n[nH]c4cc3Cl)cc2)cc1] | 3-(6-chloro-5-(4′-(N- methylacetamido)-[1,1′- biphenyl]-4-yl)-1H-indazol-3- yl)propanoic acid |
| 40 | 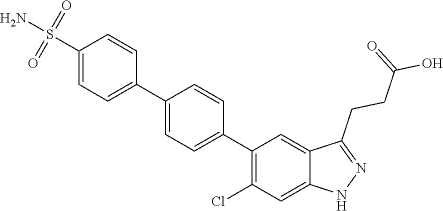 [SMILES: NS(=O)(=O)c1ccc(-c2ccc(-c3cc4c(CCC(=O)O)n[nH]c4cc3Cl)cc2)cc1] | 3-(6-chloro-5-(4′-sulfamoyl- [1,1′-biphenyl]-4-yl)-1H- indazol-3-yl)propanoic acid |
| 41 | 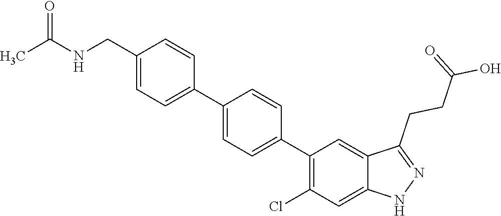 [SMILES: CC(=O)NCc1ccc(-c2ccc(-c3cc4c(CCC(=O)O)n[nH]c4cc3Cl)cc2)cc1] | 3-(5-(4′-(acetamidomethyl)- [1,1′-biphenyl]-4-yl)-6-chloro- 1H-indazol-3-yl)propanoic acid |
| 42 | 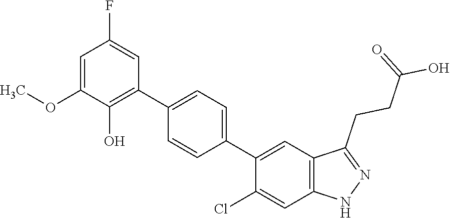 [SMILES: COc1cc(F)cc(-c2ccc(-c3cc4c(CCC(=O)O)n[nH]c4cc3Cl)cc2)c1O] | 3-(6-chloro-5-(5′-fluoro-2′- hydroxy-3′-methoxy-[1,1′- biphenyl]-4-yl)-1H-indazol-3- yl)propanoic acid |
| 43 | 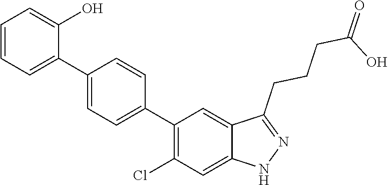 [SMILES: O=C(O)CCCc1n[nH]c2cc(Cl)c(-c3ccc(-c4ccccc4O)cc3)cc12] | 4-(6-chloro-5-(2′-hydroxy-[1,1′- biphenyl]-4-yl)-1H-indazol-3- yl)butanoic acid |
| 44 | 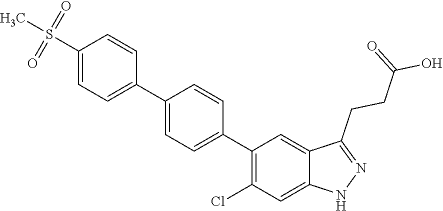 [SMILES: CS(=O)(=O)c1ccc(-c2ccc(-c3cc4c(CCC(=O)O)n[nH]c4cc3Cl)cc2)cc1] | 3-(6-chloro-5-(4′- (methylsulfonyl)-[1,1′- biphenyl]-4-yl)-1H-indazol-3- yl)propanoic acid |
| 45 | 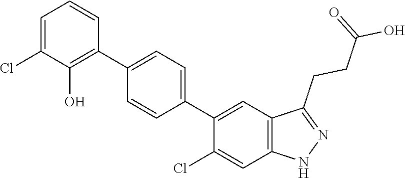 [SMILES: O=C(O)CCc1n[nH]c2cc(Cl)c(-c3ccc(-c4cccc(Cl)c4O)cc3)cc12] | 3-(6-chloro-5-(3′-chloro-2′- hydroxy-[1,1′-biphenyl]-4-yl)- 1H-indazol-3-yl)propanoic acid |
| 46 | 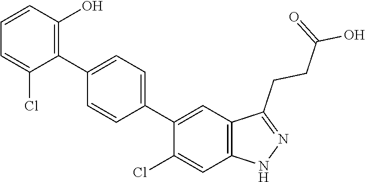 [SMILES: O=C(O)CCc1n[nH]c2cc(Cl)c(-c3ccc(-c4c(O)cccc4Cl)cc3)cc12] | 3-(6-chloro-5-(2′-chloro-6′- hydroxy-[1,1′-biphenyl]-4-yl)- 1H-indazol-3-yl)propanoic acid |
| 47 | 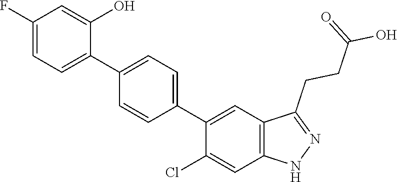 [SMILES: O=C(O)CCc1n[nH]c2cc(Cl)c(-c3ccc(-c4ccc(F)cc4O)cc3)cc12] | 3-(6-chloro-5-(4′-fluoro-2′- hydroxy-[1,1′-biphenyl]-4-yl)- 1H-indazol-3-yl)propanoic acid |
| 48 | 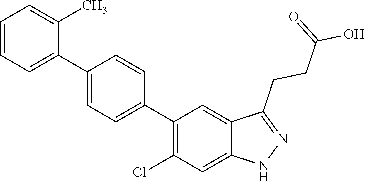 [SMILES: Cc1ccccc1-c1ccc(-c2cc3c(CCC(=O)O)n[nH]c3cc2Cl)cc1] | 3-(6-chloro-5-(2′-methyl-[1,1′- biphenyl]-4-yl)-1H-indazol-3- yl)propanoic acid |
| 49 | 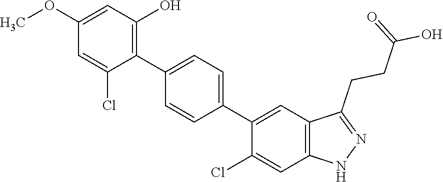 [SMILES: COc1cc(O)c(-c2ccc(-c3cc4c(CCC(=O)O)n[nH]c4cc3Cl)cc2)c(Cl)c1] | 3-(6-chloro-5-(2′-chloro-6′- hydroxy-4′-methoxy-[1,1′- biphenyl]-4-yl)-1H-indazol-3- yl)propanoic acid |
| 50 | 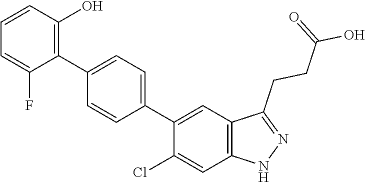 [SMILES: O=C(O)CCc1n[nH]c2cc(Cl)c(-c3ccc(-c4c(O)cccc4F)cc3)cc12] | 3-(6-chloro-5-(2′-fluoro-6′- hydroxy-[1,1′-biphenyl]-4-yl)- 1H-indazol-3-yl)propanoic acid |
| 51 | 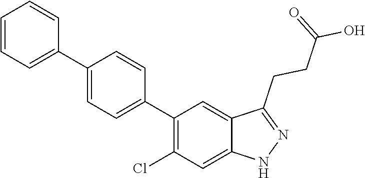 [SMILES: O=C(O)CCc1n[nH]c2cc(Cl)c(-c3ccc(-c4ccccc4)cc3)cc12] | 3-(5-([1,1′-biphenyl]-4-yl)-6- chloro-1H-indazol-3- yl)propanoic acid |
| 52 | 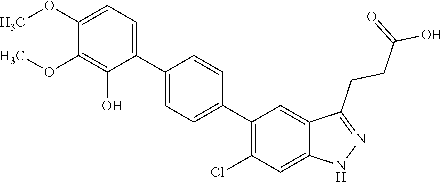 [SMILES: COc1ccc(-c2ccc(-c3cc4c(CCC(=O)O)n[nH]c4cc3Cl)cc2)c(O)c1OC] | 3-(6-chloro-5-(2′-hydroxy-3′,4′- dimethoxy-[1,1′-biphenyl]-4-yl)- 1H-indazol-3-yl)propanoic acid |
| 53 | 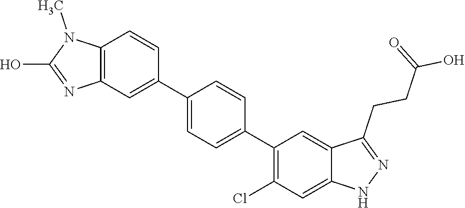 [SMILES: Cn1c(O)nc2cc(-c3ccc(-c4cc5c(CCC(=O)O)n[nH]c5cc4Cl)cc3)ccc21] | 3-(6-chloro-5-(4-(2-hydroxy-1- methyl-1H-benzo[d]imidazol-5- yl)phenyl)-1H-indazol-3- yl)propanoic acid |
| 54 | 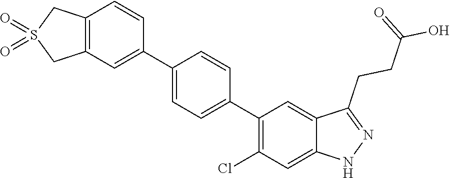 [SMILES: O=C(O)CCc1n[nH]c2cc(Cl)c(-c3ccc(-c4ccc5c(c4)CS(=O)(=O)C5)cc3)cc12] | 3-(6-chloro-5-(4-(2,2-dioxido- 1,3-dihydrobenzo[c]thiophen-5- yl)phenyl)-1H-indazol-3- yl)propanoic acid |
| 55 | 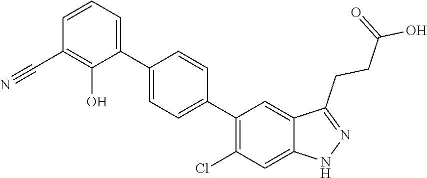 [SMILES: N#Cc1cccc(-c2ccc(-c3cc4c(CCC(=O)O)n[nH]c4cc3Cl)cc2)c1O] | 3-(6-chloro-5-(3′-cyano-2′- hydroxy-[1,1′-biphenyl]-4-yl)- 1H-indazol-3-yl)propanoic acid |
| 56 | 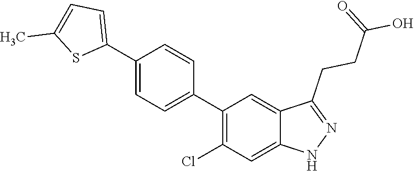 [SMILES: Cc1ccc(-c2ccc(-c3cc4c(CCC(=O)O)n[nH]c4cc3Cl)cc2)s1] | 3-(6-chloro-5-(4-(5- methylthiophen-2-yl)phenyl)- 1H-indazol-3-yl)propanoic acid |
| 57 | 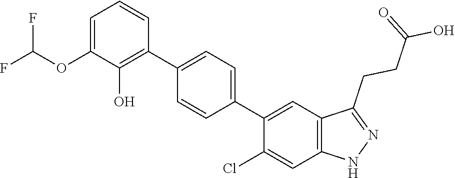 [SMILES: O=C(O)CCc1n[nH]c2cc(Cl)c(-c3ccc(-c4cccc(OC(F)F)c4O)cc3)cc12] | 3-(6-chloro-5-(3′- (difluoromethoxy)-2′-hydroxy- [1,1′-biphenyl]-4-yl)-1H- indazol-3-yl)propanoic acid |
| 58 | 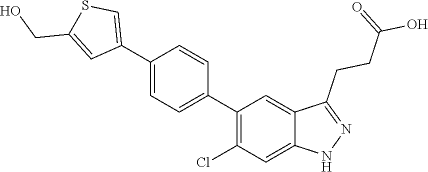 [SMILES: O=C(O)CCc1n[nH]c2cc(Cl)c(-c3ccc(-c4csc(CO)c4)cc3)cc12] | 3-(6-chloro-5-(4-(5- (hydroxymethyl)thiophen-3- yl)phenyl)-1H-indazol-3- yl)propanoic acid |
| 59 | 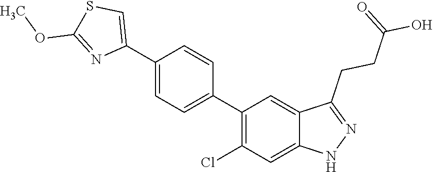 [SMILES: COc1nc(-c2ccc(-c3cc4c(CCC(=O)O)n[nH]c4cc3Cl)cc2)cs1] | 3-(6-chloro-5-(4-(2- methoxythiazol-4-yl)phenyl)- 1H-indazol-3-yl)propanoic acid |
| 60 | 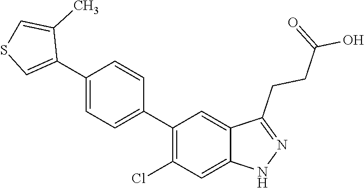 [SMILES: Cc1cscc1-c1ccc(-c2cc3c(CCC(=O)O)n[nH]c3cc2Cl)cc1] | 3-(6-chloro-5-(4-(4- methylthiophen-3-yl)phenyl)- 1H-indazol-3-yl)propanoic acid |
| 61 | 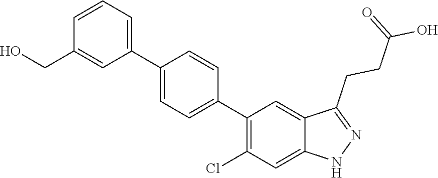 [SMILES: O=C(O)CCc1n[nH]c2cc(Cl)c(-c3ccc(-c4cccc(CO)c4)cc3)cc12] | 3-(6-chloro-5-(3′- (hydroxymethyl)-[1,1′- biphenyl]-4-yl)-1H-indazol-3- yl)propanoic acid |
| 62 | 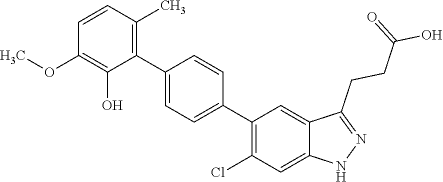 [SMILES: COc1ccc(C)c(-c2ccc(-c3cc4c(CCC(=O)O)n[nH]c4cc3Cl)cc2)c1O] | 3-(6-chloro-5-(2′-hydroxy-3′- methoxy-6′-methyl-[1,1′- biphenyl]-4-yl)-1H-indazol-3- yl)propanoic acid |
| 63 | 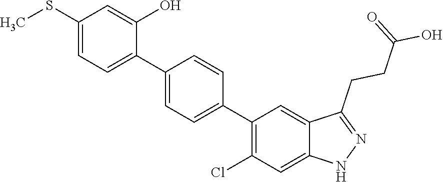 [SMILES: CSc1ccc(-c2ccc(-c3cc4c(CCC(=O)O)n[nH]c4cc3Cl)cc2)c(O)c1] | 3-(6-chloro-5-(2′-hydroxy-4′- (methylthio)-[1,1′-biphenyl]-4- yl)-1H-indazol-3-yl)propanoic acid |
| 64 | 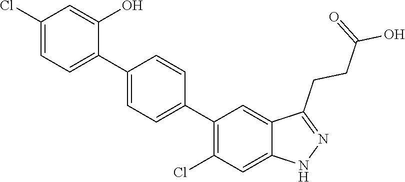 [SMILES: O=C(O)CCc1n[nH]c2cc(Cl)c(-c3ccc(-c4ccc(Cl)cc4O)cc3)cc12] | 3-(6-chloro-5-(4′-chloro-2′- hydroxy-[1,1′-biphenyl]-4-yl)- 1H-indazol-3-yl)propanoic acid |
| 65 | 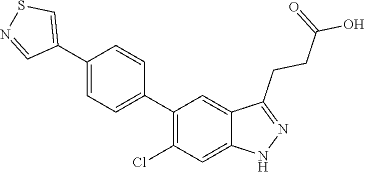 [SMILES: O=C(O)CCc1n[nH]c2cc(Cl)c(-c3ccc(-c4cnsc4)cc3)cc12] | 3-(6-chloro-5-(4-(isothiazol-4- yl)phenyl)-1H-indazol-3- yl)propanoic acid |
| 66 | 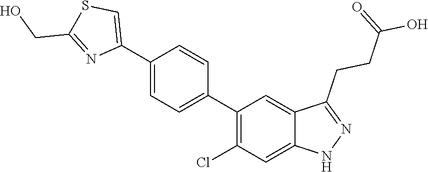 [SMILES: O=C(O)CCc1n[nH]c2cc(Cl)c(-c3ccc(-c4csc(CO)n4)cc3)cc12] | 3-(6-chloro-5-(4-(2- (hydroxymethyl)thiazol-4- yl)phenyl)-1H-indazol-3- yl)propanoic acid |
| 67 | 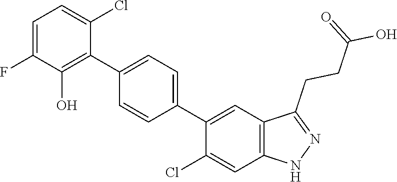 [SMILES: O=C(O)CCc1n[nH]c2cc(Cl)c(-c3ccc(-c4c(Cl)ccc(F)c4O)cc3)cc12] | 3-(6-chloro-5-(6′-chloro-3′- fluoro-2′-hydroxy-[1,1′- biphenyl]-4-yl)-1H-indazol-3- yl)propanoic acid |
| 68 | 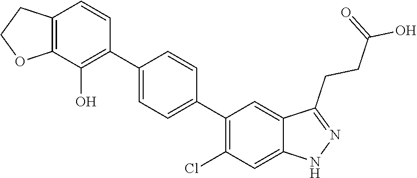 [SMILES: O=C(O)CCc1n[nH]c2cc(Cl)c(-c3ccc(-c4ccc5c(c4O)OCC5)cc3)cc12] | 3-(6-chloro-5-(4-(7-hydroxy- 2,3-dihydrobenzofuran-6- yl)phenyl)-1H-indazol-3- yl)propanoic acid |
| 69 | 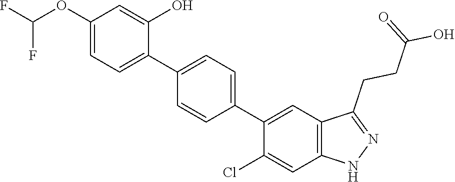 [SMILES: O=C(O)CCc1n[nH]c2cc(Cl)c(-c3ccc(-c4ccc(OC(F)F)cc4O)cc3)cc12] | 3-(6-chloro-5-(4′- (difluoromethoxy)-2′-hydroxy- [1,1′-biphenyl]-4-yl)-1H- indazol-3-yl)propanoic acid |
| 70 | 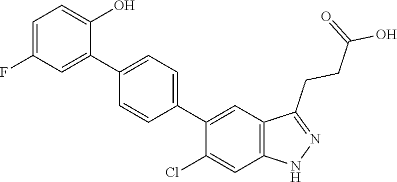 [SMILES: O=C(O)CCc1n[nH]c2cc(Cl)c(-c3ccc(-c4cc(F)ccc4O)cc3)cc12] | 3-(6-chloro-5-(5′-fluoro-2′- hydroxy-[1,1′-biphenyl]-4-yl)- 1H-indazol-3-yl)propanoic acid |
| 71 | 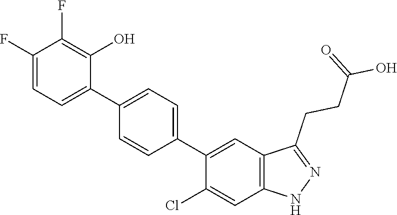 [SMILES: O=C(O)CCc1n[nH]c2cc(Cl)c(-c3ccc(-c4ccc(F)c(F)c4O)cc3)cc12] | 3-(6-chloro-5-(3′,4′-difluoro-2′- hydroxy-[1,1′-biphenyl]-4-yl)- 1H-indazol-3-yl)propanoic acid |
| 72 | 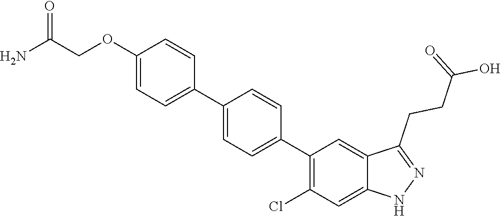 [SMILES: NC(=O)COc1ccc(-c2ccc(-c3cc4c(CCC(=O)O)n[nH]c4cc3Cl)cc2)cc1] | 3-(5-(4′-(2-amino-2- oxoethoxy)-[1,1′-biphenyl]-4- yl)-6-chloro-1H-indazol-3- yl)propanoic acid |
| 73 | 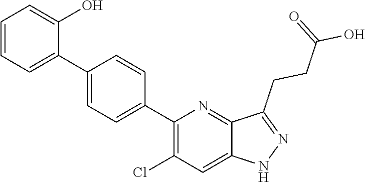 [SMILES: O=C(O)CCc1n[nH]c2cc(Cl)c(-c3ccc(-c4ccccc4O)cc3)nc12] | 3-(6-chloro-5-(2′-hydroxy-[1,1′- biphenyl]-4-yl)-1H- pyrazolo[4,3-b]pyridin-3- yl)propanoic acid |
| 74 | 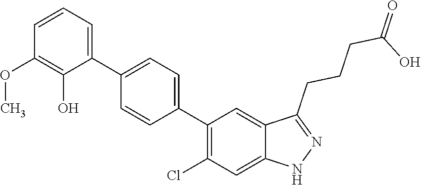 [SMILES: COc1cccc(-c2ccc(-c3cc4c(CCCC(=O)O)n[nH]c4cc3Cl)cc2)c1O] | 4-(6-chloro-5-(2′-hydroxy-3′- methoxy-[1,1′-biphenyl]-4-yl)- 1H-indazol-3-yl)butanoic acid |
| 75 | 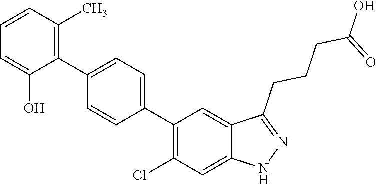 [SMILES: Cc1cccc(O)c1-c1ccc(-c2cc3c(CCCC(=O)O)n[nH]c3cc2Cl)cc1] | 4-(6-chloro-5-(2′-hydroxy-6′- methyl-[1,1′-biphenyl]-4-yl)-1H- indazol-3-yl)butanoic acid |
| 76 | 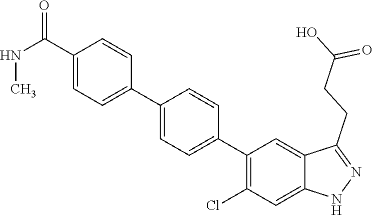 [SMILES: CNC(=O)c1ccc(-c2ccc(-c3cc4c(CCC(=O)O)n[nH]c4cc3Cl)cc2)cc1] | 3-(6-chloro-5-(4′- (methylcarbamoyl)-[1,1′- biphenyl]-4-yl)-1H-indazol-3- yl)propanoic acid |
| 77 | 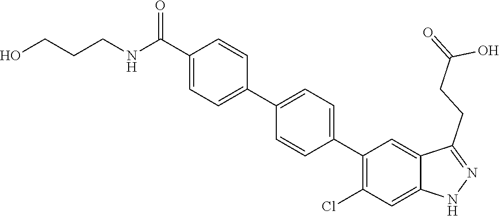 [SMILES: O=C(O)CCc1n[nH]c2cc(Cl)c(-c3ccc(-c4ccc(C(=O)NCCCO)cc4)cc3)cc12] | 3-(6-chloro-5-(4′-((3- hydroxypropyl)carbamoyl)- [1,1′-biphenyl]-4-yl)-1H- indazol-3-yl)propanoic acid |
| 78 | 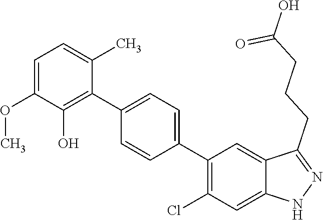 [SMILES: COc1ccc(C)c(-c2ccc(-c3cc4c(CCCC(=O)O)n[nH]c4cc3Cl)cc2)c1O] | 4-(6-chloro-5-(2′-hydroxy-3′- methoxy-6′-methyl-[1,1′- biphenyl]-4-yl)-1H-indazol-3- yl)butanoic acid |
| 79 | 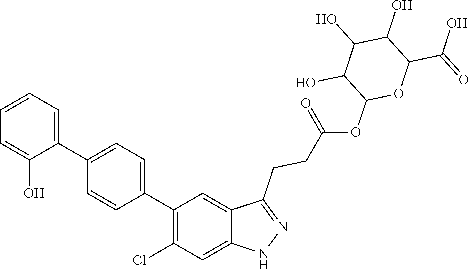 [SMILES: O=C(CCc1n[nH]c2cc(Cl)c(-c3ccc(-c4ccccc4O)cc3)cc12)OC1OC(C(=O)O)C(O)C(O)C1O] | 6-((3-(6-chloro-5-(2′-hydroxy- [1,1′-biphenyl]-4-yl)-1H- indazol-3-yl)propanoyl)oxy)- 3,4,5-trihydroxytetrahydro-2H- pyran-2-carboxylic acid |
| 80 | 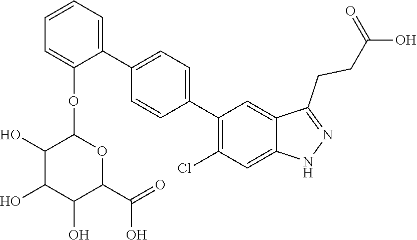 [SMILES: O=C(O)CCc1n[nH]c2cc(Cl)c(-c3ccc(-c4ccccc4OC4OC(C(=O)O)C(O)C(O)C4O)cc3)cc12] | 6-((4′-(3-(2-carboxyethyl)-6- chloro-1H-indazol-5-yl)-[1,1′- biphenyl]-2-yl)oxy)-3,4,5- trihydroxytetrahydro-2H-pyran- 2-carboxylic acid |

### Example 2: In Vitro Activity of AMPK-Activating Compounds

### Expression and Purification of AMPK111

A tricistronic AMPK expression construct was prepared, which included open reading frames encoding the full-length α1, β1 and δ1 subunits of human AMPK with a ribosome-binding site (RBS) ahead of each coding region. The construct was subcloned into pET-14b expression vector (Novagen, Madison, Wisconsin) using standard molecular biology techniques. AMPK tricistronic construct was transformed into E. coli BL21-CodonPlus™ (DE3)-RIPL strain (Stratagene), and transformants were selected on LB (Luria-Bertani) agar plates containing ampicillin (100 μg/mL). Ten liters of LB medium (MP Biomedical LB broth #11-3002-032) containing 100 μg/mL carbenicillin was inoculated with 100 mL E. coli shake flask culture (BL-21, pET-14b, AMPK 111) in a BF4 10 L working volume bioreactor (New Brunswick Scientific Co.) at 37° C., 600 rpm, 6 L/minute aeration. Optical density sample measurements were made on an UltroSpec 2000 spectrophotometer (Pharmacia Biotech) at 600 nm.

When the cell density reached ˜0.9OD, the temperature was reduced to 18° C. and the culture was induced at 18° C. with 0.1 mM Isopropylthiogalactoside (IPTG). The cell paste was collected at ˜18 hours post induction by refrigerated continuous flow centrifugation (Heraeus, rotor #8575) at 15,000 rpm at 4° C. The cell pellets were aliquoted into four portions, flash frozen in liquid nitrogen and were stored at −80° C. until purification. For purification, frozen cell paste was thawed and resuspended in 50 mL lysis buffer (50 mM Tris, pH 8.0, 150 mM NaCl, 10% glycerol, 2 mM Tris-2-carboxyethyl phosphine (TCEP), 20 mM imidazole and 0.001% Triton X-100). After sonication, insoluble material was removed by centrifugation at 15,000 rpm in a Sorvall® RC5 plus centrifuge for 30 min at 4° C. and the supernatant was loaded onto a 5 mL HisTrap™ HP column (GE Healthcare, Piscataway, NJ) and washed with five column volumes of lysis buffer. Bound proteins were eluted using an elution buffer containing 300 mM imidazole. Fractions containing AMPK subunits were pooled based on SDS-10% PAGE analysis and dialyzed overnight in dialysis buffer (50 mM Tris, pH 8.0, 150 mM NaCl, 10% glycerol, 2 mM TCEP, and 0.001% Triton X-100). The purified AMPK was phosphorylated on its activation loop Thr 172 by incubating 1.0 μM AMPK complex in the presence of 200 nM CaMKKB (calmodulin-dependent protein kinase B obtained from the University of Dundee) in phosphorylation buffer for 30 min at 30° C. The phosphorylated AMPK complex was re-purified on HisTrap™ HP column as before, dialyzed overnight in dialysis buffer. The phosphorylated AMPK complex was further purified by gel filtration chromatography with a Superdex 200 HiLoad 16/60 column (GE Healthcare) in SEC buffer (50 mM Tris, pH 8.0, 150 mM NaCl, 10% glycerol, 2 mM TCEP, and 0.001% Triton X-100). The final samples were stored at −20° C. with 25% glycerol.

### Expression and Purification of AMPK221

A tricistronic AMPK expression construct was designed that included open reading frames encoding the full-length α2, β2 and δ1 subunits of human AMPK with a ribosome-binding site (RBS) ahead of each coding region. The construct was subcloned into pET-14b expression vector (Novagen, Madison, Wisconsin) using standard molecular biology techniques. AMPK tricistronic construct was transformed into E. coli BL21-CodonPlus™ (DE3)-RIPL strain (Stratagene) and transformants were selected on LB (Luria-Bertani) agar plates containing ampicillin (100 μg/mL). Ten liters of LB medium (MP Biomedical LB broth #11-3002-032) containing 100 μg/mL carbenicillin was inoculated with 100 mL E. coli shake flask culture (BL-21, pET-14b, AMPK 221) in a BF4 10 L working volume bioreactor (New Brunswick Scientific Co.) at 37° C., 600 rpm, 6 L/minute aeration. Optical density sample measurements were made on an UltroSpec 2000 spectrophotometer (Pharmacia Biotech) at 600 nm.

When the cell density reached ˜0.9 OD, the temperature was reduced to 18° C. and the culture was induced at 18° C. with 0.1 mM Isopropylthiogalactoside (IPTG). The cell paste was collected at ˜18 hours post induction by refrigerated continuous flow centrifugation (Heraeus, rotor #8575) at 15,000 rpm at 4° C. The cell pellets were aliquoted into four portions, flash frozen in liquid nitrogen and were stored at −80° C. until purification. For purification, frozen cell paste was thawed and resuspended in 50 mL lysis buffer (50 mM Tris, pH 8.0, 150 mM NaCl, 10% glycerol, 2 mM Tris-2-carboxyethyl phosphine (TCEP), 20 mM imidazole and 0.001% Triton X-100). After sonication, insoluble material was removed by centrifugation at 15,000 rpm in a Sorvall® RC5 plus centrifuge for 30 min at 4° C. and the supernatant was loaded onto a 5 mL HisTrap™ HP column (GE Healthcare, Piscataway, NJ) and washed with five column volumes of lysis buffer. Bound proteins were eluted using an elution buffer containing 300 mM imidazole. Fractions containing AMPK subunits were pooled based on SDS-10% PAGE analysis and dialyzed overnight in dialysis buffer (50 mM Tris, pH 8.0, 150 mM NaCl, 10% glycerol, 2 mM TCEP, and 0.001% Triton X-100). The purified AMPK was phosphorylated on its activation loop Thr 172 by incubating 1.0 μM AMPK complex in the presence of 200 nM CaMKKB (calmodulin-dependent protein kinase B) obtained from the University of Dundee) in phosphorylation buffer for 30 min at 30° C. The phosphorylated AMPK complex was re-purified on HisTrap™ HP column as before, dialyzed overnight in dialysis buffer. The phosphorylated AMPK complex was further purified by gel filtration chromatography with a Superdex 200 HiLoad 16/60 column (GE Healthcare) in SEC buffer (50 mM Tris, pH 8.0, 150 mM NaCl, 10% glycerol, 2 mM TCEP, and 0.001% Triton X-100). The final samples were stored at −20° C. with 25% glycerol.

### Expression and Purification of PP2A

The coding sequence for human recombinant Protein Phosphatase 2A catalytic subunit (PPP2CA; 308 aa, β67775, AA2-309) with an N-terminal 2×FLAG tag and a TEV protease site was synthesized and subcloned into the pFastBac Dual expression vector (Thermo Fisher, 10712024) under the PoIH promoter and the Protein Phosphatase 2A regulatory subunit (PPP2R1A; 508 aa, β30153, AA2-589) with an N-terminal His tag. A TEV protease site was synthesized and subcloned into the same vector under the β10 promoter. The vector was used to make baculovirus using the Bac-to-Bac System (Thermo Fisher) which was subsequently used to express protein in Sf9 cells (Expression Systems, 94-001 F). The Sf9 cells were cultured in ESF 921 medium (Expression Systems, 96-001-01) at a 3 L volume in a sterile 5 L Thomson Optimum Growth Flask with a vent cap (Thomson Instrument Company, 931116) at 27° C. while shaking at 115 rpm with a 2-inch shaking diameter. P0 virus was used at 10 mls/L to infect cells at a density ˜2.5×106 vc/ml at >95% viability. The harvest time (71 hours post infection) was indicated by percent cell viability (˜80%) and increased cell diameter (>3 micron). The cell paste was collected by centrifugation in a Thermo Sorvall RC 3BP+ Centrifuge at 5000×g and frozen at −80° C.

For purification, the 3 L of culture worth of cell paste was resuspended in 175 mL Lysis/wash buffer (50 mM Tris pH 8.0, 300 mM NaCl, 10% glycerol, 1 mM TCEP). Cells were lysed by micro fluidization at 15,000 PSI for 3 passes and clarified by centrifugation 30,000×g. 5 mL of FLAG resin was equilibrated with lysis buffer. Equilibrated FLAG resin was added to the supernatant and allowed to batch bind 6 hours. The protein was then washed with 20 volumes of lysis buffer and eluted off of the resin with 7 mL of elution buffer (50 mM Tris pH 8.0, 300 mM NaCl, 10% glycerol, 0.25 mg/ml FLAG peptide. Eluted fractions were analyzed by SDS-PAGE, Mass Spec, and PP2a activity. Total protein concentration was determined by Superdex 200 16-60, protein determined to be 0.149 mg/mL.

### Biochemical Profiling of AMPK Activators by AMPK111

The biochemical EC₅₀ (half-maximal concentration required for full activation) of compounds for the activation of AMPK was evaluated by HTRF assay using LANCE Ultra ULight-Acetyl-CoA Carboxylase (SAMS) peptide (commercially available, Perkin Elmer catalog TRF0133-M). 5 μL of 0.3 nM phosphorylated AMPK 111 (isolation detailed above) diluted in assay buffer (50 mM HEPES, 1 mM EGTA, 10 mM MgCl2, 0.25 mM DTT, 0.01% Tween-20, 0.01% BSA (pH 7.5), was added to white 384 well plates (Corning catalog number 3824) containing 0.075 μL of test compound, (solubilized and serially diluted in DMSO, in a 11 point, %-log dilution series, tested in duplicate).

Plates were spun at 1000 RPM for 10 seconds. Following a fifteen-minute room temperature incubation, 5 μL of 30 nM protein phosphatase PP2A (isolation detailed above) diluted in the assay buffer was added to the plate, to dephosphorylate pThr172 of AMPK. Plates were spun at 1000 RPM for 10 seconds. After incubation for 120 minutes, 5 μL of substrate mixture containing 60 nM okadaic acid (Tocris catalog number 1136), 150 nM SAMS peptide (Perkin Elmer catalog TRF0133-M), and 60 μM ATP (Teknova catalog number A1204) diluted in assay buffer, was added to the plate. Plates were spun at 1000 RPM for 10 seconds. The reaction was terminated after 60 minutes incubation at room temperature, by the addition of 5 μL of stop and detection cocktail, which consisted of 1× Perkin Elmer Lance buffer (Perkin Elmer catalog number CR97-100), 40 mM EDTA (Thermo Fisher catalog number BP2482-100), and 2 nM Eu-anti-Acetyl CoA Carboxylase [pSer70] antibody (Perkin Elmer, TRF0208-M). Plates were spun at 1000 RPM for 10 seconds. Plates were incubated for 1 hour, then read on an Envision reader with settings for TR-FRET Ratio=10,000× (fluorescence intensity 665 nM/fluorescence intensity 615 nM). EC₅₀ values were determined from this data using a 4-parameter fit algorithm and are presented in Table 2.

### Biochemical Profiling of AMPK Activators by AMPK221

The biochemical EC₅₀ (half-maximal concentration required for full activation) of compounds for the activation of AMPK was evaluated by HTRF assay using LANCE Ultra ULight-Acetyl-CoA Carboxylase (SAMS) peptide (commercially available, Perkin Elmer catalog TRF0133-M). 5 μL of 0.3 nM phosphorylated AMPK 221(isolation detailed above) diluted in assay buffer (50 mM HEPES, 1 mM EGTA, 10 mM MgCl2, 0.25 mM DTT, 0.01% Tween-20, 0.01% BSA (pH 7.5) was added to white 384 well plates (Corning catalog number 3824) containing 0.075 μL of test compound (solubilized and serially diluted in DMSO, in a 11 point, %-log series and tested in duplicate).

Plates were spun at 1000 RPM for 10 seconds. Following a fifteen-minute room temperature incubation, 5 μL of 15 nM protein phosphatase PP2A (isolation detailed above) diluted in the assay buffer was added to the plate to dephosphorylate pThr172 of AMPK. Plates were spun at 1000 RPM for 10 seconds. After incubation for 120 minutes, 5 μL of substrate mixture containing 30 nM okadaic acid (Tocris catalog number 1136), 150 nM SAMS peptide (Perkin Elmer catalog TRF0133-M) and 240 μM ATP (Teknova catalog number A1204), diluted in assay buffer, was added to the plate. Plates were spun at 1000 RPM for 10 seconds. The reaction was terminated after 60 minutes incubation at room temperature by the addition of 5 μL of stop and detection cocktail, which consisted of 1× Perkin Elmer Lance buffer (Perkin Elmer catalog number CR97-100), 40 mM EDTA (Thermo Fisher catalog number BP2482-100), and 2 nM Eu-anti-Acetyl CoA Carboxylase [pSer70] antibody (Perkin Elmer, TRF0208-M). Plates were spun at 1000 RPM, 10 seconds. Plates were incubated for 1 hour, then read on an Envision reader with settings for TR-FRET Ratio=10,000× (fluorescence intensity 665 nM/fluorescence intensity 615 nM). EC₅₀ values were determined from this data using a 4-parameter fit algorithm and are presented in Table 2.

TABLE 2
Biochemical profiling of AMPK activators
by AMPK 111 and AMPK221

| | |
|---|---|
| AMPK 111 EC₅₀ | AMPK 221 |

| Example | nM | eMax (%) | nM | eMax (%) |
|---|---|---|---|---|
| 1 | 47 | 75 | 88 | 57 |
| 2 | 29 | 80 | 34 | 66 |
| 3 | 189 | 83 | 528 | 61 |
| 4 | 62 | 74 | 665 | 34 |
| 5 | 89 | 59 | 37 | 62 |
| 6 | 6 | 64 | 4 | 53 |
| 7 | 90 | 71 | 359 | 57 |
| 8 | 26 | 75 | 38 | 56 |
| 9 | 11 | 90 | 41 | 68 |
| 10 | 29 | 76 | 48 | 55 |
| 11 | 53 | 62 | 60 | 44 |
| 12 | 16 | 72 | 62 | 56 |
| 13 | 21 | 86 | 69 | 67 |
| 14 | 45 | 74 | 72 | 65 |
| 15 | 35 | 75 | 76 | 52 |
| 16 | 37 | 86 | 81 | 64 |
| 17 | 34 | 80 | 83 | 49 |
| 18 | 12 | 84 | 96 | 64 |
| 19 | 54 | 79 | 144 | 34 |
| 20 | 16 | 82 | 97 | 61 |
| 21 | 151 | 65 | 97 | 56 |
| 22 | 111 | 74 | 100 | 59 |
| 23 | 2 | 71 | 5 | 56 |
| 24 | 19 | 86 | 106 | 48 |
| 25 | 144 | 71 | 107 | 61 |
| 26 | 107 | 77 | 114 | 66 |
| 27 | 7 | 87 | 125 | 62 |
| 28 | 277 | 73 | 136 | 63 |
| 29 | 46 | 72 | 162 | 63 |
| 30 | 36 | 80 | 173 | 65 |
| 31 | 57 | 84 | 190 | 61 |
| 32 | 136 | 71 | 198 | 41 |
| 33 | 301 | 57 | 196 | 57 |
| 34 | 228 | 68 | 197 | 50 |
| 35 | 137 | 67 | 198 | 47 |
| 36 | 45 | 68 | 200 | 49 |
| 37 | 464 | 70 | 205 | 52 |
| 38 | 95 | 67 | 209 | 64 |
| 39 | 109 | 69 | 218 | 59 |
| 40 | 92 | 85 | 306 | 68 |
| 41 | 70 | 66 | 235 | 65 |
| 42 | 291 | 68 | 244 | 64 |
| 43 | 51 | 80 | 248 | 62 |
| 44 | 85 | 79 | 204 | 52 |
| 45 | 123 | 82 | 263 | 66 |
| 46 | 20 | 82 | 267 | 59 |
| 47 | 113 | 83 | 282 | 59 |
| 48 | 365 | 43 | 367 | 9 |
| 49 | 96 | 74 | 325 | 61 |
| 50 | 153 | 76 | 337 | 55 |
| 51 | 312 | 71 | 355 | 22 |
| 52 | 6 | 85 | 19 | 65 |
| 53 | 218 | 69 | 644 | 55 |
| 54 | 217 | 61 | 1006 | 64 |
| 55 | 208 | 45 | 389 | 38 |
| 56 | 363 | 48 | 477 | 18 |
| 57 | 103 | 75 | 426 | 63 |
| 58 | 215 | 76 | 573 | 57 |
| 59 | 609 | 30 | 546 | 13 |
| 60 | 241 | 53 | 532 | 21 |
| 61 | 692 | 55 | 645 | 34 |
| 62 | 25 | 72 | 14 | 57 |
| 63 | 281 | 56 | 565 | 64 |
| 64 | 203 | 76 | 617 | 48 |
| 65 | 263 | 79 | 795 | 58 |
| 66 | 365 | 74 | 1087 | 63 |
| 67 | 69 | 85 | 953 | 63 |
| 68 | 29 | 71 | 19 | 69 |
| 69 | 480 | 73 | 1151 | 58 |
| 70 | 285 | 74 | 1086 | 49 |
| 71 | 279 | 73 | 994 | 60 |
| 72 | 99 | 103 | 1831 | 114 |
| 73 | 26 | 86 | 2115 | 30 |
| 74 | 92 | 80 | 66 | 69 |
| 75 | 74 | 75 | 229 | 63 |
| 76 | 6 | 67 | 13 | 53 |
| 77 | 70 | 66 | 82 | 58 |
| 78 | 93 | 78 | 87 | 66 |
| 79 | 39 | 67 | 29 | 27 |
| 80 | >21100 | | >21100 | |

### Example 3: Method of Preparing Extended-Release Formulation; Melt-Spray-Congeal Process

Compound 1 is a compound of Formula (I), a pharmaceutically acceptable salt, a tautomer, or a pharmaceutically acceptable salt of the tautomer thereof.

Approximately 50% of the matrix excipient was charged to an appropriately sized bin, then blended for at least 1 minute at 12 RPM in a turbula blender. Compound 1 was added to the bin. The release modifier was then added to the bin. The remaining matrix excipient was then added to the bin. The resulting mixture was blended for 5 minutes at 12 RPM. The resulting blend was then processed by a Comil at 1000 RPM using a 156R screen. The milled blend was subjected to additional blending for at least 15 minutes at 12 RPM.

The milled blend was then transferred to a gravimetric powder feeder. The powder feeder delivered the blend to a 18 mm twin-screw extruder at a rate of 4.0±0.5 kg/hr. The twin screw extruder, operating with screw speed 180±50 RPM, conveyed the blend while heating from 30±15° C. (powder entry zone) to 80±5° C. (extrudate exit). As the blend was conveyed and heated, the matrix component melted, and Compound 1 was suspended in the melt. The resulting melt suspension was conveyed from the twin screw extruder to a spinning disk. The spinning disk atomized the melt suspension to generate spherical particles that rapidly congealed in the collection bag that was maintained at ambient temperatures. The disk temperature range and target for stearyl alcohol matrices was 80±5° C. The disk temperature range and target for polyglyceryl-4-stearate and polyglyceryl-6-palmate matrices was 90±5° C. The disk speed range studied was 2,500 to 4,000 RPM. The selected disk speed was 3,250±250 RPM.

The congealed microspheres were then collected and passed through a high throughout screener. The screen size for removing oversized particles was 425 microns, and the screener frequency was 70 hz. The screened microspheres were then annealed on trays at 40±2° C. and 75±5% relative humidity. The microspheres were annealed for about 3 days. Table 3 shows formulations prepared using the described melt-spray-congeal method.

TABLE 3
Formulations prepared using a melt-spray-congeal process

| Component | Formulation 1 | Formulation 2 | Formulation 3 |
|---|---|---|---|
| Stearyl Alcohol | 68 | 76 | 72 |
| Compound 1 | 20 | 20 | 20 |
| Crospovidone XL-10 | 12 | 4 | 8 |
| Total | 100 | 100 | 100 |

### Example 4: Method of Making Coated Microspheres (Microcapsules)

A coating suspension was prepared by first weighing out the Eudragit L30D-55 and Eudragit FS30D in an appropriately sized container. The PlasACRYL T20 was then weighed out into a separate container. While mixing, the PlasACRYL T20 was slowly added to the Eudragit mixture. Purified water was weighed out into a separate container. While mixing, the purified water was slowly added to the Eudragit and PlasACRYL mixture. The final coating suspension was mixed for a minimum of 30 minutes prior to coating the microsphere particles. The coating solution was passed through a 180 (150-200) micron screen immediately prior to coating. The suspension was stirred continuously after preparation and throughout the duration of the coating run. Table 4 shows the coater parameters used to prepare the coated microspheres. Table 5 shows the processing conditions used to prepare the coated microspheres.

TABLE 4
Coater parameters

| Component/Parameter | Setting | |
|---|---|---|
| Instrument | Glatt GPCG2 | |
| Product Bowl Diameter | 4″ | |
| Retention Screen Size | 75 | micron |
| Air Distribution Plate | B-type | |
| Column Gap | 15-25 | mm |
| Product Filter | T165P (20 um) | |
| Nozzle Bore | 0.8 | mm |

| | |
|---|---|
| Spray Tubing Size | L/S 16 |

TABLE 5
Processing conditions

| Process Parameter | Setting | |
|---|---|---|
| Spray Rate Target | 8.0 | g/min |
| Product Temperature Target | 27° | C. |
| Inlet Air Temperature | 45° | C. |
| Inlet Air Flow | 75 | m³/hr |
| Atomization Air Pressure | 2.5 | bar |

The batch quantity of microsphere cores were charged to the product chamber. The chamber was preheated under fluidization until the pre-heat conditions were achieved. The suspension pump was started at the target spray rate. The inlet air temperature was increased to the coating target. Routine samples were taken to assess attrition and/or agglomeration. A sieve was used to monitor process agglomeration as needed. The inlet air temperature was adjusted throughout the run to maintain the target product temperature. The process airflow was adjusted as needed to maintain proper fluidization of the microspheres as the coating level increased. Upon coating completion, the coated microspheres were transferred from the product chamber to a collection container.

### Example 5: Characterization of Extended-Release Microsphere Particles

Determining particle sizes of extended-release microspheres by laser diffraction; Microsphere particle size was determined by laser diffraction on a Sympatec Helos laser diffraction instrument equipped with Vibri vibrating feeder unit (Sympatec GmbH). Samples were dispersed at a pressure of 1.0 bar, and the measurement was performed while optical concentration was above 1%. Lenses were selected based on the approximate size range of particles observed in optical images, which was typically the R5 with measuring range of 4.5-875 microns. Particle size was calculated by Fraunhofer theory in WINDOX 5.9 or PAQXOS 5.0.1 software.

In vitro dissolution using USP II apparatus: In vitro dissolution data was obtained in 0.05 M sodium phosphate buffer, pH 6.8, fasted simulated state intestinal fluid (FaSSIF), fed simulated state intestinal fluid, fasted simulated state colonic fluid (FaSSCoF), and fasted simulated state gastric fluid (FaSSGF). The in vitro dissolution of the prepared formulations was performed using a USP II dissolution apparatus (paddles) and commercially available dissolution media. The stir rate and temperature were fixed at 50 RPM and 37° C., respectively. Absorbance measurements were taken by fiber optic probes placed within each vessel. Dissolution data was processed using Pion AuPRO™ or Distek Opt-Diss software.

Assessment of solid crystalline form by PXRD: Powder X-ray diffraction (PXRD) for Compound 1 and microsphere formulations was performed on a Bruker AXS D8 Endeavor diffractometer with Cu radiation source and PSD-Lynx Eye detector. The X-ray tube voltage and amperage were set to 40 kV and 40 mA, respectively. Samples were placed in low background silicon sample wafers, and data were collected at the Cu wavelength (CuKα̅=1.5418λ) in the Theta-Theta goniometer from 3.0 to 40.0 degrees 2-Theta, with a step size of 0.016° and step time of 0.4 seconds. Data collection was performed with Bruker DIFFRAC Plus software, and analysis was performed in Bruker DIFFRAC.EVA software.

### Example 6: Impact of Formulation and Process Variables on Microsphere Particle Size and In Vitro Dissolution Release Profile for Uncoated Microspheres

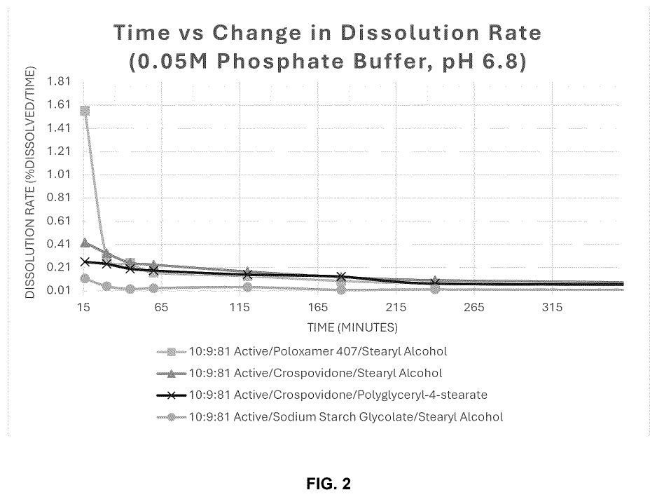

Effect of microsphere matrix components: The effect of microsphere matrix components was studied using formulations comprising stearyl alcohol or polyglyceryl-4-stearate. FIG. 2 illustrates differences in the time vs change in dissolution rate of a formulation comprising: 1) 10:9:81 Compound 1/poloxamer 407/stearyl alcohol; 2) 10:9:81 Compound 1/crospovidone/stearyl alcohol; 3) 10:9:81 Compound 1/crospovidone/polyglyceryl-4-stearate; and 4) 10:9:81 Compound 1/sodium starch glycolate/stearyl alcohol. FIG. 2 shows that the stearyl alcohol formulation containing comparable quantities of crospovidone and Compound 1 exhibited faster dissolution rates than the equivalent polyglyceryl-4-stearate containing formulation, which was preferable for the current formulation.

Effect of microsphere release modifier: Use of Crospovidone as a release modifier resulted in more consistent release rates than Poloxamer 407. The rapid initial release rate mediated by poloxamer 407 resulted in dissolution of >20% Compound 1 in less than 15 minutes. In contrast, use of sodium starch glycolate resulted in a negligible release rate, such that <30% of Compound 1 was dissolved after 30 hours. FIG. 2 illustrates differences in the time vs change in dissolution rate of a formulation comprising: 1) 10:9:81 Compound 1/poloxamer 407/stearyl alcohol; 2) 10:9:81 Compound 1/crospovidone/stearyl alcohol; 3) 10:9:81 Compound 1/crospovidone/polyglyceryl-4-stearate; and 4) 10:9:81 Compound 1/sodium starch glycolate/stearyl alcohol. Of the release modifiers studied, crospovidone demonstrated the ability to consistently modulate release at a desired in vitro release rate. Use of Poloxamer 407 resulted in an undesirably rapid initial in vitro release rate, and use of Sodium Starch Glycolate resulted in an undesirably slow release rate.

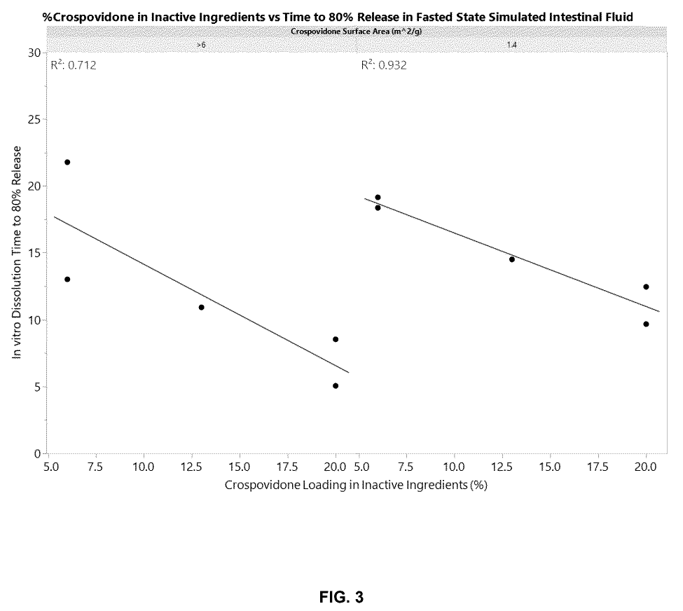

Effect of crospovidone grade on in vitro dissolution performance: Formulations containing crospovidone with a surface area of 1.4 m²/g demonstrated a clear relationship between Crospovidone content (% w/w) in inactive ingredients and in vitro dissolution Time to 80% Release in Fasted State Simulated Intestinal Fluid (FaSSIF). A similar relationship was observed for formulations containing crospovidone with a surface area of >6 m²/g, but the correlation coefficient for the relationship was reduced, suggesting that such formulations produce less predictable results in terms of in vitro dissolution performance. FIG. 3 shows the crospovidone content (% w/w) in inactive ingredients vs time to 80% release in fasted state simulated intestinal fluid for a formulation comprising crospovidone with a surface area of 1.4 m²/g or >6 m²/g. Of the two crospovidone samples tested in more than one formulation, crospovidone with a surface area of 1.4 m²/g demonstrated a clear and predictable relationship between crospovidone content (% w/w) in inactive ingredients and in vitro dissolution Time to 80% Release in Fasted State Simulated Intestinal Fluid (FaSSIF).

Effect of crospovidone loading on in vitro dissolution performance: The greater the crospovidone content (% w/w) in the formulation, the faster the formulation achieved 80% dissolved in vitro. The F-test P value suggested the effect of formulation crospovidone content (% w/w) on in vitro dissolution time to 80% release was significant. FIG. 3 shows crospovidone (% w/w) in inactive ingredients vs time to 80% release in fasted state simulated intestinal fluid for a formulation comprising crospovidone with a surface area of 1.4 m²/g. The fraction of formulation inactive ingredients comprised of crospovidone significantly impacted the in vitro release rate of the dosage form.

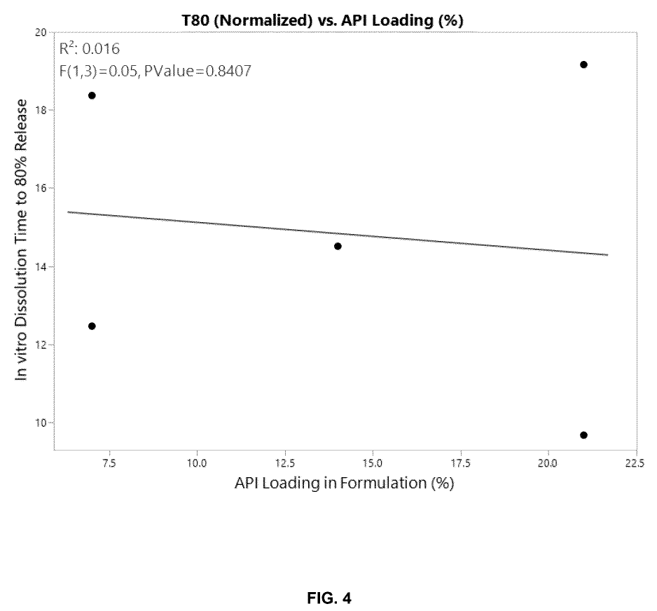

Effect of Compound 1 loading on in vitro dissolution performance: The poor fit of the linear trend line drawn for Compound 1 (% w/w) vs in vitro dissolution time to 80% release suggests the amount of Compound 1 in the formulation (between 7-21% w/w) did not significantly impact in vitro dissolution time to 80% release. This was further supported by the F-test P value. FIG. 4 shows Compound 1 loading (% w/w) vs in vitro dissolution time to 80% release. Changing the formulation API content (7-21%) had little or no effect on dosage form in vitro release rate.

### Example 7: Coated Microsphere Formulations

Impact of methacrylic acid coatings on in vitro dissolution: A 25:75 L30D55:FS30D:L30D55 coating, when applied to achieve 35% coat weight, limited release of the Compound 1 to less than approximately 10% over two hours in Fed Simulated State Intestinal Fluid (FeSSIF) at pH 4.5 and Fasted Simulated State Intestinal Fluid (FaSSIF) at pH 6.5. The 100% FS30D coating, when applied to achieve 35% coat weight, limited release of the Compound 1 to less than approximately 5% over two hours in FeSSIF, pH 4.5 and FaSSIF, pH 6.5. Over the same time frame, the uncoated formulation released approximately 50% of Compound 1 in FeSSIF, pH 4.5 and approximately 35% in FaSSIF, pH 6.5.

Microspheres with 35% coat weight (both 100% FS30D and 25:75 L30-D55:FS30D) exhibited reduced in vitro release at two hours relative to uncoated microspheres when tested in FeSSIF media at pH 4.5 (simulated fed state) and FaSSIF (simulated fasted state) media at pH 6.5. Microspheres with 35% coat weight (both 100% FS30D and 25:75 L30-D55:FS30D) exhibited prolonged release profiles relative to uncoated microspheres in FaSSIF (simulated fasted state) media at pH 6.8. Microspheres with 35% coat weight (both 100% FS30D and 25:75 L30-D55:FS30D) exhibit release profiles that are consistent with uncoated microspheres in Fasted Simulated State Colonic Fluid (FaSSCoF) at pH 7.8.

### Example 8: Uncoated Microsphere Formulations

Stearyl alcohol uncoated microspheres: Uncoated stearyl alcohol microsphere formulations are prepared with varying amounts of a compound of the disclosure, a pharmaceutically acceptable salt, a tautomer, or a pharmaceutically acceptable salt of the tautomer thereof; stearyl alcohol; and a disintegrant or pore-forming agent. The disintegrant or pore-forming agent is selected from povidone, methylcellulose, or hydroxypropylmethylcellulose (Hypromellose or HPMC). Compound 1 had a target particle sizes of D50 of from about 1 μm to about 50 μm and D90 of from about 10 μm to about 125 μm. Table 6 summarizes the component amounts of stearyl alcohol containing uncoated microspheres.

TABLE 6
Formulations of uncoated stearyl alcohol microspheres

| | | | Disintegrant | |
|---|---|---|---|---|
| | | | or pore- | |
| Compound 1 | | Stearyl | forming | |
| Particle Size | Compound 1 | Alcohol | agent | Intended |
| Range | Loading | Loading | loading | Character- |
| (D50 \| D90) | Range | Range | Range | ization |
| 1 μm \| 10 μm | 5-30% | 55-90% | 5-15% | Particle size, |
| to | | | | PXRD, Melt |
| 50 μm \| 125 μm | | | | viscosity, Melt |
| | | | | solubility, |
| | | | | biorelevant |
| | | | | in vitro |
| | | | | dissolution |

Polyglyceryl-4-stearate uncoated microspheres: Uncoated polyglyceryl-4-stearate microsphere formulations are prepared with varying amounts of a compound of the disclosure, a pharmaceutically acceptable salt, a tautomer, or a pharmaceutically acceptable salt of the tautomer thereof; stearyl alcohol; and a disintegrant or pore-forming agent. The disintegrant or pore-forming agent is selected from crospovidone, povidone, methylcellulose, or hydroxypropylmethylcellulose (Hypromellose or HPMC). Compound 1 had a target particle sizes of D50 of from about 1 μm to about 50 μm and D90 of from about 10 μm to about 125 μm. Table 7 summarizes the component amounts of polyglyceryl-4-stearate containing uncoated microspheres.

TABLE 7
Formulations of uncoated polyglyceryl-4-stearate microspheres

| | | | Disintegrant | |
|---|---|---|---|---|
| | | Poly- | or pore- | |
| Compound 1 | | glyceryl- | forming | |
| Particle Size | Compound 1 | 4-stearate | agent | Intended |
| Range | Loading | Loading | loading | Character- |
| (D50 \| D90) | Range | Range | Range | ization |
| 1 μm \| 10 μm | 5-30% | 55-90% | 5-15% | Particle size, |
| to | | | | PXRD, Melt |
| 50 μm \| 125 μm | | | | viscosity, |
| | | | | Melt |
| | | | | solubility, |
| | | | | in vitro |
| | | | | dissolution |
| | | | | in FaSSIF |

### Example 9: Coated Microsphere (Microcapsule) Formulations

An expanded pH-coating screening is performed for formulations comprising a compound of the disclosure, a pharmaceutically acceptable salt, a tautomer, or a pharmaceutically acceptable salt of the tautomer thereof; stearyl alcohol or polyglyceryl-4-stearate; and a release modifier. The expected coat weight gain is from about 10% to about 30%. The ration of coating components (L30D55:FS30D) is from about 50:50 to about 10:90. The microcapsules are characterized by particle size, PXRD, and biorelevant in vitro dissolution.

A screening for hot-melt coatings is performed for formulations comprising a compound of the disclosure, a pharmaceutically acceptable salt, a tautomer, or a pharmaceutically acceptable salt of the tautomer thereof; stearyl alcohol or polyglyceryl-4-stearate; and a release modifier. Application of a hydrophobic lipid coating impedes water permeation into the microsphere cores. The coat weight is from about 10% to about 30%. The coating comprises stearyl alcohol, polyglyceryl-4-stearate, polyglycerol-3-behenate, polyglyceryl-6-palmate, or a combination thereof. The microcapsules are characterized by particle size, PXRD, and biorelevant in vitro dissolution.

A screening for a rupturable coating is performed for formulations comprising a compound of the disclosure, a pharmaceutically acceptable salt, a tautomer, or a pharmaceutically acceptable salt of the tautomer thereof; stearyl alcohol or polyglyceryl-4-stearate; and a release modifier. The rupturable coating ruptures upon shape reformation or swelling of excipients present in the microsphere core or a coating layer applied to the core prior to application of the rupturable coating. The coat weight gain is from about 10% to about 30%. The primary coating component is ethyl cellulose, cellulose acetate, or a combination thereof. The microcapsules are characterized by particle size, PXRD, and biorelevant in vitro dissolution.

### Example 10: First in Human (FIH) Study Formulation

Uncoated microsphere formulations were prepared for first in human studies. The formulations comprised a compound of the disclosure, a pharmaceutically acceptable salt, a tautomer, or a pharmaceutically acceptable salt of the tautomer thereof; stearyl alcohol; and crospovidone. The microsphere formulations were manually packaged into capsules for oral administration. Table 8 summarizes two microsphere formulations prepared for FIH studies.

TABLE 8
FIH Study formulations

| | | | | | | | Time to |
|---|---|---|---|---|---|---|---|
| | | | | | | | 80% |
| | | | Stearyl | | | | release in |
| Formulation | Compound | Crospovidone | Alcohol | D50 | D4, 3 | D3, 2 | FaSSIF |
| F1 | 20% | 12% | 68% | 195.46 μm | 175-225 μm | 150-200 μm | Approx |
| | | | | | | | 10 hours |
| F2 | 20% | 7.2% | 72.8% | TBD | TBD | TBD | Approx |
| | | | | | | | 18 hours |

### Example 11: Amorphous form of 3-(6-Chloro-5-(2′-hydroxy-[1,1′-biphenyl]-4-yl)-1H-indazol-3-yl)propanoic acid

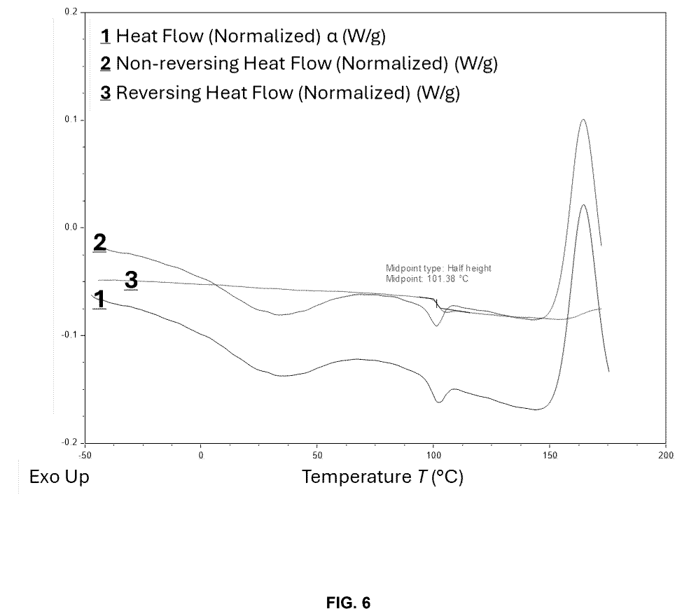

Preparation of amorphous Form 3: Approximately 150 mg of 3-(6-Chloro-5-(2′-hydroxy-[1,1′-biphenyl]-4-yl)-1H-indazol-3-yl)propanoic acid was added to a 100 mL round bottom flask. 30 mL of acetonitrile was added to the flask, and it was sonicated for 5 minutes. The flask was placed in a 40° C. water bath for 10 minutes until all the material was dissolved. 10 mL of water was added, and the material was again sonicated for 5 minutes. The sample was frozen in a bath of dry ice and acetone and freeze dried. Material was isolated after the removal of all ice. The PXRD diffractogram is shown in FIG. 6. The diffractogram was consistent with that of an amorphous material. A modulated DSC was performed to determine the glass transition temperature of the amorphous material at 0% RH. The glass transition of this material was 101° C.

Powder X-ray diffraction (PXRD): Powder X-ray diffraction analysis was conducted using a Bruker AXS D8 Endeavor diffractometer equipped with a Cu radiation source. The divergence slit was set at 10 mm continuous illumination. Diffracted radiation was detected by a PSD-Lynx Eye detector, with the detector PSD opening set at 4.1 degrees. The X-ray tube voltage and amperage were set to 40 kV and 40 mA respectively. In addition, the energy dispersive detector, a nickel filter was used to screen out unwanted wavelengths. Data was collected in the Theta-Theta goniometer at the Cu wavelength from 3.0 to 40.0 degrees 2-Theta using a step size of 0.02 degrees and a step time of 0.15 second. The antiscatter screen was set to a fixed distance of 1.5 mm. Samples were prepared by placing them in a silicon low background sample holder and rotated at 15/min during collection. Data were collected using Bruker DIFFRAC Plus software and analysis was performed by EVA diffract plus software. The sample holder used in a particular experiment is given by a codename within the filename: SD=small divot holder.

Modulated differential scanning calorimetry (mDSC): Modulated differential scanning calorimetry was conducted with a DSC measurements were performed with DSC 2500 (TA instruments) equipped with a refrigerated cooling accessory. The cell constant was determined using indium and temperature calibration was performed using indium and tin as standards. All the measurements were done under continuous dry nitrogen purge (50 mL/min). Approximately 1-5 mg of solid sample was weighed into a Tzero aluminum pan, sealed non-hermetically and heated from −50° C. to 250° C. at 2° C./min heating rate with modulation (period: 100 seconds, amplitude: 1° C.). The experimental data were analyzed using commercially available software (TA Universal Analysis 2000/Trios software, TA Instruments). FIG. 6 shows a modulated differential scanning calorimetry measurement of 3-(6-Chloro-5-(2′-hydroxy-[1,1′-biphenyl]-4-yl)-1H-indazol-3-yl)propanoic acid.

### Embodiments

The following non-limiting embodiments provide illustrative examples of the invention, but do not limit the scope of the invention.

Embodiment 1. A composition comprising:
a) a compound of Formula I):

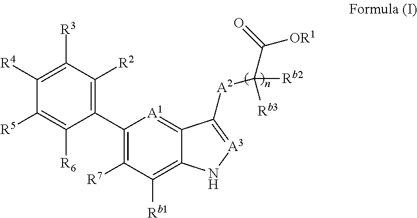

a pharmaceutically acceptable salt, a tautomer, or a pharmaceutically acceptable salt of the tautomer thereof, wherein:
   A¹ is CR⁸, or N;
   A² is CH₂, CHD, CD₂, S, O, or NH;
   A³ is CH, CD, or N;
   R¹ is H, D, C₁₋₈alkyl, C₃₋₆cycloalkyl, or 4-6 membered heterocycloalkyl, each of which is optionally substituted;
   R², R³, R⁵, and R⁶ are each independently H, D, OH, or halogen;
   R⁴ is monocyclic aryl, bicyclic aryl, monocyclic heteroaryl, or bicyclic heteroaryl, each of which is optionally substituted with R⁹, R¹⁰, R¹¹, R¹², or R¹³,
      wherein R⁹, R¹⁰, R¹¹, R¹², and R¹³ are each independently H, D, halogen, CN, oxo, C₁₋₈alkyl, C₃₋₆cycloalkyl, C₀₋₆alkylene-OR^{x}, C₁₋₆haloalkylene-OR^{x}, C₀₋₆alkylene(C₀₋₆haloalkyl)NR^{x}R^{y}, C₁₋₆alkylene(C₁₋₆haloalkyl)NRR^{y}, 4-6 membered heterocycloalkyl, C(O)OR^{x}, C₀₋₆alkylene-C(O)NRR^{y}, OC₁₋₃alkylene-heterocycloalkyl, OC₁₋₃alkylene-C(O)NR^{x}R^{y}, O(C₁₋₆alkyl)SO₂NR^{x}NR^{y}, NR^{x}R^{y}, NHSO₂R^{x}, SR^{x}, S—C₁₋₆alkylene-C(O)NR^{x}R^{y}, S(O)R^{x}R^{y}, SO₂R^{x}, SO₂NR^{x}R^{y}, S(O)(NR^{x})R^{y}, S(O)(NR^{x})R^{y}, or SO₂R^{x};
   wherein each R^{x} and R^{y} is independently H, D, C₁₋₆alkyl, C₁₋₆haloalkyl, C₁₋₆alkoxy, C₃₋₆cycloalkyl, C₁₋₆alkylene-amide, OC₀₋₂alkylene-heterocycloalkyl, 4-6 membered heterocycloalkyl, C(O)C₁₋₆alkyl, imino, or C₁₋₆alkylsulfonyl; or R^{x} and R^{y} together with the atoms to which R^{x} and R^{y} are bound can form an optionally substituted ring;
   R⁷ is C₁₋₃alkyl, C₃₋₆cycloalkyl, cyano, or halogen;
   R^{b1}, R^{b2}, and R^{b3} are each independently H or D;
   R³ is H, D, or halogen; and
   n is 0, 1, or 2;
b) a matrix component; and
c) a release modifier,
wherein the composition is an extended-release composition.

Embodiment 2. The composition of embodiment 1, consisting essentially of:
a) the compound of Formula (I), a pharmaceutically acceptable salt, a tautomer, or a pharmaceutically acceptable salt of the tautomer thereof;
b) the matrix component; and
c) the release modifier.

Embodiment 3. The composition of embodiment 1 or 2, wherein the matrix component is selected from the group consisting of: stearyl alcohol, polyglyceryl-4-stearate, and polyglyceryl-6-palmate.

Embodiment 4. The composition of any one of embodiments 1-3, wherein the matrix component is stearyl alcohol.

Embodiment 5. The composition of any one of embodiments 1-4, wherein the composition comprises the matrix component in an amount of from about 25% to about 90% w/w.

Embodiment 6. The composition of any one of embodiments 1-5, wherein the composition comprises the matrix component in an amount of from about 50% to about 90% w/w.

Embodiment 7. The composition of any one of embodiments 1-6, wherein the composition comprises the matrix component in an amount of about 68% w/w.

Embodiment 8. The composition of any one of embodiments 1-7, wherein the release modifier is selected from the group consisting of crospovidone, poloxamer 407, sodium starch glycolate Type A (SSG-A), polyglyceryl-4-stearate, polyglycerol-3-behenate.

Embodiment 9. The composition of any one of embodiments 1-8, wherein the release modifier is crospovidone.

Embodiment 10. The composition of any one of embodiments 1-9, wherein the composition comprises the release modifier in an amount of from about 3% to about 30% w/w.

Embodiment 11. The composition of any one of embodiments 1-10, wherein the composition comprises the release modifier in an amount of from about 5% to about 15% w/w.

Embodiment 12. The composition of any one of embodiments 1-11, wherein the composition comprises the release modifier in an amount of about 12% w/w.

Embodiment 13. The composition of any one of embodiments 1-12, wherein the composition comprises the compound of Formula (I), or a pharmaceutically acceptable salt, the tautomer, or the pharmaceutically acceptable salt of the tautomer thereof, in an amount of from about 5% to about 50% w/w.

Embodiment 14. The composition of any one of embodiments 1-13, wherein the composition comprises the compound of Formula (I), or a pharmaceutically acceptable salt, the tautomer, or the pharmaceutically acceptable salt of the tautomer thereof, in an amount of about 20% w/w.

Embodiment 15. The composition of any one of embodiments 1-14, wherein the composition comprises the compound of Formula (I), or the pharmaceutically acceptable salt, the tautomer, or the pharmaceutically acceptable salt of the tautomer thereof, wherein A¹ is CH or CF.

Embodiment 16. The composition of any one of embodiments 1-15, wherein the composition comprises the compound of Formula (I), or the pharmaceutically acceptable salt, the tautomer, or the pharmaceutically acceptable salt of the tautomer thereof, wherein A² is CH₂ or S.

Embodiment 17. The composition of any one of embodiments 1-16, wherein the composition comprises the compound of Formula (I), or the pharmaceutically acceptable salt, the tautomer, or the pharmaceutically acceptable salt of the tautomer thereof, wherein A³ is N.

Embodiment 18. The composition of any one of embodiments 1-17, wherein the composition comprises the compound of Formula (I), or the pharmaceutically acceptable salt, the tautomer, or the pharmaceutically acceptable salt of the tautomer thereof, wherein R¹ is H.

Embodiment 19. The composition of any one of embodiments 1-18, wherein the composition comprises the compound of Formula (I), or the pharmaceutically acceptable salt, the tautomer, or the pharmaceutically acceptable salt of the tautomer thereof, wherein R², R³, R⁵, and R⁶ are each independently H.

Embodiment 20. The composition of any one of embodiments 1-19, wherein the composition comprises the compound of Formula (I), or the pharmaceutically acceptable salt, the tautomer, or the pharmaceutically acceptable salt of the tautomer thereof, wherein R⁷ is Cl.

Embodiment 21. The composition of any one of embodiments 1-20, wherein the composition comprises the compound of Formula (I), or the pharmaceutically acceptable salt, the tautomer, or the pharmaceutically acceptable salt of the tautomer thereof, wherein R⁴ is phenyl, wherein R⁹, R¹⁰, R¹¹, R¹², and R¹³ are each independently H, D, Cl, F, CN, C₁₋₃alkyl, C₁₋₆alkylene-OH, C₁₋₆alkylene-OC₁₋₆alkyl, OH, OC₁₋₆alkyl, OC₁₋₆haloalkyl, O(C₁₋₃alkylene)heterocycloalkyl, O(C₁₋₃alkylene)-C(O)NR^{x}R^{y}, C₁₋₃alkylene-NR^{x}R^{y}, C(O)OH, C(O)OC₁₋₃ alkyl, C₀₋₂alkylene-C(O)NR^{x}R^{y}, SO₂NR^{x}R^{y}, S(O)(NR^{x})R^{y}, NR^{x}R^{y}, SR^{x}, or SO₂R^{x}.

Embodiment 22. The composition of any one of embodiments 1-21, wherein the composition comprises the compound of Formula (I), or the pharmaceutically acceptable salt, the tautomer, or the pharmaceutically acceptable salt of the tautomer thereof, wherein the compound has the structure Formula (II):

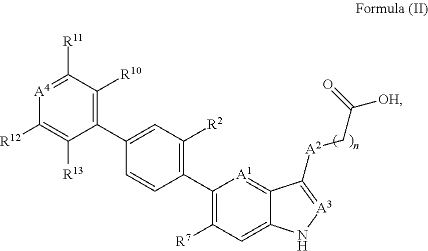

wherein:
   R² is H, D, or halogen;
   R⁷ is Cl or CN;
   A² is CH₂, CHD, CD₂, S, or NH;
   A⁴ is CR⁹ or N;
   R⁹ is H, D, F, Cl, C₁₋₃alkyl, C₁₋₃alkylene-O—C₁₋₃alkyl, C₁₋₃alkylene-NH₂, COOH, C(O)OC₁₋₃alkyl, C₁₋₃alkylene-C(O)NH₂, C(O)NHC₁₋₃alkyl, C(O)N(C₁₋₃alkyl)₂, C₁₋₆alkylene(C₁₋₆haloalkyl)NH₂, C₀₋₂alkylene-NH(C(O)C₁₋₃alkyl), OC₁₋₃alkyl, OC₁₋₃haloalkyl, OC₁₋₃alkylene-heterocycloalkyl, OC₁₋₃alkylene-C(O)NH₂, NHSO₂C₁₋₃alkyl, N(C₁₋₃ alkyl)(C(O)C₁₋₃alkyl), SC₁₋₃alkyl, S—C₁₋₃alkylene-C(O)NH₂, SO(NH)C₁₋₃alkyl, SO₂NH₂, or SO₂C₁₋₃alkyl;
   R¹⁰ is H, D, or OH;
   R¹¹ is H, D, halogen, CN, O(C₁₋₃alkyl), or O(C₁₋₃haloalkyl); or R⁹ and R¹¹ together with the carbon atom to which R⁹ and R¹¹ are bound form an optionally substituted ring;
   R¹² is H, D, OC₁₋₃alkyl, or C₁₋₃alkylene-OH;
   R¹³ is H, D, F, Cl, or C₁₋₃alkyl; and
   n is 1 or 2.

Embodiment 23. The composition of embodiment 22, wherein the composition comprises the compound of Formula (I), or the pharmaceutically acceptable salt, the tautomer, or the pharmaceutically acceptable salt of the tautomer thereof, wherein R¹⁰ is OH.

Embodiment 24. The composition of embodiment 22 or 23, wherein the composition comprises the compound of Formula (I), or the pharmaceutically acceptable salt, the tautomer, or the pharmaceutically acceptable salt of the tautomer thereof, wherein R⁷ is Cl.

Embodiment 25. The composition of any one of embodiments 22-24, wherein the composition comprises the compound of Formula (I), or the pharmaceutically acceptable salt, the tautomer, or the pharmaceutically acceptable salt of the tautomer thereof, wherein A² is CH₂ or S.

Embodiment 26. The composition of embodiment 22, wherein the composition comprises the compound of Formula (I), or the pharmaceutically acceptable salt, the tautomer, or the pharmaceutically acceptable salt of the tautomer thereof, wherein:
R⁹ is H or —C(O)NR^{x}R^{y}, wherein each R^{x} and R^{y} are independently H or C₁₋₆alkyl; and
R¹¹ is H or —O(C₁₋₃alkyl); or R⁹ and R¹¹ together with the carbon atom to which R⁹ and R¹ are bound form an optionally substituted ring.

Embodiment 27. The composition of any one of embodiments 1-14, wherein the compound of Formula (I) is selected from the group consisting of:
3-(6-Chloro-5-(2′-hydroxy-[1,1′-biphenyl]-4-yl)-1H-indazol-3-yl)propanoic acid;
3-(6-chloro-5-(2′-hydroxy-6′-methyl-[1,1′-biphenyl]-4-yl)-1H-indazol-3-yl)propanoic acid;
3-(6-chloro-5-(2′-hydroxy-3′-methoxy-[1,1′-biphenyl]-4-yl)-1H-indazol-3-yl)propanoic acid;
3-(6-chloro-5-(2′-hydroxy-3′-methoxy-6′-methyl-[1,1-biphenyl]-4-yl)-1H-indazol-3-yl)propanoic acid;
3-(6-chloro-5-(4-(7-hydroxy-2,3-dihydrobenzofuran-6-yl)phenyl)-1H-indazol-3-yl)propanoic acid;
3-(6-chloro-5-(2′-hydroxy-4′-(methoxymethyl)-[1,1′-biphenyl]-4-yl)-1H-indazol-3-yl)propanoic acid;
3-(6-chloro-5-(2′-hydroxy-4′,6′-dimethyl-[1,1′-biphenyl]-4-yl)-1H-indazol-3-yl)propanoic acid;
3-(6-chloro-5-(3′-fluoro-2′-hydroxy-6′-methyl-[1,1′-biphenyl]-4-yl)-1H-indazol-3-yl)propanoic acid;
3-(6-chloro-5-(4′-fluoro-2′-hydroxy-3′-methoxy-[1,1′-biphenyl]-4-yl)-1H-indazol-3-yl)propanoic acid;
3-(6-chloro-5-(4′-(dimethylcarbamoyl)-[1,1′-biphenyl]-4-yl)-1H-indazol-3-yl)propanoic acid;
4-(6-chloro-5-(2′-hydroxy-3′-methoxy-[1,1′-biphenyl]-4-yl)-1H-indazol-3-yl)butanoic acid;
3-(6-chloro-5-(4′-(methylcarbamoyl)-[1,1′-biphenyl]-4-yl)-1H-indazol-3-yl)propanoic acid;
6-((3-(6-chloro-5-(2′-hydroxy-[1,1′-biphenyl]-4-yl)-1H-indazol-3-yl)propanoyl)oxy)-3,4,5-trihydroxytetrahydro-2H-pyran-2-carboxylic acid; and
6-((4′-(3-(2-carboxyethyl)-6-chloro-1H-indazol-5-yl)-[1,1′-biphenyl]-2-yl)oxy)-3,4,5-trihydroxytetrahydro-2H-pyran-2-carboxylic acid,
   or a pharmaceutically acceptable salt, a tautomer, or a pharmaceutically acceptable salt of the tautomer thereof.

Embodiment 28. The composition of any one of embodiments 1-14, wherein the compound of Formula (I) is 3-(6-chloro-5-(2′-hydroxy-3′-methoxy-[1,1′-biphenyl]-4-yl)-1H-indazol-3-yl)propanoic acid, a pharmaceutically acceptable salt, a tautomer, or a pharmaceutically acceptable salt of the tautomer thereof.

Embodiment 29. The composition of any one of embodiments 1-14, wherein the compound of Formula (I) is 3-(6-Chloro-5-(2′-hydroxy-[1,1′-biphenyl]-4-yl)-1H-indazol-3-yl)propanoic acid, a pharmaceutically acceptable salt, a tautomer, or a pharmaceutically acceptable salt of the tautomer thereof.

Embodiment 30. The composition of any one of embodiments 1-14, wherein the compound of Formula (I) is 4-(6-chloro-5-(2′-hydroxy-3′-methoxy-[1,1′-biphenyl]-4-yl)-1H-indazol-3-yl)butanoic acid, a pharmaceutically acceptable salt, a tautomer, or a pharmaceutically acceptable salt of the tautomer thereof.

Embodiment 31. The composition of any one of embodiments 1-14, wherein the compound is 3-(6-chloro-5-(2′-hydroxy-4′-(methoxymethyl)-[1,1′-biphenyl]-4-yl)-1H-indazol-3-yl)propanoic acid, a pharmaceutically acceptable salt, a tautomer, or a pharmaceutically acceptable salt of the tautomer thereof.

Embodiment 32. The composition of any one of embodiments 1-14, wherein the compound is 6-((3-(6-chloro-5-(2′-hydroxy-[1,1′-biphenyl]-4-yl)-1H-indazol-3-yl)propanoyl)oxy)-3,4,5-trihydroxytetrahydro-2H-pyran-2-carboxylic acid, a pharmaceutically acceptable salt, a tautomer, or a pharmaceutically acceptable salt of the tautomer thereof.

Embodiment 33. The composition of any one of embodiments 1-32, wherein the composition comprises the compound of Formula (I), a pharmaceutically acceptable salt, a tautomer, or a pharmaceutically acceptable salt of the tautomer thereof, in an amount of from about 1 mg to about 500 mg.

Embodiment 34. The composition of any one of embodiments 1-33, wherein the composition comprises the compound of Formula (I), a pharmaceutically acceptable salt, a tautomer, or a pharmaceutically acceptable salt of the tautomer thereof, in an amount of about 10 mg.

Embodiment 35. The composition of any one of embodiments 1-34, wherein the composition is formulated for extended release.

Embodiment 36. The composition of any one of embodiments 1-35, wherein the composition is formulated for oral administration.

Embodiment 37. The composition of any one of embodiments 1-36, wherein the composition comprises the compound of Formula (I), a pharmaceutically acceptable salt, a tautomer, or a pharmaceutically acceptable salt of the tautomer thereof, in the form of a microsphere particle.

Embodiment 38. The composition of embodiment 37, wherein an amount of the microsphere particle has an average particle size (D50) of from about 80 μm to about 400 μm.

Embodiment 39. The composition of embodiment 37 or 38, wherein an amount of the microsphere particle has an average particle size (D50) of from about 175 μm to about 225 μm.

Embodiment 40. The composition of any one of embodiments 37-39, wherein the microsphere particle is coated with a coating to form a microcapsule.

Embodiment 41. The composition of embodiment 40, wherein the coating comprises methacrylic acid, ethyl acrylate, or methyl methacrylate.

Embodiment 42. The composition of embodiment 40 or 41, wherein the coating comprises a methacrylic acid-ethyl acrylate copolymer.

Embodiment 43. The composition of embodiment 40 or 41, wherein the coating comprises a methacrylic acid-methyl methacrylate copolymer.

Embodiment 44. The composition of embodiment 40, wherein the coating comprises a poly(methyl acrylate-co-methyl methacrylate-co-methacrylic acid).

Embodiment 45. The composition of any one of embodiments 40-44, wherein an amount of the microcapsule has an average particle size (D50) of from about 200 μm to about 300 μm.

Embodiment 46. The composition of any one of embodiments 1-45, wherein the composition has a time to 80% release in fasted state simulated intestinal fluid (FaSSIF) of from about 4 hours to about 50 hours.

Embodiment 47. The composition of any one of embodiments 1-46, wherein the composition has a time to 80% release in FaSSIF of from about 8 hours to about 11 hours.

Embodiment 48. The composition of any one of embodiments 1-46, wherein the composition has a time to 80% release in FaSSIF of from about 15 hours to about 20 hours.

Embodiment 49. A method for treating a condition, comprising administering to a subject in need thereof a therapeutically effective amount of the composition of any of embodiments 1-48, wherein the condition is an inflammatory condition, an autoimmune condition, or a functional gastrointestinal disorder.

Embodiment 50. The method of embodiment 49, wherein the inflammatory condition or the autoimmune condition is selected from the group consisting of inflammatory bowel disease, ulcerative colitis, Crohn's disease, celiac disease, atopic dermatitis, psoriasis, rheumatoid arthritis, and lupus.

Embodiment 51. The method of embodiment 49, wherein the functional gastrointestinal disorder is selected from the group consisting of irritable bowel syndrome, functional diarrhea, celiac disease, and functional constipation.

Embodiment 52. The method of any one of embodiments 49-51, wherein the administering is oral.

Embodiment 53. The method of any one of embodiments 49-52, wherein the administering is once daily.

Embodiment 54. The method of any one of embodiments 49-53, wherein the therapeutically effective amount is from about 1 mg to about 2500 mg of the compound, a pharmaceutically acceptable salt, the tautomer, or the pharmaceutically acceptable salt of the tautomer thereof.

Embodiment 55. The method of any one of embodiments 49-53, wherein the therapeutically effective amount is from about 10 mg of the compound, a pharmaceutically acceptable salt, the tautomer, or the pharmaceutically acceptable salt of the tautomer thereof.

Embodiment 56. Use of the composition of any one of embodiments 1-48, in the manufacture of a medicament for the treatment of an inflammatory condition, an autoimmune condition, or a functional gastrointestinal disorder.

Embodiment 57. A method of making a composition of embodiment 1, comprising:
a) admixing, blending, and milling the compound of Formula (I), a pharmaceutically acceptable salt, a tautomer, or a pharmaceutically acceptable salt of the tautomer thereof; the matrix component; and the release modifier to form a mixture;
b) subjecting the mixture to a melt-spray-congeal process to form microsphere particles.

Embodiment 58. The method of embodiment 57, further comprising coating the microsphere particles to form a microcapsule.

Embodiment 59. A compound of Formula (I):

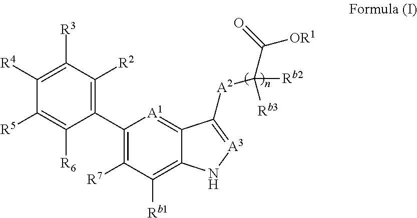

or a pharmaceutically acceptable salt, a tautomer, or a pharmaceutically acceptable salt of the tautomer thereof, wherein:
   A¹ is CR⁸, or N;
   A² is CH₂, CHD, CD₂, S, O, or NH;
   A³ is CH, CD, or N;
   R¹ is H, D, C₁₋₈alkyl, C₃₋₆cycloalkyl, or 4-6 membered heterocycloalkyl, each of which is optionally substituted;
   R², R³, R⁵, and R⁶ are each independently H, D, OH, or halogen;
   R⁴ is monocyclic aryl, bicyclic aryl, monocyclic heteroaryl, or bicyclic heteroaryl, each of which is optionally substituted with R⁹, R¹⁰, R¹¹, R¹², or R¹³,
      wherein R⁹, R¹⁰, R¹¹, R¹², and R¹³ are each independently H, D, halogen, CN, oxo, C₁₋₈alkyl, C₃₋₆cycloalkyl, C₀₋₆alkylene-OR^{x}, C₁₋₆haloalkylene-OR^{x}, C₀₋₆alkylene(C₀₋₆haloalkyl)NR^{x}R^{y}, C₁₋₆alkylene(C₁₋₆haloalkyl)NRR^{y}, 4-6 membered heterocycloalkyl, C(O)OR^{x}, C₀₋₆alkylene-C(O)NRR^{y}, OC₁₋₃alkylene-heterocycloalkyl, OC₁₋₃alkylene-C(O)NR^{x}R^{y}, O(C₁₋₆alkyl)SO₂NR^{x}NR^{y}, NR^{x}R^{y}, NHSO₂R^{x}, SR^{x}, S—C₁₋₆alkylene-C(O)NR^{x}R^{y}, S(O)R^{x}R^{y}, SO₂R^{x}, SO₂NR^{x}R^{y}, S(O)(NR^{x})R^{y}, S(O)(NR^{x})R^{y}, or SO₂R^{x};
   wherein each R^{x} and R^{y} is independently H, D, C₁₋₆alkyl, C₁₋₆haloalkyl, C₁₋₆alkoxy, C₃₋₆cycloalkyl, C₁₋₆alkylene-amide, OC₀₋₂alkylene-heterocycloalkyl, 4-6 membered heterocycloalkyl, C(O)C₁₋₆alkyl, imino, or C₁₋₆alkylsulfonyl; or R^{x} and R^{y} together with the atoms to which R^{x} and R^{y} are bound can form an optionally substituted ring;
   R⁷ is C₁₋₃alkyl, C₃₋₆cycloalkyl, cyano, or halogen;
   R^{b1}, R^{b2}, and R^{b3} are each independently H or D;
   R³ is H, D, or halogen; and
   n is 0, 1, or 2,
wherein the compound is amorphous.

Each of the embodiments described herein may be combined with any other embodiment(s) described herein not inconsistent with the embodiment(s) with which it is combined. In addition, any of the compounds described in the Examples, or pharmaceutically acceptable salts thereof, may be claimed individually or grouped together with one or more other compounds of the Examples, or pharmaceutically acceptable salts thereof, for any of the embodiment(s) described herein. Furthermore, each of the embodiments described herein envisions within its scope pharmaceutically acceptable salts of the compounds described herein.

It will be apparent to those skilled in the art that various modifications and variations may be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

All references cited herein, including patents, patent applications, papers, textbooks, and the like, and the references cited therein, to the extent that they are not already, are hereby incorporated by reference in their entireties. In the event that one or more of the incorporated literature and similar materials differs from or contradicts this application, including but not limited to defined terms, term usage, described techniques, or the like, this application controls.

What is claimed is:

1. A composition comprising:
a) a compound of Formula (I): 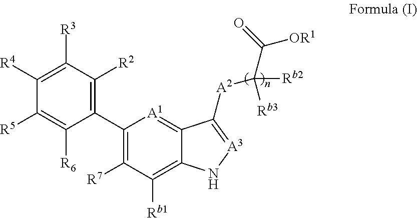
a pharmaceutically acceptable salt, a tautomer, or a pharmaceutically acceptable salt of the tautomer thereof, wherein:
A¹ is CR⁸, or N;
A² is CH₂, CHD, CD₂, S, O, or NH;
A³ is CH, CD, or N;
R¹ is H, D, C₁₋₈alkyl, C₃₋₆cycloalkyl, or 4-6 membered heterocycloalkyl, each of which is optionally substituted;
R², R³, R⁵, and R⁶ are each independently H, D, OH, or halogen;
R⁴ is monocyclic aryl, bicyclic aryl, monocyclic heteroaryl, or bicyclic heteroaryl, each of which is optionally substituted with R⁹, R¹⁰, R¹¹, R¹², or R¹³,
wherein R⁹, R¹⁰, R¹¹, R¹², and R¹³ are each independently H, D, halogen, CN, oxo, C₁₋₈alkyl, C₃₋₆cycloalkyl, examC₀₋₆alkylene-OR^{x}, C₁₋₆haloalkylene-OR^{x}, C₀₋₆alkylene(C₀₋₆haloalkyl)NRR^{y}, C₁₋₆alkylene(C₁₋₆haloalkyl)NRR^{y}, 4-6 membered heterocycloalkyl, C(O)OR^{x}, C₀₋₆alkylene-C(O)NR^{x}R^{y}, OC₁₋₃alkylene-heterocycloalkyl, OC₁₋₃alkylene-C(O)NR^{x}R^{y}, O(C₁₋₆alkyl)SO₂NR^{x}NR^{y}, NR^{x}R^{y}, NHSO₂R^{x}, SR^{x}, S—C₁₋₆alkylene-C(O)NR^{x}R^{y}, S(O)R^{x}R^{y}, SO₂R^{x}, SO₂NR^{x}R^{y}, S(O)(NR^{x})R^{y}, S(O)(NR^{x})R^{y}, or SO₂R^{x}; wherein each R^{x} and R^{y} is independently H, D, C₁₋₆alkyl, C₁₋₆haloalkyl, C₁₋₆alkoxy, C₃₋₆cycloalkyl, C₁₋₆alkylene-amide, OC₀₋₂alkylene-heterocycloalkyl, 4-6 membered heterocycloalkyl, C(O)C₁₋₆alkyl, imino, or C₁₋₆alkylsulfonyl; or R^{x} and R^{y} together with the atoms to which R^{x} and R^{y} are bound can form an optionally substituted ring;
R⁷ is C₁₋₃alkyl, C₃₋₆cycloalkyl, cyano, or halogen;
R^{b1}, R^{b2}, and R^{b3} are each independently H or D;
R³ is H, D, or halogen; and
n is 0, 1, or 2;
b) a matrix component; and
c) a release modifier,
wherein the composition is an extended-release composition.

2. The composition of claim 1, consisting essentially of:
a) the compound of Formula (I), a pharmaceutically acceptable salt, a tautomer, or a pharmaceutically acceptable salt of the tautomer thereof;
b) the matrix component; and
c) the release modifier.

3. The composition of claim 1, wherein the matrix component is selected from the group consisting of: stearyl alcohol, polyglyceryl-4-stearate, and polyglyceryl-6-palmate.

4. The composition of claim 1, wherein the composition comprises the matrix component in an amount of from about 25% to about 90% w/w.

5. The composition of claim 1, wherein the release modifier is selected from the group consisting of crospovidone, poloxamer 407, sodium starch glycolate Type A (SSG-A), polyglyceryl-4-stearate, polyglycerol-3-behenate.

6. The composition of claim 1, wherein the composition comprises the release modifier in an amount of from about 3% to about 30% w/w.

7. The composition of claim 1, wherein the composition comprises the compound of Formula (I), or a pharmaceutically acceptable salt, the tautomer, or the pharmaceutically acceptable salt of the tautomer thereof, in an amount of from about 5% to about 50% w/w.

8. The composition of claim 1, wherein the composition comprises the compound of Formula (I), or the pharmaceutically acceptable salt, the tautomer, or the pharmaceutically acceptable salt of the tautomer thereof, wherein A¹ is CH or CF; A² is CH₂ or S; and A³ is N.

9. The composition of claim 1, wherein the composition comprises the compound of Formula (I), or the pharmaceutically acceptable salt, the tautomer, or the pharmaceutically acceptable salt of the tautomer thereof, wherein R¹, R², R³, R⁵, and R⁶ are each independently H; and R⁷ is Cl.

10. The composition of claim 1, wherein the composition comprises the compound of Formula (I), or the pharmaceutically acceptable salt, the tautomer, or the pharmaceutically acceptable salt of the tautomer thereof, wherein R⁴ is phenyl, wherein R⁹, R¹⁰, R¹¹, R¹², and R¹³ are each independently H, D, Cl, F, CN, C₁₋₃alkyl, C₁₋₆alkylene-OH, C₁₋₆alkylene-OC₁₋₆alkyl, OH, OC₁₋₆alkyl, OC₁₋₆haloalkyl, O(C₁₋₃alkylene)heterocycloalkyl, O(C₁₋₃alkylene)-C(O)NR^{x}R^{y}, C₁₋₃alkylene-NR^{x}R^{y}, C(O)OH, C(O)OC₁₋₃alkyl, C₀₋₂alkylene-C(O)NR^{x}R^{y}, SO₂NR^{x}R^{y}, S(O)(NR^{x})R^{y}, NR^{x}R^{y}, SR^{x}, or SO₂R^{x}.

11. The composition of claim 1, wherein the compound of Formula (I) is selected from the group consisting of:
3-(6-Chloro-5-(2′-hydroxy-[1,1′-biphenyl]-4-yl)-1H-indazol-3-yl)propanoic acid;
3-(6-chloro-5-(2′-hydroxy-6′-methyl-[1,1′-biphenyl]-4-yl)-1H-indazol-3-yl)propanoic acid;
3-(6-chloro-5-(2′-hydroxy-3′-methoxy-[1,1′-biphenyl]-4-yl)-1H-indazol-3-yl)propanoic acid;
3-(6-chloro-5-(2′-hydroxy-3′-methoxy-6′-methyl-[1,1′-biphenyl]-4-yl)-1H-indazol-3-yl)propanoic acid;
3-(6-chloro-5-(4-(7-hydroxy-2,3-dihydrobenzofuran-6-yl)phenyl)-1H-indazol-3-yl)propanoic acid;
3-(6-chloro-5-(2′-hydroxy-4′-(methoxymethyl)-[1,1-biphenyl]-4-yl)-1H-indazol-3-yl)propanoic acid;
3-(6-chloro-5-(2′-hydroxy-4′,6′-dimethyl-[1,1-biphenyl]-4-yl)-1H-indazol-3-yl)propanoic acid;
3-(6-chloro-5-(3′-fluoro-2′-hydroxy-6′-methyl-[1,1′-biphenyl]-4-yl)-1H-indazol-3-yl)propanoic acid;
3-(6-chloro-5-(4′-fluoro-2′-hydroxy-3′-methoxy-[1,1′-biphenyl]-4-yl)-1H-indazol-3-yl)propanoic acid;
3-(6-chloro-5-(4′-(dimethylcarbamoyl)-[1,1′-biphenyl]-4-yl)-1H-indazol-3-yl)propanoic acid;
4-(6-chloro-5-(2′-hydroxy-3′-methoxy-[1,1′-biphenyl]-4-yl)-1H-indazol-3-yl)butanoic acid;
3-(6-chloro-5-(4′-(methylcarbamoyl)-[1,1′-biphenyl]-4-yl)-1H-indazol-3-yl)propanoic acid;
6-((3-(6-chloro-5-(2′-hydroxy-[1,1′-biphenyl]-4-yl)-1H-indazol-3-yl)propanoyl)oxy)-3,4,5-trihydroxytetrahydro-2H-pyran-2-carboxylic acid; and
6-((4′-(3-(2-carboxyethyl)-6-chloro-1H-indazol-5-yl)-[1,1′-biphenyl]-2-yl)oxy)-3,4,5-trihydroxytetrahydro-2H-pyran-2-carboxylic acid,
or a pharmaceutically acceptable salt, a tautomer, or a pharmaceutically acceptable salt of the tautomer thereof.

12. The composition of claim 1, wherein the composition comprises the compound of Formula (I), a pharmaceutically acceptable salt, a tautomer, or a pharmaceutically acceptable salt of the tautomer thereof, in an amount of from about 1 mg to about 500 mg.

13. The composition of claim 1, wherein the composition is formulated for oral administration.

14. The composition of claim 1, wherein the composition comprises the compound of Formula (I), a pharmaceutically acceptable salt, a tautomer, or a pharmaceutically acceptable salt of the tautomer thereof, in the form of a microsphere particle.

15. The composition of claim 14, wherein an amount of the microsphere particle has an average particle size (D50) of from about 80 μm to about 400 μm.

16. The composition of claim 14, wherein the microsphere particle is coated with a coating to form a microcapsule.

17. The composition of claim 16, wherein the coating comprises methacrylic acid, ethyl acrylate, or methyl methacrylate.

18. The composition of claim 16, wherein an amount of the microcapsule has an average particle size (D50) of from about 200 μm to about 300 μm.

19. A method for treating a condition, comprising administering to a subject in need thereof a therapeutically effective amount of the composition of claim 1, wherein the condition is an inflammatory condition, an autoimmune condition, or a functional gastrointestinal disorder.

20. A method of making a composition of claim 1, comprising:
a) admixing, blending, and milling the compound of Formula (I), a pharmaceutically acceptable salt, a tautomer, or a pharmaceutically acceptable salt of the tautomer thereof; the matrix component; and the release modifier to form a mixture;
b) subjecting the mixture to a melt-spray-congeal process to form microsphere particles.
